SEQUENCE LISTING (46 sequences)

SEQ ID NO: 1 - DNA, 1172 bp, Homo sapiens
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctg

SEQ ID NO: 2 - DNA, 1329 bp, Homo sapiens
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 3 - DNA, 1245 bp, Homo sapiens
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 4 - DNA, 1239 bp, Homo sapiens
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctga

SEQ ID NO: 5 - DNA, 930 bp, Homo sapiens
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtga

SEQ ID NO: 6 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 7 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 8 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 9 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 10 - protein, 309 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 11 - DNA, 1173 bp, Mus musculus
atgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 12 - DNA, 1245 bp, Mus musculus
atgcactactgtgtgctgagcacctttttgctcctgcatctggtcccggtggcgctcagtctgtctacctgcagcaccctcgacatggatcagtttatgcgcaagaggatcgaggccatccgcgggcagatcctgagcaagctgaagctcaccagccccccggaagactatccggagccggatgaggtccccccggaggtgatttccatctacaacagtaccagggacttactgcaggagaaggcaagccggagggcagccgcctgcgagcgcgagcggagcgacgaggagtactacgccaaggaggtttataaaatcgacatgccgtcccacctcccctccgaaaatgccatcccgcccactttctacagaccctacttcagaatcgtccgctttgatgtctcaacaatggagaaaaatgcttcgaatctggtgaaggcagagttcagggtcttccgcttgcaaaaccccaaagccagagtggccgagcagcggattgaactgtatcagatccttaaatccaaagacttaacatctcccacccagcgctacatcgatagcaaggttgtgaaaaccagagcggagggtgaatggctctccttcgacgtgacagacgctgtgcaggagtggcttcaccacaaagacaggaacctggggtttaaaataagtttacactgcccctgctgtaccttcgtgccgtctaataattacatcatcccgaataaaagcgaagagctcgaggcgagatttgcaggtattgatggcacctctacatatgccagtggtgatcagaaaactataaagtccactaggaaaaaaaccagtgggaagaccccacatctcctgctaatgttgttgccctcctacagactggagtcacaacagtccagccggcggaagaagcgcgctttggatgctgcctactgctttagaaatgtgcaggataattgctgccttcgccctctttacattgattttaagagggatcttggatggaaatggatccatgaacccaaagggtacaatgctaacttctgtgctggggcatgcccatatctatggagttcagacactcaacacaccaaagtcctcagcctgtacaacaccataaatcccgaagcttccgcttccccttgctgtgtgtcccaggatctggaaccactgaccattctctattacattggaaatacgcccaagatcgaacagctttccaatatgattgtcaagtcttgtaaatgcagctaa

SEQ ID NO: 13 - DNA, 1239 bp, Mus musculus
atgaagatgcacttgcaaagggctctggtagtcctggccctgctgaacttggccacaatcagcctctctctgtccacttgcaccacgttggacttcggccacatcaagaagaagagggtggaagccattaggggacagatcttgagcaagctcaggctcaccagcccccctgagccatcggtgatgacccacgtcccctatcaggtcctggcactttacaacagcacccgggagttgctggaagagatgcacggggagagggaggaaggctgcactcaggagacctcggagtctgagtactatgccaaagagatccataaattcgacatgatccagggactggcggagcacaatgaactggccgtctgccccaaaggaattacctctaaggtttttcgtttcaatgtgtcctcagtggagaaaaatggaaccaatctgttccgggcagagttccgggtcttgcgggtgcccaaccccagctccaagcgcacagagcagagaattgagctcttccagatacttcgaccggatgagcacatagccaagcagcgctacataggtggcaagaatctgcccacaaggggcaccgctgaatggctgtctttcgatgtcactgacactgtgcgcgagtggctgttgaggagagagtccaacttgggtctggaaatcagcatccactgtccatgtcacacctttcagcccaatggagacatactggaaaatgttcatgaggtgatggaaatcaaattcaaaggagtggacaatgaagatgaccatggccgtggagacctggggcgtctcaagaagcaaaaggatcaccacaacccacacctgatcctcatgatgatccccccacaccgactggacagcccaggccagggcagtcagaggaagaagagggccctggacaccaattactgcttccgcaacctggaggagaactgctgtgtacgccccctttatattgacttccggcaggatctaggctggaaatgggtccacgaacctaagggttactatgccaacttctgctcaggcccttgcccatacctccgcagcgcagacacaacccatagcacggtgcttggactatacaacaccctgaacccagaggcgtctgcctcgccatgctgcgtcccccaggacctggagcccctgaccatcttgtactatgtgggcagaacccccaaggtggagcagctgtccaacatggtggtgaagtcgtgtaagtgcagctga

SEQ ID NO: 14 - DNA, 130 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 130 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 2056 bp, Homo sapiens
cctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccata

SEQ ID NO: 17 - DNA, 542 bp, Artificial Sequence
  Description of Artificial Sequence: Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 18 - DNA, 215 bp, Artificial Sequence
  Description of Artificial Sequence: Synthetic polynucleotide
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 19 - DNA, 22 bp, Artificial Sequence
  Description of Artificial Sequence: Synthetic primer
aaaggcaatccaggcaatatca

SEQ ID NO: 20 - DNA, 21 bp, Artificial Sequence
  Description of Artificial Sequence: Synthetic primer
tggttctggtatggactctcc

SEQ ID NO: 21 - DNA, 21 bp, Artificial Sequence
  Description of Artificial Sequence: Synthetic primer
aggtcggtgtgaacggatttg

SEQ ID NO: 22 - DNA, 23 bp, Artificial Sequence
  Description of Artificial Sequence: Synthetic primer
tgtagaccatgtagttgaggtca

SEQ ID NO: 23 - DNA, 23 bp, Artificial Sequence
  Description of Artificial Sequence: Synthetic primer
tgacctcaactacatggtctaca

SEQ ID NO: 24 - DNA, 19 bp, Artificial Sequence
  Description of Artificial Sequence: Synthetic primer
cttcccattctcggccttg

SEQ ID NO: 25 - DNA, 19 bp, Artificial Sequence
  Description of Artificial Sequence: Synthetic primer
tgctggcttccgagtcttc

SEQ ID NO: 26 - DNA, 19 bp, Artificial Sequence
  Description of Artificial Sequence: Synthetic primer
cggggtcgttctcgaacac

SEQ ID NO: 27 - DNA, 22 bp, Artificial Sequence
  Description of Artificial Sequence: Synthetic primer
cgaagactacagttctgccatt

SEQ ID NO: 28 - DNA, 22 bp, Artificial Sequence
  Description of Artificial Sequence: Synthetic primer
gacgtttcagaggttctcagag

SEQ ID NO: 29 - DNA, 22 bp, Artificial Sequence
  Description of Artificial Sequence: Synthetic primer
gcaactgttcctgaactcaact

SEQ ID NO: 30 - DNA, 21 bp, Artificial Sequence
  Description of Artificial Sequence: Synthetic primer
atcttttggggtccgtcaact

SEQ ID NO: 31 - DNA, 23 bp, Artificial Sequence
  Description of Artificial Sequence: Synthetic primer
tagtccttcctaccccaatttcc

SEQ ID NO: 32 - DNA, 21 bp, Artificial Sequence
  Description of Artificial Sequence: Synthetic primer
ttggtccttagccactccttc

SEQ ID NO: 33 - DNA, 22 bp, Artificial Sequence
  Description of Artificial Sequence: Synthetic primer
agcaagaaactcttccaagcaa

SEQ ID NO: 34 - DNA, 23 bp, Artificial Sequence
  Description of Artificial Sequence: Synthetic primer
gtgagattcgtcagattcatccg

SEQ ID NO: 35 - DNA, 19 bp, Artificial Sequence
  Description of Artificial Sequence: Synthetic primer
ctcccgtggcttctagtgc

SEQ ID NO: 36 - DNA, 22 bp, Artificial Sequence
  Description of Artificial Sequence: Synthetic primer
gccttagtttggacaggatctg

SEQ ID NO: 37 - DNA, 20 bp, Artificial Sequence
  Description of Artificial Sequence: Synthetic primer
gagcccgaagcggactacta

SEQ ID NO: 38 - DNA, 23 bp, Artificial Sequence
  Description of Artificial Sequence: Synthetic primer
tggttttctcatagatggcgttg

SEQ ID NO: 39 - DNA, 19 bp, Artificial Sequence
  Description of Artificial Sequence: Synthetic primer
cttcgacgtgacagacgct

SEQ ID NO: 40 - DNA, 22 bp, Artificial Sequence
  Description of Artificial Sequence: Synthetic primer
gcaggggcagtgtaaacttatt

SEQ ID NO: 41 - DNA, 19 bp, Artificial Sequence
  Description of Artificial Sequence: Synthetic primer
cctggccctgctgaacttg

SEQ ID NO: 42 - DNA, 21 bp, Artificial Sequence
  Description of Artificial Sequence: Synthetic primer
ttgatgtggccgaagtccaac

SEQ ID NO: 43 - DNA, 20 bp, Artificial Sequence
  Description of Artificial Sequence: Synthetic primer
cctactctgtgtcactcccg

SEQ ID NO: 44 - DNA, 21 bp, Artificial Sequence
  Description of Artificial Sequence: Synthetic primer
cacgtactgccggaagaaatc

SEQ ID NO: 45 - DNA, 23 bp, Artificial Sequence
  Description of Artificial Sequence: Synthetic primer
ccctcacactcagatcatcttct

SEQ ID NO: 46 - DNA, 19 bp, Artificial Sequence
  Description of Artificial Sequence: Synthetic primer
gctacgacgtgggctacag

(19) United States
(12) Patent Application Publication
(10) Pub. No.: US 2023/0277685 A1
(43) Pub. Date: Sep. 7, 2023

(54) TGFß THERAPY FOR OCULAR AND NEURODEGENERATIVE DISEASES

(71) Applicant: PRESIDENT AND FELLOWS OF HARVARD COLLEGE, Cambridge, MA (US)

(72) Inventors: Sean K. WANG, Cambridge, MA (US); Constance L. CEPKO, Cambridge, MA (US)

(73) Assignee: PRESIDENT AND FELLOWS OF HARVARD COLLEGE, Cambridge, MA (US)

(21) Appl. No.: 17/917,109

(22) PCT Filed: Apr. 8, 2021
(86) PCT No.: PCT/US2021/026389

§ 371 Date: Oct. 5, 2022

Related U.S. Application Data
(60) Provisional application No. 63/006,817, filed on Apr. 8, 2020.

(51) Int. Cl. A61K 48/00; C12N 15/86; A61P 27/02

(52) U.S. Cl. CPC A61K 48/0058; A61P 27/02; C12N 15/86; C12N 2750/14143; C12N 2830/008; C12N 2830/48

(57) ABSTRACT

Provided herein are methods and compositions related to the treatment of a neurodegenerative disease or an ocular disease.

### CROSS-REFERENCE TO RELATED APPLICATIONS

This application is a 35 U.S.C. §371 National Phase Entry Application of International Application No. PCT/US2021/026389 filed Apr. 8, 2021, which designates the U.S. and claims benefit under 35 U.S.C. § 119(e) of U.S. Provisional Application No. 63/006,817 filed Apr. 8, 2020, the contents of which are incorporated herein by reference in its entirety.

### SEQUENCE LISTING

[0001.1] The instant application contains a Sequence Listing which has been submitted electronically in ASCII format and is hereby incorporated by reference in its entirety. Said ASCII copy, created on Mar. 10, 2023, is named 002806-097280USPX_SL.txt and is 42,016 bytes in size.

### TECHNICAL FIELD

The technology described herein relates to methods and compositions for the treatment of ocular and neurodegenerative diseases.

### BACKGROUND

Neurodegenerative and other ocular diseases can lead to significant loss of daylight, color, and high-acuity vision in affected individuals. Gene complementation with adeno-associated viral (AAV) vectors is one strategy to treat degenerative ocular disease. However, the tremendous number of loci with causal mutations in certain neurodegenerative and ocular diseases makes the implementation of gene therapy challenging. Thus, there is an unmet need for gene therapy treatments for neurodegenerative and ocular diseases that are more broadly beneficial for patients with cone degeneration and other forms of neurodegeneration.

### SUMMARY

The compositions and methods described herein, are based, in part, on the discovery that Transforming Growth Factor β (TGF-β) delivered to the retina protects retinal cone photoreceptor cells from degeneration in several different animal models of the neurodegenerative ocular disease retinitis pigmentosa, preserving ocular function. The data demonstrate that the protective function of TGF-β requires the presence of retinal microglia, the resident macrophages of the CNS, and therefore points to microglia as therapeutic targets not only in retinal or ocular disorders, but also in other neurodegenerative conditions in neuronal tissues associated with microglia. In various embodiments, TGF-β polypeptides delivered to neuronal tissues comprising microglia, e.g., via viral vector, can reduce neuronal cell degeneration in ocular and other neurodegenerative diseases.

In one aspect, described herein is an engineered vector comprising a retina-specific promoter operably linked to a nucleic acid sequence encoding a transforming growth factor beta (TGF-β) polypeptide.

In another aspect, described herein is a pharmaceutical composition for the treatment of an ocular disease, the composition comprising an engineered vector as described herein; and a pharmaceutically acceptable carrier.

In another aspect, described herein is a method of treating an ocular disease in a subject, the method comprising administering to the subject the engineered vector described herein or the pharmaceutical composition described herein.

In another aspect, described herein is a method of promoting cone survival in the retina of a subject, the method comprising intraocularly administering to the subject an effective amount of a composition comprising a vector comprising a nucleic acid construct comprising a retina-specific promoter operably linked to nucleic acid sequence encoding a transforming growth factor beta (TGF-β) polypeptide.

In yet another aspect, described herein is a method of promoting neuronal cell survival, the method comprising: delivering a TGF-P polypeptide to a microglial cell.

In another aspect, described herein is a method of treating a neurodegenerative disease or disorder in a subject in need thereof, the method comprising: administering to the subject a viral vector comprising a promoter active in a neuronal cell operatively linked to a nucleic acid sequence encoding a TGF-β polypeptide.

In one embodiment of this or any of the aspects described herein, the vector is selected from the group consisting of: an adeno-associated virus (AAV) vector; an adenovirus vector; and a lentiviral vector.

In another embodiment of any of the aspects, the AAV vector is selected from the group consisting of: serotype AAV8; AAV2; AAV5; AAV2/8, another AAV serotype as identified, for example, in Table 2 or Table 3. Table 2 lists non-limiting exemplary serotypes of AAV and accession numbers of the genome and capsid sequences that may be used in the methods and compositions described herein. Table 3 describes exemplary AAV serotypes and exemplary published corresponding capsid sequences that can be used as the AAV capsid in an rAAV vector as described herein. The AAV serotype is not limited to human AAV, but may, where appropriate, include non-human AAV, for example, avian or bovine AAV, as well as non-human primate AAV, examples of which are shown in Table 1.

In another embodiment of any of the aspects, the engineered vector comprises a regulatory element. In another embodiment of any of the aspects, the regulatory element is Woodchuck Hepatitis Virus (WHV) Posttranscriptional Regulatory Element (WPRE).

In another embodiment of any of the aspects, the TGF-β polypeptide is a TGF-β1, TGF-β2, or TGF-β3 polypeptide.

In another embodiment of any of the aspects, the retina-specific promoter is a red opsin promoter.

In another embodiment of any of the aspects, the pharmaceutical composition is formulated for delivery to the eye. In another embodiment of any of the aspects, the pharmaceutical composition is formulated for delivery to the retina. In another embodiment of any of the aspects, the pharmaceutical composition is formulated as an eye drop.

In another embodiment of any of the aspects, the pharmaceutically acceptable carrier is an ophthalmically acceptable vehicle.

In another embodiment of any of the aspects, the administering is selected from the group consisting of: intraocular injection, subretinal injection, retrobulbar injection, submacular injection, intravitreal injection, intrachoroidal injection, topical application, eye drops, and intraocular implantation.

In another embodiment of any of the aspects, the delivering comprises administering a vector encoding the TGF-β polypeptide to a neuronal cell associated with the microglial cell.

In another embodiment of any of the aspects, the delivering promotes signaling through a TGFBR1 and/or TGFBR2 receptor.

In another embodiment of any of the aspects, the subject has or is suspected of having a neurodegenerative disease or disorder or an ocular disease.

In another embodiment of any of the aspects, the subject is a mammal.

In another embodiment of any of the aspects, the subject is a human.

In another embodiment of any of the aspects, the ocular disease is a neurodegenerative ocular disease.

In another embodiment of any of the aspects, the ocular disease is selected from the group consisting of: retinitis pigmentosa; glaucoma; age-related macular degeneration; retinitis; sclerotic retinal maculodystrophy; diabetic retinopathy; proliferative retinopathy; toxic retinopathy; and retinopathy of prematurity.

In another embodiment of any of the aspects, the ocular disease is retinitis pigmentosa.

In another embodiment of any of the aspects, the neurodegenerative disease or disorder is selected from the group consisting of: retinitis pigmentosa; glaucoma; macular degeneration; retinitis; retinal maculodystrophy; diabetic retinopathy; Alzheimer’s disease, Parkinson’s disease, Huntington’s disease, amyotrophic lateral sclerosis (ALS), frontotemporal dementia, chronic traumatic encephalopathy (CTE), multiple sclerosis, and neuroinflammation.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in cell and molecular biology can be found in Molecular Biology of the Cell, W. W. Norton & Company; Sixth edition, 2014 (ISBN-10 : 9780815344322); Karp’s Cell and Molecular Biology, Wiley; 9th edition (2020) (ISBN-10 : 1119598249); The Merck Manual of Diagnosis and Therapy, 20th Edition, published by Merck Sharp & Dohme Corp., 2018 (ISBN 9780911910421, 0911910425); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), Taylor & Francis Limited, 2014 (ISBN 0815345305, 9780815345305); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “engineered” refers to a nucleic acid or vector as having been manipulated by the hand of man. For example, a vector is considered to be “engineered” when at least one aspect of the vector, e.g., gene expression or structure, has been manipulated by the hand of man to differ from the aspect as it might exist in nature.

The term “vector,” as used herein, refers to a nucleic acid construct designed for delivery to a host cell or for transfer between different host cells. The term “vector” encompasses any genetic element that is capable of replication when associated with the proper control elements, and that can transfer gene sequences to cells. A vector can include, but is not limited to, a cloning vector, an expression vector, a plasmid, phage, transposon, cosmid, chromosome, virus, virion, etc. A vector can be viral or non-viral.

As used herein, “expression vector” refers to a vector comprising a nucleic acid that includes an open reading frame (ORF) and nucleic acid regulatory elements or components necessary and sufficient to permit mRNA expression from the open reading frame. In particular, an expression vector is one that directs expression of a heterologous nucleic acid. The sequences expressed will often, but not necessarily, be heterologous to the cell. Expression vectors useful in the methods and compositions described herein can also include elements necessary for replication and propagation of the vector in a host cell. An expression vector can comprise additional elements, for example, the expression vector can have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing.

As used herein, the term “viral vector,” refers to a virus (e.g., AAV) particle that functions as a nucleic acid delivery vehicle, and which comprises the vector genome (e.g., viral DNA [vDNA]) packaged within a virion. Alternatively, in some contexts, the term “vector” may refer to the vector genome/vDNA alone. Viral vectors useful in the methods and compositions described herein can further be “targeted” virus vectors (e.g., having a directed tropism) and/or a “hybrid” parvovirus (i.e., in which the viral terminal repeats (TRs) and viral capsid are from different parvoviruses) as described in international patent publication WO 2000/28004 and Chao et al. (2000) Molecular Therapy 2:619.

As used herein the term “polynucleotide” refers to a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” polynucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

As used herein, by “isolate” or “purify” (or grammatical equivalents) in reference to a viral vector, it is meant that the viral vector is at least partially separated from at least some of the other components in the starting material. In representative embodiments an “isolated” or “purified” viral vector is enriched by at least about 10-fold, 100-fold, 1000-fold, 10,000-fold or more as compared with the starting material.

As used herein, the terms “protein” and “polypeptide” are used interchangeably to designate a polymer or series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a translated gene product and fragments thereof.

A” variant” amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

As used herein, the term “retina-specific promoter” refers to a nucleic acid regulatory element that directs the transcription of an operably linked nucleic acid sequence in a cell of the retina to a much greater degree than the operably-linked nucleic acid is transcribed in a non-retinal cell. In this context, “much greater degree” means at least 10-fold greater than in a non-retinal, non-neuronal cell or tissue, e.g., at least 20-fold, 30-fold, 40-fold, 50-fold or higher. Non-limiting examples of retina-specific promoters as the term is used herein include the rhodopsin kinase promoter, which is active in both rods and cones (see, e.g., Sun et al., Gene Ther. 17: 117-131 (2010)), and the opsin promoters (driving expression of S(blue), M (green) and L (red) opsin photopigment genes (see, e.g., Li et al., Vision Res. 48: 332-338 (2008)), which are active in cones.

As used herein, the term “microglia-specific promoter” or “microglial cell-specific promoter” refers to a nucleic acid regulatory element that directs the transcription of an operably linked nucleic acid sequence in a microglial cell to a much greater degree than the operably-linked nucleic acid is transcribed in a non-microglial cell. In this context, “much greater degree” means at least 10-fold greater than in a non-microglial cell or tissue, e.g., at least 20-fold, 30-fold, 40-fold, 50-fold or higher.

As used herein, the term “regulatory element” refers to a nucleic acid sequence recognized by the transcriptional or post-transcriptional machinery of a cell that influences the expression of a gene product. Transcriptional regulatory elements include, for example, promoters, enhancers, silencers, termination sequences, and other transcription factor binding sequences, among others. Post-transcriptional regulatory elements include, for example, elements that modulate or direct mRNA splicing, mRNA stability, polyadenylation, nuclear export, or processes such as viral or viral vector processing of viral genomic transcripts. In one embodiment, a regulatory element, e.g., a post-transcriptional regulatory element, includes a woodchuck hepatitis virus post-transcriptional regulatory element (WPRE; see, e.g., Higashimoto et al., Gene Ther. 14: 1298-1304 (2007).

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

As used herein, a “reference level” refers to a level of a biomarker in, for example, a normal, otherwise unaffected cell population or tissue (e.g., a biological sample obtained from a healthy subject, or a biological sample obtained from the subject at a prior time point, e.g., a biological sample obtained from a patient prior to being diagnosed with a neurodegenerative disease or disorder or an ocular disease, or a biological sample that has not been contacted with an engineered vector disclosed herein).

As used herein, an “appropriate control” refers to an untreated, otherwise identical cell or population (e.g., a patient who was not administered a pharmaceutical composition or engineered vector as described herein).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with an infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, but not limited to, administration of a vaccine which prevents infection or illness due to a pathogen). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### A BRIEF DESCRIPTION OF THE DRAWINGS

FIGS. 1A-1E demonstrates retinal expression of inflammatory genes after microglial depletion. (FIG. 1A) Timeline of microglial depletion. Microglia from FVB (rdl) mice were pharmacologically depleted with PLX5622 beginning at P20 with harvesting of retinas at P40. (FIG. 1B) Retinal cross-sections from P40 rd1 mice (n = 2) with or without depletion. Arrowheads depict IBA1-positive microglia in the ONL by immunostaining. Scale bar, 50 µm. (FIGS. 1C-1D) Representative gating (FIG. 1C) and quantification (FIG. 1D) by flow cytometry of microglia as a percentage of all retinal cells in P40 rd1 mice (n = 4) with or without depletion. Microglia were defined as CD11b-positive Ly6G/Ly6C-negative cells. For full gating strategy, see FIG. 5A. (FIG. 1E) mRNA expression of indicated genes in retinas (n = 4-5) from six- to eight-week-old WT (sighted FVB) or P40 rd1 mice with or without 20 days of PLX5622. Fold changes are relative to WT retinas. Data shown are mean ± SEM. * P<0.05, ** P<0.01, *** P<0.001, **** P<0.001 by two-tailed Student’s t-test for (FIG. 1D), two-tailed Student’s t-test with Bonferroni correction for (FIG. 1E). ONL, outer nuclear layer; INL, inner nuclear layer; GCL, ganglion cell layer; ns, not significant.

FIGS. 2A-2E shows the effect of overexpressing TGF-β isoforms on cone survival. (A, B) Schematics of AAV vector design (FIG. 2A) and delivery (FIG. 2B). (FIG. 2C) Representative flat-mounts of FVB (rd1) retinas treated with AAV8-GFP and harvested at P20 or P50. Paired images depict low and high magnifications (boxed areas). Scale bar, 1 mm. (FIG. 2D) Representative flat-mounts of rd1 retinas treated with indicated AAV vectors and harvested at P50. Scale bar, 1 mm. (FIG. 2E) Quantification of GFP-positive cones in central retinas of rd1 mice (n = 12-28) treated with indicated AAV vectors. Data shown are mean ± SEM. ** P<0.01, **** P<0.001 by two-tailed Student’s t-test with Bonferroni correction. ns, not significant.

FIGS. 3A-3E demonstrates the effect of AAV8-TGFB1 on long-term cone survival and cone-mediated vision. (FIGS. 3A, 3B) Representative flat-mounts of rd10 (FIG. 3A) and Rho^{-/-} (FIG. 3B) retinas treated with AAV8-GFP or AAV8-GFP plus AAV8-TGFB1. Paired images depict low and high magnifications (boxed areas). Scale bar, 1 mm. (FIG. 3C) Quantification of GFP-positive cones in central retinas of rd10 (n = 18) and Rho^{-/-} (n = 14) mice. (FIG. 3D) Percentage of time spent in dark in a 50:50 light-dark box for untreated animals (n = 8-10) and C3H (rdl) mice (n = 11-14) treated with AAV8-GFP or AAV8-GFP plus AAV8-TGFB1. (FIG. 3E) Visual acuity in eyes from P60 rd10 mice (n = 23) as measured by optomotor after treatment with AAV8-GFP or AAV8-GFP plus AAV8-TGFB1. Data shown are mean ± SEM. * P<0.05, ** P<0.01, *** P<0.001, **** P<0.001 by two-tailed Student’s t-test for (FIGS. 3C, 3E), two-tailed Student’s t-test with Bonferroni correction for (FIG. 3D).

FIGS. 4A-4G shows the role of retinal microglia in AAV8-TGFB1-mediated cone survival. (FIG. 4A) mRNA expression of indicated genes in FVB (rdl) retinas (n = 4-5) treated with AAV8-GFP or AAV8-GFP plus AAV8-TGFB1. Fold changes are relative to WT (sighted FVB) retinas. (FIGS. 4B, 4C) Representative images (FIG. 4B) and quantification (FIG. 4C) of IBA1-positive microglia in the ONL of P40 rdl retinas (n = 6-7) treated with AAV8-GFP or AAV8-GFP plus AAV8-TGFB1. Scale bar, 50 µm. (FIG. 4D) Volcano plot of up- and down-regulated genes in microglia sorted from P30 rd1 retinas (n = 7) after treatment with AAV8-GFP plus AAV8-TGFB1 relative to AAV8-GFP only. Dotted lines indicate adjusted P<0.05 and log2 fold-change >0.4. (FIG. 4E) Normalized RNA-seq counts for expression of Tgfbr1 and Tgfbr2 in microglia versus non-microglia cells from P30 rd1 retinas (n = 4-14). (FIG. 4F) Immunostaining for TGFBR2 in rd1;CX3CR1^{GFP/+} retinas (n = 2). Arrowheads indicate colocalization TGFBR2 with CX3CR1-positive microglia in the ONL. Scale bar, 10 µm. (FIG. 4G) Quantification of GFP-positive cones in central retinas of rd1 mice after 30 days of microglial depletion with PLX5622 (n = 16) or inhibition of TGFBR½ with LY364947 and SB431542 (n = 16). Data for untreated groups are taken from FIG. 2E. Data shown are mean ± SEM. * * * P<0.001, **** P<0.001 by two-tailed Student’s t-test with Bonferroni correction for (FIGS. 4A, 4G), two-tailed Student’s t-test for (FIG. 4C, FIG. 4E). INL, inner nuclear layer; ns, not significant.

FIG. 5A shows representative flow cytometry gating for microglia and non-microglia cells in the retina. Microglia were defined as CD11b-positive Ly6G/Ly6C-negative cells. Non-microglia were defined as CD11b-negative cells. FIG. 5B shows representative flow cytometry gating for lymphocytes, monocytes, and granulocytes from peripheral blood. Each population was defined based on its characteristic forward scatter (FSC) and side scatter (SSC) profile as previously described (1). FIG. 5C shows representative flow cytometry gating for peritoneal macrophages isolated from the peritoneal cavity. Peritoneal macrophages were defined as CD11b-positive F4/80-positive cells. FIGS. 5D-5E shows quantification of peripheral blood immune populations (n = 5-6) (FIG. 5D) and peritoneal macrophages (n = 6) (FIG. 5E) from P40 FVB (rdl) mice with or without 20 days of PLX5622. Data shown are mean ± SEM. ns, not significant by two-tailed Student’s t-test.

FIG. 6A shows the kinetics of GFP expression in cones after subretinal delivery of AAV8-GFP. Arrowheads indicate faint GFP expression. Scale bar, 50 µm. FIG. 6B shows mRNA expression of Tgfb1, Tgfb2, and Tgfb3 in FVB (rdl) retinas (n = 4-5) after treatment with AAV8-GFP plus AAV8-TGFB1, AAV8-TGFB2, or AAV8-TGFB3, respectively. Fold changes are relative to AAV8-GFP. FIG. 6C shows the quantification of TGFB1, TGFB2, and TGFB3 secreted during ex vivo culture from WT (CD-1) retinas (n = 6-7) treated with AAV8-GFP plus AAV8-TGFB1, AAV8-TGFB2, or AAV8-TGFB3, respectively. Data shown are mean ± SEM. ** P<0.01, *** P<0.001, **** P<0.001 by two-tailed Student’s t-test. RPE, retinal pigment epithelium; INL, inner nuclear layer; ns, not significant.

FIGS. 7A-7B show representative images (FIG. 7A) and quantification (FIG. 7B) of cone survival in central retinas of P20 rd1 mice (n = 9-13) treated with AAV8-GFP or AAV8-GFP plus AAV8-TGFB1. Scale bar, 500 µm. FIGS. 7C-7D show representative gating (FIG. 7C) and quantification (FIG. 7D) by flow cytometry of GFP-positive cones from P30 FVB (rdl) retinas (n = 11) treated with AAV8-GFP or AAV8-GFP plus AAV8-TGFB1. FIG. 7E shows immunostaining for cone arrestin (CAR) in flat-mounts of P50 rd1 retinas treated with AAV8-GFP or AAV8-GFP plus AAV8-TGFB1. Paired images depict low and high magnifications (boxed areas). Scale bar, 1 mm. FIG. 7F shows quantification of CAR-positive cones in central retinas of P50 rd1 mice (n = 12) treated with AAV8-GFP or AAV8-GFP plus AAV8-TGFB1. Data shown are mean ± SEM. ** P<0.01, **** P<0.001 by two-tailed Student’s t-test. ns, not significant.

FIG. 8A shows mRNA expression of Tgfb1 in Rho^{-/-} retinas (n = 4-5) after treatment with AAV8-GFP plus AAV8-TGFB1 relative to AAV8-GFP only. (FIGS. 8B, 8C) Representative cross-sections (FIG. 8B) and measurements of ONL thickness (FIG. 8C) at indicated distances from the optic nerve in P40 rd10 retinas (n = 6) treated with AAV8-GFP or AAV8-GFP plus AAV8-TGFB1. Scale bar, 50 µm. (FIG. 8D) Representative movement tracks during light-dark box testing from untreated animals (n = 8-10) and C3H (rdl) mice (n = 11-14) treated with AAV8-GFP or AAV8-GFP plus AAV8-TGFB1. Data shown are mean ± SEM. **** P<0.001 by two-tailed Student’s t-test. ns, not significant.

FIG. 9A shows representative images of the ocular fundus and lens from P30 WT (CD-1) eyes (n = 14) treated with AAV8-GFP or AAV8-GFP plus AAV8-TGFB1. Scale bar, 1 mm. (FIGS. 9B, 9C) Representative images (FIG. 9B) and quantification (FIG. 9C) of BRN3A-positive retinal ganglion cells (RGCs) in P30 WT retinas (n = 6) treated with AAV8-GFP or AAV8-GFP plus AAV8-TGFB1. RGCs were counted in four 20x fields per retina with the mean used to represent each sample. Scale bar, 100 µm. (FIG. 9D) Immunostaining for ZO-1, a component of epithelial tight junctions (2), in flat-mounted RPE preparations from P30 WT eyes (n = 4) treated with AAV8-GFP or AAV8-GFP plus AAV8-TGFB1. Scale bar, 100 µm. (FIG. 9E) Immunostaining for α-smooth muscle actin (α-SMA) in P30 WT eyes (n = 2-3) without treatment or treated with AAV8-GFP or AAV8-GFP plus AAV8-TGFB1. Arrowheads indicate α-SMA-positive cells in vessel walls. Scale bar, 50 µm. Data shown are mean ± SEM. ns, not significant; INL, inner nuclear layer.

FIGS. 10A-10B shows representative gating (FIG. 10A) and quantification (FIG. 10B) by flow cytometry of microglia as a percentage of all retinal cells in P40 FVB (rdl) retinas (n = 7-9) treated with AAV8-GFP or AAV8-GFP plus AAV8-TGFB1. (FIG. 10C) Normalized RNA-seq counts for indicated cell type markers in microglia sorted from P30 rd1 retinas (n = 14). (FIG. 10D) mRNA expression of Spp1 and Gas6 in sorted microglia from P30 rd1 retinas (n = 12) and P200 rd10 retinas (n = 10-12) after treatment with AAV8-GFP plus AAV8-TGFB1 relative to AAV8-GFP only. (FIG. 10E) Quantification by flow cytometry of retinal microglia from P35 rd1 retinas (n = 2-6) treated with AAV8-GFP or AAV8-GFP plus AAV8-TGFB1 with or without 15 days of PLX5622. Data shown are mean ± SEM. * P<0.05, *** P<0.001, **** P<0.001 by two-tailed Student’s t-test. RGC, retinal ganglion cell; ns, not significant.

### DETAILED DESCRIPTION

Described herein are compositions and methods for the treatment of neurodegenerative diseases. In particular, it has been discovered that the activation of TGF-β signaling through TGF-β receptors (TGFR) 1 and/or 2 can promote survival of neuronal cells through a mechanism involving microglia, a population of resident immune cells in the CNS. In various embodiments, the delivery of TGF-β polypeptides to the neuronal cell or tissue environment promotes survival of neuronal cells otherwise subject to cell death and neuronal degeneration. In some embodiments, the neuronal cells or tissue comprise ocular cells or tissue, including for example, retinal cells or tissue, including, for example, retinal photoreceptor cone cells. In some embodiments, the TGF-β activates signaling through TGF-β receptors expressed by microglia or microglial cells associated with the neuronal cells or tissue. The following provides various considerations relating to the practice of the various compositions and methods described.

### Neurodegenerative Diseases

In one aspect, described herein are methods and compositions for the treatment or prevention of a neurodegenerative disease or disorder. Neurodegenerative diseases can have varying etiologies; however, to the extent that a given neurodegenerative disease involves neuronal tissues comprising or associated with microglia, a population of resident immune cells in the CNS, the compositions and methods described herein for the delivery of TGF-β polypeptides are contemplated to provide therapeutic benefit. While not wishing to be bound by theory, in some instances it is contemplated that the action of TGF-β can suppress activation and secretion of inflammatory cytokines by the microglial cells, while in others, including the situation in several different models of retinitis pigmentosa as demonstrated herein, the beneficial effect of TGF-β, requires microglia, but does not necessarily modulate their release of inflammatory cytokines of factors. Thus, while many neurodegenerative diseases include an inflammatory component that may benefit from TGF-β delivery, those that do not, yet include a microglial component, can also benefit. Numerous neurodegenerative diseases and disorders are known in the art and can include, among others, ocular diseases such as retinitis pigmentosa; glaucoma; age-related macular degeneration; retinitis; sclerotic retinal maculodystrophy; diabetic retinopathy; proliferative retinopathy; toxic retinopathy; retinopathy of prematurity; and Parkinson’s disease, Huntington’s disease, Alzheimer’s disease, ALS, Multiple Sclerosis, and epilepsy, among others.

The molecular mechanisms of neuroinflammation and neurodegenerative diseases are further described, e.g., in Amor, S., et al. (2010), “Inflammation in neurodegenerative diseases.” Immunology, 129: 154-169; Barnham, K., Masters, C. & Bush, A. “Neurodegenerative diseases and oxidative stress.” Nat Rev Drug Discov 3, 205-214 (2004); the contents of each of which are incorporated herein by reference in their entireties.

As noted, one molecular mechanism for neurodegeneration involves excessive microglial activation, which results in the phagocytosis of neurons and release of pro-inflammatory cytokines. The cellular signaling of microglia in normal and neurodegenerative pathologies are described in detail, e.g., in Bin Liu and Jau-Shyong Hong. “Role of Microglia in Inflammation-Mediated Neurodegenerative Diseases: Mechanisms and Strategies for Therapeutic Intervention” Journal of Pharmacology and Experimental Therapeutics. Jan. 1, 2003, 304 (1) 1-7; Hickman, S., Izzy, S., Sen, P. et al. “Microglia in neurodegeneration.” Nat Neurosci 21, 1359-1369 (2018); Hickman, et. al., “Microglial dysfunction and defective beta-amyloid clearance pathways in aging Alzheimer’s disease mice.” J. Neurosci. 28, 8354-8360 (2008); Wojtera M, Sikorska B, Sobow T, Liberski PP. “Microglial cells in neurodegenerative disorders.” Folia Neuropathol. 2005;43(4):311-21. PMID: 16416395; Gehrmann J, Matsumoto Y, Kreutzberg GW. “Microglia: intrinsic immuneffector cell of the brain.” Brain Res Brain Res Rev. 1995 Mar;20(3):269-87; and Block ML, Zecca L, Hong J-S. “Microglia-mediated neurotoxicity: uncovering the molecular mechanisms.” Nat. Rev. Neurosci. 2007;8(1):57-69, the contents of each of which are incorporated herein by reference in their entireties.

An ocular disease or disorder as described herein includes any disease that affects vision or the eye. The ocular diseases described herein can affect an ocular region or site of the eye and/or neurons surrounding such regions, e.g., retina, choroid, sclera vitreous, vitreous chamber, optic nerve (i.e. the optic disc), and blood vessels and nerves which vascularize or innervate the ocular region or site. Ocular diseases and disorders are known in the art, and can include, but are not limited to: retinitis pigmentosa, maculopathies, acute macular neuroretinopathy; retinal degeneration, uveitis, Behcet’s disease; choroiditis, vascular diseases, exudative diseases, proliferative disorders, histoplasmosis, infectious disorders (e.g., fungal or viral infections), autoimmune encephalomyelitis, genetic disorders, tumors, trauma, retinal tears or holes, glaucoma; age-related macular degeneration; retinitis; sclerotic retinal maculodystrophy; choroideremia; diabetic retinopathy; proliferative retinopathy; toxic retinopathy; proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, and retinopathy of prematurity.

Some forms of degenerative ocular disease are caused by aberrant inflammation in the eye. For example, in retinitis pigmentosa, activated microglia in the retina phagocytose photoreceptors critical for the retina’s ability to respond to light. See, e.g., Zhao L et al. “Microglial phagocytosis of living photoreceptors contributes to inherited retinal degeneration.” EMBO Mol. Med. 2015;7(9):1179-1197; Peng B et al. Suppression of Microglial Activation Is Neuroprotective in a Mouse Model of Human Retinitis Pigmentosa. J. Neurosci. 2014;34(24):8139-8150.; and Smith JA, Das A, Ray SK, Banik NL. “Role of pro-inflammatory cytokines released from microglia in neurodegenerative diseases.” Brain Res. Bull. 2012;87(1):10-20, the contents of each of which are incorporated herein by reference in their entireties.

In some embodiments of any of the aspects described herein, the ocular disease is a retinal disease. The retina is a neuronal photoreceptor structure at the back of the eye. Specifically, the retina contains two major types of light-sensitive photoreceptor cells, rod cells and cone cells. Cone cells are responsible for color vision and require brighter light to function, as compared to rod cells. There are three types of cones, maximally sensitive to long- wavelength, medium- wavelength, and short-wavelength light (often referred to as red, green, and blue, respectively, though the sensitivity peaks are not actually at these colors). Cones are mostly concentrated in and near the fovea. Only a small percentage of photoreceptors are cones in the periphery of the retina. Objects are seen most sharply in focus when their images fall on the cone-enriched spot, as when one looks at an object directly. Cone cells and rods are connected through intermediate cells in the retina to nerve fibers of the optic nerve.

Reduced viability of cone cells is associated with various retinal disorders, e.g., retinitis pigmentosa.

In some embodiments of any of the aspects, the retinal disease or disorder is retinitis pigmentosa. Retinitis pigmentosa or “RP” is known in the art and encompasses a disparate group of genetic retinal degenerative disorders of rods and cones. Globally, the condition affects an estimated two million people, with thousands of pathogenic mutations identified to date spanning at least 80 different genes. Retinitis pigmentosa is often characterized by night blindness, progressive loss of peripheral vision, eventually leading to total blindness. In some cases, there can be a lack of pigmentation. Retinitis pigmentosa can be associated to degenerative opacity of the vitreous body, and cataract. Family history is prominent in retinitis pigmentosa; the pattern of inheritance may be autosomal recessive, autosomal dominant, or X-linked; the autosomal recessive form is the most common and can occur sporadically.

RP begins with the initial degeneration of rods which triggers secondary degeneration of cones, leading to significant loss of daylight, color, and high-acuity vision. RP generates ophthalmoscopic changes consisting of dark mosaic-like retinal pigmentation, attenuation of the retinal vessels, waxy pallor of the optic disc, and in the advanced forms, macular degeneration. Since cones are responsible for color and high acuity vision, it is their loss that leads to a significant reduction in the quality of life. In many cases, the disease-causing allele is expressed exclusively in rods; nonetheless, cone cell death follows rod cell death. See, e.g., Daiger SP, Sullivan LS, Bowne SJ. “Genes and mutations causing retinitis pigmentosa.” Clin. Genet. 2013;84(2):132-141; and Zhao L et al. “Microglial phagocytosis of living photoreceptors contributes to inherited retinal degeneration.” EMBO Mol. Med. 2015;7(9): 1179-1197, the contents of which is incorporated herein by reference in its entirety. Where the mutations affecting rods vary widely, and where the most life-altering effects stem from loss of cones, efforts are being made to promote cone survival despite loss of rods.

Provided herein in the working examples, is the discovery that delivery of different isoforms of transforming growth factor beta (TGF-β), an anti-inflammatory cytokine, in retinal cells can limit or halt cone cell degeneration in several different models of RP, and that the effect is dependent upon microglia. It is contemplated herein that other neurodegenerative diseases affecting neuronal tissue associated with microglia are also potential targets for therapy using a similar approach (e.g., administering to a subject an engineered vector or pharmaceutical composition providing or delivering TGF-β as described herein).

### Microglia

The immune system is involved in the normal function and injury repair of the central nervous system and this function is carried out by cells called microglia. Microglia are resident macrophages of the CNS that can sense changes in the microenvironment, help maintain neuronal function, and provide neuroprotection. Microglia are located throughout the brain, spinal cord, and in the retina of the eye. Microglia can be identified on the basis of various cell surface and intracellular markers. As a subset of macrophages, microglia share many markers with peripheral macrophages, including, for example, CD68, CD11b, Cx3cr1, and translocator protein (TSPO) but can be distinguished on the basis of location, i.e., the cells will be in association with neuronal tissues, as well as by the expression of more microglia-specific markers Transmembrane Protein 119 (TMEM119) and P2Y purinoceptor 12 (P2Y12 or P2YR12). The marker ionized calcium-binding adaptor molecule 1 (Iba1) is a macrophage marker also closely associated with the microglial cell sub-population.

Noxious insults in the retina or CNS such as oxidative stress, hypoxia or inherited mutations trigger microglia can reactivity manifested by amoeboid morphology, increased proliferation and migration of the cells to the sites of injury. Microglial activation includes a number of cellular responses, such as proliferation, increased or de-novo expression of immunomolecules, recruitment of immune cells to the site of injury and functional changes, e.g., the release of cytotoxic and/or inflammatory mediators. In addition, microglia have a strong antigen presenting function and a pronounced cytotoxic function.

Microglial activation results in their production of inflammatory cytokines such as IL-1, IL-6, and TNF-α. While release of these factors is apparently designed to prevent further damage to CNS tissue, they may also be toxic to neurons and other glial cells. As noted above and as discussed in the Examples herein, while it is clear that microglia are required for the TGF-β effect in rescue of cone cells in RP, this effect is not necessarily due to a suppression of microglial cell activation or microglial secretion of inflammatory cytokines; without wishing to be bound by theory, microglia appear to provide a survival-promoting environment for cone cells through one or more pathways involving TGF-β signaling.

Mouse models of retinitis pigmentosa have demonstrated that the microglia infiltrate and morphologically change during rod degeneration by translocating from the inner to the outer nuclear layer (ONL) of the retina. Microglia infiltrating the ONL of the retina acquire morphological features absent in inner retinal microglia, including: a redirection of processes to a predominantly radial orientation, extension of processes across the ONL to intercalate closely with photoreceptor somata, development of intracellular phagosomes in cellular processes, and acquisition of a rounded, amoeboid morphology containing multiple phagosomes. Microglia transiently upregulate phagocytic and lysosomal function upon ONL infiltration. The immunopositivity of microglial markers, e.g., CD68, and translocator protein (TSPO), indicates activation of microglia during infiltration of the ONL. When infiltrating microglia make contact with stressed but viable rod cells, the rods expose phosphatidylserine (PS) on their surface marking them for destruction by the immune cells. See, e.g., Smith JA, et al. “Role of pro-inflammatory cytokines released from microglia in neurodegenerative diseases.” Brain Res. Bull. 2012; 87(1):10-20, the contents of which is incorporated herein by reference in its entirety.

The morphology and function of microglial cells, e.g., identifying markers of active microglia (e.g, TSPO), can be monitored by methods known in the art. For example, microscopy, live cell imaging, confocal microscopy, immunochemistry, Western blotting, and ELISA assays can be used among others.

It is demonstrated in the Examples herein that the expression of a TGF-β polypeptide from a promoter active in cone cells can provide therapeutic benefit. Without wishing to be bound by theory, it is considered that the TGF-β expressed by viral vector infected cone cells acts upon associated microglial cells, which abundantly express TGF-β receptors, e.g., in a paracrine fashion. It is specifically contemplated that expression of a TGF-β polypeptide directly in the microglial cells, e.g., by expression from a vector including a TGF-β construct driven by a microglial cell-specific promoter could also provide benefits. It is specifically contemplated that such an autocrine approach would be beneficial in other, non-retinal, neurodegenerative conditions. If so desired, the skilled artisan can generate viral vectors including microglial cell-specific regulator elements driving TGF-β polypeptide expression for use in such approaches.

### TGF-β

As discussed herein above, the ectopic expression or delivery of Transforming Growth Factor-β polypeptides can be used to treat neurodegenerative diseases, including but not limited to neurodegenerative ocular diseases.

Transforming Growth Factor β, also referred to as TGF-β or TGF-beta, is a cytokine member of a large family of structurally related proteins in the so-called TGF-β superfamily. TGF-β has three mammalian isoforms, TGF-β1, TGF-β2 and TGF-β3, each of which can bind TGF-β receptor 2 (TGFBR2), which then recruits and phosphorylates TGF-β receptor 1 (TGFBR1, also known as Alk5). In the so-called canonical TGF-β pathway, phosphorylated TGFBR1 in turn phosphorylates downstream signaling molecules SMAD (mothers against decapentaplegic homolog) 2 and SMAD3, which then recruit SMAD4 and translocate to the nucleus to regulate transcription of TGF-β target genes. An alternative pathway involves TGF-β receptor activation of a variety of signal transduction kinases including, for example, the MAP kinases, ERK, P38, JNK, phosphatidylinositol 3 kinase or ROCK.

The polypeptide structures of the TGF-β isoforms are highly similar, with amino acid sequence homologies of approximately 70-80%. Each of the three isoforms is encoded as a larger precursor protein. The TGF-β1 precursor contains 390 amino acids and the TGF-β2 and TGF-β3 precursors each contain 412 amino acids. Each precursor has an N-terminal signal peptide of 20-30 amino acids, a pro-region referred to as latency associated peptide (LAP; also referred to as Pro-TGF-β ) and a 112-114 amino acid C-terminal region that becomes the mature TGF-β molecule following its proteolytic release from the pro-region. In some embodiments, TGF-β polypeptide expressed from a vector, e.g., a viral vector, as described herein is expressed as the mature form, i.e., without the LAP pro-peptide, but can include a signal peptide to ensure secretion. The mature TGF-β protein dimerizes to produce a 25 kDa active protein. TGF-β polypeptides include nine cysteine residues that are conserved among the family, eight of which form intramolecular disulfide bonds to create a so-called “cysteine knot” structure that is characteristic of the TGF-β superfamily. The ninth cysteine forms a disulfide bond with the ninth cysteine of another TGF-β molecule to produce a dimer. Other conserved residues are important for establishing secondary structure through hydrophobic interactions, but the region between the fifth and sixth conserved cysteines is the most divergent between different TGF-β proteins, exposed at the surface of the protein and implicated in receptor binding and specificity of TGF-β. The Examples provided herein demonstrate that TGF-b1 and TGF-b3 are effective to protect retinal cone cells from degradation in various models of retinitis pigmentosa. However, depending upon the neuronal tissue involved, it is contemplated that TGF-β2 and/or variants of the wild-type TGF-β1, TGF-β3 or TGF-β2 that bind and activate receptors can also be effective. The sequences of wild-type TGF-β1, 2 and 3 are known in the art and set out herein below. A variant will generally be at least 90% identical to one of the wild-type polypeptide isoforms, will include the conserved cysteine knot structure, and will bind to TGFBR2 in a manner that recruits TGFBR1. The crystal structure of TGFBR2 ligand binding domain has been solved at 1.1 A resolution - see, e.g., Boesen et al., Structure 10: 913-909 (2002), which is incorporated herein by reference in its entirety. The crystal structure of human TGF-β3 with the extracellular domain of the human receptor TGFBR2 has also been solved - see, e.g., Hart et al., Nat. Struct. Biol. 9: 203-208 (2002). The use of these or other structural coordinates for the TGF-β polypeptides and the TGFBR2 extracellular domain with in silico modeling software can permit the prediction of whether a given variant of a given TGF-β isoform will bind and activate TGFBR2 as required for activation of TGF-β signaling activity (measured, for example, by activation of one or more downstream signaling molecules or pathways, or, for example, by detection of TGF-β-regulated gene expression). Additional crystal structures of TGF-P polypeptides and their respective receptors are further described, e.g., in Mittl PR, Priestle JP, Cox DA, McMaster G, Cerletti N, Grütter MG. The crystal structure of TGF-beta 3 and comparison to TGF-beta 2: implications for receptor binding. Protein Sci. 1996;5(7):1261-1271. doi:10.1002/pro.5560050705; Shi M, Zhu J, Wang R, Chen X, Mi L, Walz T, Springer TA. “Latent TGF-P structure and activation.” Nature. 2011 Jun 15;474(7351):343-9. doi: 10.1038/nature10152. PMID: 21677751; PMCID: PMC4717672; Huse M, Chen YG, Massagué J, Kuriyan J. Crystal structure of the cytoplasmic domain of the type I TGF beta receptor in complex with FKBP12. Cell. 1999 Feb 5;96(3):425-36. doi: 10.1016/s0092-8674(00)80555-3. PMID: 10025408; Daopin S, Piez KA, Ogawa Y, Davies DR. “Crystal structure of transforming growth factor-beta 2: an unusual fold for the superfamily.” Science. 1992 Jul 17;257(5068):369-73. doi: 10.1126/science.1631557. PMID: 1631557; and Radaev S, Zou Z, Huang T, Lafer EM, Hinck AP, Sun PD. “Ternary complex of transforming growth factor-beta1 reveals isoform-specific ligand recognition and receptor recruitment in the superfamily.” J Biol Chem. 2010 May 7;285(19):14806-14. doi: 10.1074/jbc.M109.079921. Epub 2010 Mar 5. PMID: 20207738; PMCID: PMC2863181, the contents of each of which are incorporated herein by reference in their entireties, and provide additional guidance regarding conserved amino acid residues of TGF-P polypeptides and structure necessary for and/or influencing functional receptor binding.

Non-limiting examples of TGF-β target genes activated by TGF-β include 4EBP1, and the cyclin-dependent kinase inhibitors P15, P21 and P57. Non-limiting examples of TGF-β target genes the expression of which is suppressed by TGF-β include CDC25A, E2F-1, Bcl-2, TGF-α and c-Myc.

The type 1 and 2 receptors in TGF-P superfamily share a common three-finger toxin fold, yet have distinct binding sites, and do not appear to cross-react. Structural studies of the TGF-P receptor extracellular domains complexed to their cognate ligands have revealed the similarities and differences in conformation between isoforms. For example, Radaev et al. (2010), describe how the type I receptor contacts both monomers of TGF-β1, generating two primarily hydrophobic patches of the interface. The interface between TGF-β1_{A} and TGFβR1 consists of Trp³⁰, Trp³², Tyr⁹⁰, and Leu¹⁰¹ of the “palm” side of TGF-β1_{A} fingers and Ile⁵⁴, Pro⁵⁵, and Phe⁶⁰ from TGFβR1. This interface is well conserved in the structure of TGF-β3 ternary complex as well as among the sequences of three TGF-P isoforms. However, one hydrophobic interaction between Thr⁶⁷ of TGF-β3_{B} and Val⁷¹ of TGFβR1, is absent in the TGF-β1 complex because of a partial disorder of β4-β5 loop around Val⁷¹ of TGFβR1. The interactions between TGF-β1 and TGFβR2 involve five TGF-β1 residues (Arg²⁵, His³⁴, Tyr⁹¹, Gly⁹³, and Arg⁹⁴) at the tips of its fingers and seven TGFβR2 residues (Phe³⁰, Asp³², Ser⁴⁹, Ile⁵⁰, Ser⁵², Ile⁵³, and Glu¹¹⁹) on the base of the toxin-fold fingers of the receptor. See, e.g., Radaev S, Zou Z, Huang T, Lafer EM, Hinck AP, Sun PD. Ternary complex of transforming growth factor-beta1 reveals isoform-specific ligand recognition and receptor recruitment in the superfamily. J Biol Chem. 2010;285(19):14806-14814. doi:10.1074/jbc.M109.079921, which is incorporated herein by reference in its entirety.

The polynucleotide and amino acid sequences for human and non-human TGF-β polypeptides and receptors are known in the art, e.g., TGF-β1 NCBI Gene IDs: 7040, 21803, 59086, 282089, 403998, 768263; TGF-β2 NCBI Gene IDs: 7042, 21808, 81809, 534069, 488596; TGF-β3 NCBI Gene IDs: 7043, 21809, 25717, 453054, 538957, 101098469; TGF-β receptor 1 NCBI Gene IDs: 7046, 21812, 29591, 282382, 472992, 100034117, 101094057; and TGF-β receptor 2 NCBI Gene IDs: 7048, 81810, 21813, 477039, 535376, 703088, 100033860, 100718284.

Exemplary coding and amino acid sequences of TGF-β polypeptides as described herein are known in the art and provided below.

### Human TGFβ Isoform Coding Sequences

### Human TGFB1 Coding Sequence-Homo Sapiens Transforming Growth Factor Beta 1 (TGFB1), mRNA, NCBI Sequence ID: NM_000660.7

ATGCCGCCCTCCGGGCTGCGGCTGCTGCCGCTGCTGCTACCGCTGCTGTG
GCTACTGGTGCTGACGCCTGGCCGGCCGGCCGCGGGACTATCCACCTGCA
AGACTATCGACATGGAGCTGGTGAAGCGGAAGCGCATCGAGGCCATCCGC
GGCCAGATCCTGTCCAAGCTGCGGCTCGCCAGCCCCCCGAGCCAGGGGGA
GGTGCCGCCCGGCCCGCTGCCCGAGGCCGTGCTCGCCCTGTACAACAGCA
CCCGCGACCGGGTGGCCGGGGAGAGTGCAGAACCGGAGCCCGAGCCTGAG
GCCGACTACTACGCCAAGGAGGTCACCCGCGTGCTAATGGTGGAAACCCA
CAACGAAATCTATGACAAGTTCAAGCAGAGTACACACAGCATATATATGT
TCTTCAACACATCAGAGCTCCGAGAAGCGGTACCTGAACCCGTGTTGCTC
TCCCGGGCAGAGCTGCGTCTGCTGAGGCTCAAGTTAAAAGTGGAGCAGCA
CGTGGAGCTGTACCAGAAATACAGCAACAATTCCTGGCGATACCTCAGCA
ACCGGCTGCTGGCACCCAGCGACTCGCCAGAGTGGTTATCTTTTGATGTC
ACCGGAGTTGTGCGGCAGTGGTTGAGCCGTGGAGGGGAAATTGAGGGCTT
TCGCCTTAGCGCCCACTGCTCCTGTGACAGCAGGGATAACACACTGCAAG
TGGACATCAACGGGTTCACTACCGGCCGCCGAGGTGACCTGGCCACCATT
CATGGCATGAACCGGCCTTTCCTGCTTCTCATGGCCACCCCGCTGGAGAG
GGCCCAGCATCTGCAAAGCTCCCGGCACCGCCGAGCCCTGGACACCAACT
ATTGCTTCAGCTCCACGGAGAAGAACTGCTGCGTGCGGCAGCTGTACATT
GACTTCCGCAAGGACCTCGGCTGGAAGTGGATCCACGAGCCCAAGGGCTA
CCATGCCAACTTCTGCCTCGGGCCCTGCCCCTACATTTGGAGCCTGGACA
CGCAGTACAGCAAGGTCCTGGCCCTGTACAACCAGCATAACCCGGGCGCC
TCGGCGGCGCCGTGCTGCGTGCCGCAGGCGCTGGAGCCGCTGCCCATCGT
GTACTACGTGGGCCGCAAGCCCAAGGTGGAGCAGCTGTCCAACATGATCG
TGCGCTCCTGCAAGTGCAGCTG (SEQ ID NO: 1)

### Human TGFB2 Variant 1 Coding Sequence- Homo Sapiens Transforming Growth Factor Beta 2 (TGFB2), Transcript Variant 1, mRNA, NCBI Sequence ID: NM_001135599.4

ATGCACTACTGTGTGCTGAGCGCTTTTCTGATCCTGCATCTGGTCACGGT
CGCGCTCAGCCTGTCTACCTGCAGCACACTCGATATGGACCAGTTCATGC
GCAAGAGGATCGAGGCGATCCGCGGGCAGATCCTGAGCAAGCTGAAGCTC
ACCAGTCCCCCAGAAGACTATCCTGAGCCCGAGGAAGTCCCCCCGGAGGT
GATTTCCATCTACAACAGCACCAGGGACTTGCTCCAGGAGAAGGCGAGCC
GGAGGGCGGCCGCCTGCGAGCGCGAGAGGAGCGACGAAGAGTACTACGCC
AAGGAGGTTTACAAAATAGACATGCCGCCCTTCTTCCCCTCCGAAACTGT
CTGCCCAGTTGTTACAACACCCTCTGGCTCAGTGGGCAGCTTGTGCTCCA
GACAGTCCCAGGTGCTCTGTGGGTACCTTGATGCCATCCCGCCCACTTTC
TACAGACCCTACTTCAGAATTGTTCGATTTGACGTCTCAGCAATGGAGAA
GAATGCTTCCAATTTGGTGAAAGCAGAGTTCAGAGTCTTTCGTTTGCAGA
ACCCAAAAGCCAGAGTGCCTGAACAACGGATTGAGCTATATCAGATTCTC
AAGTCCAAAGATTTAACATCTCCAACCCAGCGCTACATCGACAGCAAAGT
TGTGAAAACAAGAGCAGAAGGCGAATGGCTCTCCTTCGATGTAACTGATG
CTGTTCATGAATGGCTTCACCATAAAGACAGGAACCTGGGATTTAAAATA
AGCTTACACTGTCCCTGCTGCACTTTTGTACCATCTAATAATTACATCAT
CCCAAATAAAAGTGAAGAACTAGAAGCAAGATTTGCAGGTATTGATGGCA
CCTCCACATATACCAGTGGTGATCAGAAAACTATAAAGTCCACTAGGAAA
AAAAACAGTGGGAAGACCCCACATCTCCTGCTAATGTTATTGCCCTCCTA
CAGACTTGAGTCACAACAGACCAACCGGCGGAAGAAGCGTGCTTTGGATG
CGGCCTATTGCTTTAGAAATGTGCAGGATAATTGCTGCCTACGTCCACTT
TACATTGATTTCAAGAGGGATCTAGGGTGGAAATGGATACACGAACCCAA
AGGGTACAATGCCAACTTCTGTGCTGGAGCATGCCCGTATTTATGGAGTT
CAGACACTCAGCACAGCAGGGTCCTGAGCTTATATAATACCATAAATCCA
GAAGCATCTGCTTCTCCTTGCTGCGTGTCCCAAGATTTAGAACCTCTAAC
CATTCTCTACTACATTGGCAAAACACCCAAGATTGAACAGCTTTCTAATA
TGATTGTAAAGTCTTGCAAATGCAGCTAA (SEQ ID NO:2)

### Human TGFB2 Variant 2 Coding Sequence- Homo Sapiens Transforming Growth Factor Beta 2 (TGFB2), Transcript Variant 2, mRNA, NCBI Sequence ID: NM_003238.6

ATGCACTACTGTGTGCTGAGCGCTTTTCTGATCCTGCATCTGGTCACGGT
CGCGCTCAGCCTGTCTACCTGCAGCACACTCGATATGGACCAGTTCATGC
GCAAGAGGATCGAGGCGATCCGCGGGCAGATCCTGAGCAAGCTGAAGCTC
ACCAGTCCCCCAGAAGACTATCCTGAGCCCGAGGAAGTCCCCCCGGAGGT
GATTTCCATCTACAACAGCACCAGGGACTTGCTCCAGGAGAAGGCGAGCC
GGAGGGCGGCCGCCTGCGAGCGCGAGAGGAGCGACGAAGAGTACTACGCC
AAGGAGGTTTACAAAATAGACATGCCGCCCTTCTTCCCCTCCGAAAATGC
CATCCCGCCCACTTTCTACAGACCCTACTTCAGAATTGTTCGATTTGACG
TCTCAGCAATGGAGAAGAATGCTTCCAATTTGGTGAAAGCAGAGTTCAGA
GTCTTTCGTTTGCAGAACCCAAAAGCCAGAGTGCCTGAACAACGGATTGA
GCTATATCAGATTCTCAAGTCCAAAGATTTAACATCTCCAACCCAGCGCT
ACATCGACAGCAAAGTTGTGAAAACAAGAGCAGAAGGCGAATGGCTCTCC
TTCGATGTAACTGATGCTGTTCATGAATGGCTTCACCATAAAGACAGGAA
CCTGGGATTTAAAATAAGCTTACACTGTCCCTGCTGCACTTTTGTACCAT
CTAATAATTACATCATCCCAAATAAAAGTGAAGAACTAGAAGCAAGATTT
GCAGGTATTGATGGCACCTCCACATATACCAGTGGTGATCAGAAAACTAT
AAAGTCCACTAGGAAAAAAAACAGTGGGAAGACCCCACATCTCCTGCTAA
TGTTATTGCCCTCCTACAGACTTGAGTCACAACAGACCAACCGGCGGAAG
AAGCGTGCTTTGGATGCGGCCTATTGCTTTAGAAATGTGCAGGATAATTG
CTGCCTACGTCCACTTTACATTGATTTCAAGAGGGATCTAGGGTGGAAAT
GGATACACGAACCCAAAGGGTACAATGCCAACTTCTGTGCTGGAGCATGC
CCGTATTTATGGAGTTCAGACACTCAGCACAGCAGGGTCCTGAGCTTATA
TAATACCATAAATCCAGAAGCATCTGCTTCTCCTTGCTGCGTGTCCCAAG
ATTTAGAACCTCTAACCATTCTCTACTACATTGGCAAAACACCCAAGATT
GAACAGCTTTCTAATATGATTGTAAAGTCTTGCAAATGCAGCTAA (SEQ ID NO: 3)

### Human TGFB3 Variant 1 Coding Sequence- Homo Sapiens Transforming Growth Factor Beta 3 (TGFB3), Transcript Variant 1, mRNA, Sequence ID: NM_{_}003239.5

ATGAAGATGCACTTGCAAAGGGCTCTGGTGGTCCTGGCCCTGCTGAACTT
TGCCACGGTCAGCCTCTCTCTGTCCACTTGCACCACCTTGGACTTCGGCC
ACATCAAGAAGAAGAGGGTGGAAGCCATTAGGGGACAGATCTTGAGCAAG
CTCAGGCTCACCAGCCCCCCTGAGCCAACGGTGATGACCCACGTCCCCTA
TCAGGTCCTGGCCCTTTACAACAGCACCCGGGAGCTGCTGGAGGAGATGC
ATGGGGAGAGGGAGGAAGGCTGCACCCAGGAAAACACCGAGTCGGAATAC
TATGCCAAAGAAATCCATAAATTCGACATGATCCAGGGGCTGGCGGAGCA
CAACGAACTGGCTGTCTGCCCTAAAGGAATTACCTCCAAGGTTTTCCGCT
TCAATGTGTCCTCAGTGGAGAAAAATAGAACCAACCTATTCCGAGCAGAA
TTCCGGGTCTTGCGGGTGCCCAACCCCAGCTCTAAGCGGAATGAGCAGAG
GATCGAGCTCTTCCAGATCCTTCGGCCAGATGAGCACATTGCCAAACAGC
GCTATATCGGTGGCAAGAATCTGCCCACACGGGGCACTGCCGAGTGGCTG
TCCTTTGATGTCACTGACACTGTGCGTGAGTGGCTGTTGAGAAGAGAGTC
CAACTTAGGTCTAGAAATCAGCATTCACTGTCCATGTCACACCTTTCAGC
CCAATGGAGATATCCTGGAAAACATTCACGAGGTGATGGAAATCAAATTC
AAAGGCGTGGACAATGAGGATGACCATGGCCGTGGAGATCTGGGGCGCCT
CAAGAAGCAGAAGGATCACCACAACCCTCATCTAATCCTCATGATGATTC
CCCCACACCGGCTCGACAACCCGGGCCAGGGGGGTCAGAGGAAGAAGCGG
GCTTTGGACACCAATTACTGCTTCCGCAACTTGGAGGAGAACTGCTGTGT
GCGCCCCCTCTACATTGACTTCCGACAGGATCTGGGCTGGAAGTGGGTCC
ATGAACCTAAGGGCTACTATGCCAACTTCTGCTCAGGCCCTTGCCCATAC
CTCCGCAGTGCAGACACAACCCACAGCACGGTGCTGGGACTGTACAACAC
TCTGAACCCTGAAGCATCTGCCTCGCCTTGCTGCGTGCCCCAGGACCTGG
AGCCCCTGACCATCCTGTACTATGTTGGGAGGACCCCCAAAGTGGAGCAG
CTCTCCAACATGGTGGTGAAGTCTTGTAAATGTAGCTGA (SEQ ID NO : 4)

### Human TGFB3 Variant 2 Coding Sequence- Homo Sapiens Transforming Growth Factor Beta 3 (TGFB3), Transcript Variant 3, mRNA, Sequence ID: NM_001329938.2

ATGAAGATGCACTTGCAAAGGGCTCTGGTGGTCCTGGCCCTGCTGAACTT
TGCCACGGTCAGCCTCTCTCTGTCCACTTGCACCACCTTGGACTTCGGCC
ACATCAAGAAGAAGAGGGTGGAAGCCATTAGGGGACAGATCTTGAGCAAG
CTCAGGCTCACCAGCCCCCCTGAGCCAACGGTGATGACCCACGTCCCCTA
TCAGGTCCTGGCCCTTTACAACAGCACCCGGGAGCTGCTGGAGGAGATGC
ATGGGGAGAGGGAGGAAGGCTGCACCCAGGAAAACACCGAGTCGGAATAC
TATGCCAAAGAAATCCATAAATTCGACATGATCCAGGGGCTGGCGGAGCA
CAACGAACTGGCTGTCTGCCCTAAAGGAATTACCTCCAAGGTTTTCCGCT
TCAATGTGTCCTCAGTGGAGAAAAATAGAACCAACCTATTCCGAGCAGAA
TTCCGGGTCTTGCGGGTGCCCAACCCCAGCTCTAAGCGGAATGAGCAGAG
GATCGAGCTCTTCCAGATCCTTCGGCCAGATGAGCACATTGCCAAACAGC
GCTATATCGGTGGCAAGAATCTGCCCACACGGGGCACTGCCGAGTGGCTG
TCCTTTGATGTCACTGACACTGTGCGTGAGTGGCTGTTGAGAAGAGAGTC
CAACTTAGGTCTAGAAATCAGCATTCACTGTCCATGTCACACCTTTCAGC
CCAATGGAGATATCCTGGAAAACATTCACGAGGTGATGGAAATCAAATTC
AAAGGCGTGGACAATGAGGATGACCATGGCCGTGGAGATCTGGGGCGCCT
CAAGAAGCAGAAGGATCACCACAACCCTCATCTAATCCTCATGATGATTC
CCCCACACCGGCTCGACAACCCGGGCCAGGGGGGTCAGAGGAAGAAGCGG
GCTTTGGACACCAATTACTGCTTCCGGTGA (SEQ IDNO: 5)

### Human Tgfβ Isoform Amino Acid Sequences

### Transforming Growth Factor Beta-1 Proprotein Preproprotein [Homo Sapiens], NCBI Reference Sequence: NP_000651.3

MPPSGLRLLPLLLPLLWLLVLTPGRPAAGLSTCKTIDMELVKRKRIEAIR
GQILSKLRLASPPSQGEVPPGPLPEAVLALYNSTRDRVAGESAEPEPEPE
ADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDV
TGVVRQWLSRGGEIEGFRLSAHCSCDSRDNTLQVDINGFTTGRRGDLATI
HGMNRPFLLLMATPLERAQHLQSSRHRRALDTNYCFSSTEKNCCVRQLYI
DFRKDLGWKWIHEPKGYHANFCLGPCPYIWSLDTQYSKVLALYNQHNPGA
SAAPCCVPQALEPLPIVYYVGRKPKVEQLSNMIVRSCKCS (SEQ ID N O: 6)

### Transforming Growth Factor Beta-2 Proprotein Isoform 1 Precursor [Homo Sapiens], NCBI Reference Sequence: NP_001129071.1

MHYCVLSAFLILHLVTVALSLSTCSTLDMDQFMRKRIEAIRGQILSKLKL
TSPPEDYPEPEEVPPEVISIYNSTRDLLQEKASRRAAACERERSDEEYYA
KEVYKIDMPPFFPSETVCPWTTPSGSVGSLCSRQSQVLCGYLDAIPPTFY
RPYFRIVRFDVSAMEKNASNLVKAEFRVFRLQNPKARVPEQRIELYQILK
SKDLTSPTQRYIDSKVVKTRAEGEWLSFDVTDAVHEWLHHKDRNLGFKIS
LHCPCCTFVPSNNYIIPNKSEELEARFAGIDGTSTYTSGDQKTIKSTRKK
NSGKTPHLLLMLLPSYRLESQQTNRRKKRALDAAYCFRNVQDNCCLRPLY
IDFKRDLGWKWIHEPKGYNANFCAGACPYLWSSDTQHSRVLSLYNTINPE
ASASPCCVSQDLEPLTILYYIGKTPKIEQLSNMIVKSCKCS (SEQ ID NO: 7)

### Transforming Growth Factor Beta-2 Proprotein Isoform 2 Preproprotein [Homo Sapiens], NCBI Reference Sequence: NP_003229.1

MHYCVLSAFLILHLVTVALSLSTCSTLDMDQFMRKRIEAIRGQILSKLKL
TSPPEDYPEPEEVPPEVISIYNSTRDLLQEKASRRAAACERERSDEEYYA
KEVYKIDMPPFFPSENAIPPTFYRPYFRIVRFDVSAMEKNASNLVKAEFR
VFRLQNPKARVPEQRIELYQILKSKDLTSPTQRYIDSKVVKTRAEGEWLS
FDVTDAVHEWLHHKDRNLGFKISLHCPCCTFVPSNNYIIPNKSEELEARF
AGIDGTSTYTSGDQKTIKSTRKKNSGKTPHLLLMLLPSYRLESQQTNRRK
KRALDAAYCFRNVQDNCCLRPLYIDFKRDLGWKWIHEPKGYNANFCAGAC
PYLWSSDTQHSRVLSLYNTINPEASASPCCVSQDLEPLTILYYIGKTPKI
EQLSNMIVKSCKCS (SEQ ID NO: 8)

### Transforming Growth Factor Beta-3 Proprotein Isoform 1 Preproprotein [Homo Sapiens], NCBI Reference Sequence: NP_003230.1

MKMHLQRALVVLALLNFATVSLSLSTCTTLDFGHIKKKRVEAIRGQILSK
LRLTSPPEPTVMTHVPYQVLALYNSTRELLEEMHGEREEGCTQENTESEY
YAKEIHKFDMIQGLAEHNELAVCPKGITSKVFRFNVSSVEKNRTNLFRAE
FRVLRVPNPSSKRNEQRIELFQILRPDEHIAKQRYIGGKNLPTRGTAEWL
SFDVTDTVREWLLRRESNLGLEISIHCPCHTFQPNGDILENIHEVMEIKF
KGVDNEDDHGRGDLGRLKKQKDHHNPHLILMMIPPHRLDNPGQGGQRKKR
ALDTNYCFRNLEENCCVRPLYIDFRQDLGWKWVHEPKGYYANFCSGPCPY
LRSADTTHSTVLGLYNTLNPEASASPCCVPQDLEPLTILYYVGRTPKVEQ
LSNMWKSCKCS (SEQ ID NO: 9)

### Transforming Growth Factor Beta-3 Proprotein Isoform 2 Precursor [Homo Sapiens], NCBI Reference Sequence: NP_001316867.1

MKMHLQRALVVLALLNFATVSLSLSTCTTLDFGHIKKKRVEAIRGQILSK
LRLTSPPEPTVMTHVPYQVLALYNSTRELLEEMHGEREEGCTQENTESEY
YAKEIHKFDMIQGLAEHNELAVCPKGITSKVFRFNVSSVEKNRTNLFRAE
FRVLRVPNPSSKRNEQRIELFQILRPDEHIAKQRYIGGKNLPTRGTAEWL
SFDVTDTVREWLLRRESNLGLEISIHCPCHTFQPNGDILENIHEVMEIKF
KGVDNEDDHGRGDLGRLKKQKDHHNPHLILMMIPPHRLDNPGQGGQRKKR
ALDTNYCFR (SEQ ID NO: 10)

### Mouse TGFβ Isoform Coding Sequences

### Mus Musculus Transforming Growth Factor, Beta 1 (Tgfb1), mRNA, NCBI Reference Sequence ID: NM_011577.2; See Also, NCBI Reference No. GenBank: BC013738.1- Mus Musculus Transforming Growth Factor, Beta 1, mRNA (cDNA Clone MGC:5747 IMAGE:3586216), Complete Cds

atgccgccctcggggctgcggctactgccgcttctgctcccactcccgtg
gcttctagtgctgacgcccgggaggccagccgcgggactctccacctgca
agaccatcgacatggagctggtgaaacggaagcgcatcgaagccatccgt
ggccagatcctgtccaaactaaggctcgccagtcccccaagccaggggga
ggtaccgcccggcccgctgcccgaggcggtgctcgctttgtacaacagca
cccgcgaccgggtggcaggcgagagcgccgacccagagccggagcccgaa
gcggactactatgctaaagaggtcacccgcgtgctaatggtggaccgcaa
caacgccatctatgagaaaaccaaagacatctcacacagtatatatatgt
tcttcaatacgtcagacattcgggaagcagtgcccgaacccccattgctg
tcccgtgcagagctgcgcttgcagagattaaaatcaagtgtggagcaaca
tgtggaactctaccagaaatatagcaacaattcctggcgttaccttggta
accggctgctgacccccactgatacgcctgagtggctgtcttttgacgtc
actggagttgtacggcagtggctgaaccaaggagacggaatacagggctt
tcgattcagcgctcactgctcttgtgacagcaaagataacaaactccacg
tggaaatcaacgggatcagccccaaacgtcggggcgacctgggcaccatc
catgacatgaaccggcccttcctgctcctcatggccacccccctggaaag
ggcccagcacctgcacagctcacggcaccggagagccctggataccaact
attgcttcagctccacagagaagaactgctgtgtgcggcagctgtacatt
gactttaggaaggacctgggttggaagtggatccacgagcccaagggcta
ccatgccaacttctgtctgggaccctgcccctatatttggagcctggaca
cacagtacagcaaggtccttgccctctacaaccaacacaacccgggcgct
tcggcgtcaccgtgctgcgtgccgcaggctttggagccactgcccatcgt
ctactacgtgggtcgcaagcccaaggtggagcagttgtccaacatgattg
tgcgctcctgcaagtgcagctga (SEQ ID NO: 11)

### Mus Musculus Transforming Growth Factor, Beta 2 (Tgfb2), Transcript Variant 1, mRNA, NCBI Reference Sequence ID: NM 009367.4

atgcactactgtgtgctgagcacctttttgctcctgcatctggtcccggt
ggcgctcagtctgtctacctgcagcaccctcgacatggatcagtttatgc
gcaagaggatcgaggccatccgcgggcagatcctgagcaagctgaagctc
accagccccccggaagactatccggagccggatgaggtccccccggaggt
gatttccatctacaacagtaccagggacttactgcaggagaaggcaagcc
ggagggcagccgcctgcgagcgcgagcggagcgacgaggagtactacgcc
aaggaggtttataaaatcgacatgccgtcccacctcccctccgaaaatgc
catcccgcccactttctacagaccctacttcagaatcgtccgctttgatg
tctcaacaatggagaaaaatgcttcgaatctggtgaaggcagagttcagg
gtcttccgcttgcaaaaccccaaagccagagtggccgagcagcggattga
actgtatcagatccttaaatccaaagacttaacatctcccacccagcgct
acatcgatagcaaggttgtgaaaaccagagcggagggtgaatggctctcc
ttcgacgtgacagacgctgtgcaggagtggcttcaccacaaagacaggaa
cctggggtttaaaataagtttacactgcccctgctgtaccttcgtgccgt
ctaataattacatcatcccgaataaaagcgaagagctcgaggcgagattt
gcaggtattgatggcacctctacatatgccagtggtgatcagaaaactat
aaagtccactaggaaaaaaaccagtgggaagaccccacatctcctgctaa
tgttgttgccctcctacagactggagtcacaacagtccagccggcggaag
aagcgcgctttggatgctgcctactgctttagaaatgtgcaggataattg
ctgccttcgccctctttacattgattttaagagggatcttggatggaaat
ggatccatgaacccaaagggtacaatgctaacttctgtgctggggcatgc
ccatatctatggagttcagacactcaacacaccaaagtcctcagcctgta
caacaccataaatcccgaagcttccgcttccccttgctgtgtgtcccagg
atctggaaccactgaccattctctattacattggaaatacgcccaagatc
gaacagctttccaatatgattgtcaagtcttgtaaatgcagctaa (SEQ ID NO: 12)

### Mus Musculus Transforming Growth Factor, Beta 3 (Tgfb3), mRNA, NCBI Reference Sequence ID: NM_009368.3

atgaagatgcacttgcaaagggctctggtagtcctggccctgctgaactt
ggccacaatcagcctctctctgtccacttgcaccacgttggacttcggcc
acatcaagaagaagagggtggaagccattaggggacagatcttgagcaag
ctcaggctcaccagcccccctgagccatcggtgatgacccacgtccccta
tcaggtcctggcactttacaacagcacccgggagttgctggaagagatgc
acggggagagggaggaaggctgcactcaggagacctcggagtctgagtac
tatgccaaagagatccataaattcgacatgatccagggactggcggagca
caatgaactggccgtctgccccaaaggaattacctctaaggtttttcgtt
tcaatgtgtcctcagtggagaaaaatggaaccaatctgttccgggcagag
ttccgggtcttgcgggtgcccaaccccagctccaagcgcacagagcagag
aattgagctcttccagatacttcgaccggatgagcacatagccaagcagc
gctacataggtggcaagaatctgcccacaaggggcaccgctgaatggctg
tctttcgatgtcactgacactgtgcgcgagtggctgttgaggagagagtc
caacttgggtctggaaatcagcatccactgtccatgtcacacctttcagc
ccaatggagacatactggaaaatgttcatgaggtgatggaaatcaaattc
aaaggagtggacaatgaagatgaccatggccgtggagacctggggcgtct
caagaagcaaaaggatcaccacaacccacacctgatcctcatgatgatcc
ccccacaccgactggacagcccaggccagggcagtcagaggaagaagagg
gccctggacaccaattactgcttccgcaacctggaggagaactgctgtgt
acgccccctttatattgacttccggcaggatctaggctggaaatgggtcc
acgaacctaagggttactatgccaacttctgctcaggcccttgcccatac
ctccgcagcgcagacacaacccatagcacggtgcttggactatacaacac
cctgaacccagaggcgtctgcctcgccatgctgcgtcccccaggacctgg
agcccctgaccatcttgtactatgtgggcagaacccccaaggtggagcag
ctgtccaacatggtggtgaagtcgtgtaagtgcagctga (SEQ ID NO : 13)

The engineered vectors described herein comprise at least one TGF-β polypeptide. In some embodiments of any of the aspects described herein, the TGF-β polypeptide is a TGF-β1, TGF-β2, or a TGF-β3 polypeptide. Engineered vector compositions are described in further detail below.

### Gene Therapy Vectors

In one aspect, described herein are engineered vectors for delivering TGF-β polypeptides to neuronal and/or retinal tissues. As discussed herein above, non-limiting examples include vectors for delivering TGF-β polypeptides to e.g., retinal tissue, including, for example, vectors for expressing TGF-β polypeptides in retinal cone cells. Gene therapy vectors useful in the methods and compositions described herein will generally include a nucleic acid sequence encoding a TGF-β polypeptide, operably linked to regulatory sequences sufficient to drive expression of the TGF-β polypeptide transgene in the target tissue. Thus, as a non-limiting example, a gene therapy vector as described herein can include a retina-specific promoter operably linked to a nucleic acid sequence encoding a transforming growth factor beta (TGF-β) polypeptide.

A vector can encompass any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell.

In some embodiments of any of the aspects, the vector is a viral vector. In some embodiments of any of the aspects, the engineered vector is selected from the group consisting of: an adeno-associated virus (AAV) vector; an adenovirus vector; and a lentiviral vector.

AAV vectors can include but are not limited to, AAV serotype 1, AAV serotype 2, AAV serotype 3 (including types 3A and 3B), AAV serotype 4, AAV serotype 5, AAV serotype 6, AAV serotype 7, AAV serotype 8, AAV serotype 9, AAV serotype 10, AAV serotype 11, avian AAV, bovine AAV, canine AAV, equine AAV, ovine AAV and Clade F AAV. See, e.g., Bernard N. Fields et al., Virology, volume 2, chapter 69 (4th ed., Lippincott-Raven Publishers). See also Gao et al. (2004) J. Virology 78: 6381-6388 regarding the identification of AAV serotypes and clades, as well as Table 1).

The genomic sequences of various serotypes of AAV, as well as the sequences of the native terminal repeats (TRs), Rep proteins, and capsid subunits are known in the art. Exemplary but non-limiting examples of such sequences may be found in the literature or in public databases such as GenBank® Database. See, e.g., GenBank® Database Accession Numbers NC_002077.1, NC_001401.2, NC_001729.1, NC_001863.1, NC_001829.1, NC_006152.1, NC_001862.1, AF513851.1, AF513852.1, the disclosures of which are incorporated by reference herein for teaching parvovirus and AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao etal. (2002) Proc. Nat. Acad. Sci. USA 99:11854; international patent publications WO 00/28061, WO 99/6160 and WO 98/11244; and U.S. Patent No. 6,156,303; the disclosures of which are incorporated by reference herein for teaching parvovirus and AAV nucleic acid and amino acid sequences.

The capsid structures of viral vectors, e.g., AAVs, are described in more detail in BERNARD N. FIELDS et al., Virology, Volume 2, Chapters 69 & 70 (4th ed., Lippincott-Raven Publishers). See also, description of the crystal structure of AAV2 (Xie et al. (2002) Proc. Nat. Acad. Sci. 99: 10405-10), AAV4 (Padron et al. (2005) J. Virol. 79: 5047-58), AAV5 (Walters et al. (2004) J. Virol. 78: 3361-71) and CPV (Xie et al. (1996) J. Mol. Biol. 6:497-520 and Tsao et al. (1991) Science 251: 1456-64).

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997), the contents of which are incorporated herein. Further, production of AAV vectors is further described, e.g., in U.S. Patent Number 9,441,206, the contents of which are incorporated herein by reference in their entirety.

Viral vectors produced in a viral expression system can be released (i.e. set free from the cell that produced the vector) using any standard technique. For example, viral vectors can be released via mechanical methods, for example microfluidization, centrifugation, or sonication, or chemical methods, for example lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). Viral vectors can be released from the cell, for example, via sonication and recovered via purification of clarified lysate using column chromatography.

Viral vectors can comprise the genome, in part or entirety, of any naturally occurring and/or recombinant viral vector nucleotide sequence (e.g., AAV, adenovirus, lentivirus, etc.) or variant. Viral vector variants can have genomic sequences of significant homology at the nucleic acid and amino acid levels, produce viral vectors which are generally physical and functional equivalents, replicate by similar mechanisms, and assemble by similar mechanisms.

Variant viral vector sequences can be used to produce viral vectors in the viral expression systems described herein. For example, a variant vector can have a sequence having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, adenovirus, lentivirus, etc.).

It is to be understood that a viral expression system can be modified to include any necessary elements required to complement a given viral vector during its production using methods described herein. For example, in certain embodiments, a nucleic acid cassette is flanked by terminal repeat sequences. In one embodiment, for the production of recombinant (rAAV) vectors, the AAV expression system will further comprise at least one of a recombinant AAV plasmid, a plasmid expressing Rep, a plasmid expressing Cap, and an adenovirus helper plasmid. Complementary elements for a given viral vector are well known in the art, and a skilled practitioner would be capable of modifying viral expression systems as described herein accordingly.

A viral expression system for manufacturing an AAV vector (e.g., an AAV expression system) can comprise Replication (Rep) genes and/or Capsid (Cap) genes in trans, for example, under the control of an inducible promoter. Expression of Rep and Cap can be under the control of one inducible promoter, such that expression of these genes is turned “on” together, or under control of two separate inducible promoters that are turned “on” by distinct inducers. On the left side of the AAV genome there are two promoters called p5 and p19, from which two overlapping messenger ribonucleic acids (mRNAs) of different length can be produced. Each of these contains an intron which can either be spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Numbers US5478745A; US5622856A; US5658776A; US6440742B1; US6632670B1; US6156303A; US8007780B2; US6521225B1; US7629322B2; US6943019B2; US5872005A; and U.S. Pat. Application Numbers US 2017/0130245; US20050266567A1; US20050287122A1; the contents of each of these are incorporated herein by reference in their entireties.

In some embodiments of any of the aspects, the vector described herein further comprises a 5′ Inverted Terminal Repeat (ITR) sequence. In some embodiments the 5′ ITR polynucleotide sequence comprises

ctgcgcgctcgctcgctcactgaggccgcccgggcaaagcccgggcgtcg
ggcgacctttggtcgcccggcctcagtgagcgagcgagcgcgcagagagg
gagtggccaactccatcactaggggttcct (SEQID NO: 14)

In some embodiments of any of the aspects, the vector described herein further comprises a 3′ ITR sequence. In some embodiments the 3′ ITR polynucleotide sequence comprises

aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcag (SEQ ID NO:15).

Any of a variety of different host cell systems can be used to produce viral, e.g., AAV, vectors for use in the methods and compositions described herein. Some methods involve the co-transfection of two or three plasmids containing AAV genes, adenovirus helper genes, and a vector genome. Mammalian host cells can be used, such as HEK293 cells. Alternatively, insect cells have also been used, taking advantage of a baculovirus expression system to drive high efficiency expression of the necessary components for AAV viral particle assembly. See, e.g., Urabe et al., Hum. Gene Ther. 13: 1935-1943 (2002), the contents of which are incorporated herein by reference. Exemplary insect cells include but are not limited to Sf9, Sf21, Hi-5, and S2 insect cell lines. The baculovirus expression system is designed for efficient large-scale viral production and expression of recombinant proteins from baculovirus-infected insect cells. Baculovirus expression systems are further described in, e.g., U.S. Patent Numbers US6919085B2; US6225060B1; US5194376A; the contents of each are incorporated herein by reference in their entireties.

In another embodiment, the viral production system can be a cell-free system. Cell-free systems for viral vector production are described in, for example, Cerqueira A., et al. J. Virol. 90: 1096-1107 (2016); Sheng J., et al. The Royal Society of Chemistry, 2017; and Svitkin Y.V., and Sonenberg N., J. Virol. 77: 6551-6555 ( 2003), the contents of each of which are incorporated herein by reference in their entireties.

Conditions sufficient for the replication and packaging of the AAV particles can be, e.g., the presence of AAV sequences sufficient for replication of an AAV template and encapsidation into AAV capsids (e.g., AAV rep sequences and AAV cap sequences) and helper sequences from adenovirus and/or herpesvirus. In particular embodiments, the AAV template comprises two AAV ITR sequences, which are located 5′ and 3′ to the heterologous payload nucleic acid sequence, although they need not be directly contiguous thereto.

In some embodiments, the AAV template comprises an ITR that is not resolved by Rep to make duplexed AAV vectors as described in international patent publication WO 2001/092551.

The AAV template and AAV rep and/or cap sequences are provided under conditions such that viral vector comprising AAV template packaged within an AAV capsid is produced in the cell. The preparation method can further comprise the step of collecting the viral vector from the culture. In one approach, the viral vector can be collected by lysing the cells, e.g., after removing the cells from the culture medium, e.g., by centrifugation. In another embodiment, the viral vector can be collected from the culture medium, e.g., to isolate vector particles that are secreted from the cells. Some or all of the medium can be removed from the culture one time or more than one time, e.g., at regular intervals during the culture for collection of rAAV (such as every 12, 18, 24, or 36 hours, or longer extended time that is compatible with cell viability and vector production), e.g., beginning about 48 hours post-transfection. After removal of the medium, fresh medium, with or without additional nutrient supplements, can be added to the culture. In one approach, the cells can be cultured in a perfusion system such that medium constantly flows over the cells and is collected for isolation of secreted rAAV. Collection of rAAV from the medium can continue for as long as the transfected cells remain viable, e.g., 48, 72, 96, or 120 hours or longer post-transfection, or in the case of the use of an inducible promoter system to express the components necessary for vector assembly, e.g., 48, 72, 96, or 120 hours or longer post-induction. In certain embodiments, the collection of secreted rAAV is carried out with serotypes of AAV (such as AAV8 and AAV9), which do not bind or only loosely bind to the producer cells. In other embodiments, the collection of secreted rAAV is carried out with heparin binding serotypes of AAV (e.g., AAV2) that have been modified so as to not bind to the cells in which they are produced. Examples of suitable modifications, as well as rAAV collection techniques, are disclosed in U.S. Pat. Application publication No. 2009/0275107, which is incorporated by reference herein in its entirety.

In the event that a producer cell line does not stably or transiently express rep or cap, these sequences are to be provided to the AAV expression system. AAV rep and cap sequences can be provided by any method known in the art. Current protocols typically express the AAV rep/cap genes on a single plasmid. The AAV replication and packaging sequences need not be provided together, although it may be convenient to do so. The AAV rep and/or cap sequences can be provided by any viral or non-viral vector. For example, the rep/cap sequences can be provided by a hybrid adenovirus or herpesvirus vector (e.g., inserted into the Ela or E3 regions of a deleted adenovirus vector). EBV vectors can also be employed to express the AAV cap and rep genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV rep/ cap sequences will not be flanked by the TRs, to prevent rescue and/or packaging of these sequences.

The AAV template can be provided to the cell using any method known in the art. For example, the template can be supplied by a non-viral (e.g., plasmid) or viral vector. In particular embodiments, the AAV template is supplied by a herpesvirus or adenovirus vector (e.g., inserted into the Ela or E3 regions of a deleted adenovirus). As another illustration, Palombo et al., J. Virol. 72:5025 (1998), describes a baculovirus vector carrying a reporter gene flanked by the AAV TRs. EBV vectors may also be employed to deliver the template, as described above with respect to the rep/cap genes.

In another representative embodiment, the AAV template is provided by a replicating rAAV virus. In still other embodiments, an AAV provirus comprising the AAV template is stably integrated into the chromosome of the producer cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., Nature Med. 3:1295 (1997), and U.S. Pat. Nos. 6,040,183 and 6,093,570, which are incorporated herein by reference.

Further, the helper virus functions can be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV cap and rep sequences and the helper virus sequences (e.g., adenovirus sequences) on a single helper construct. In one embodiment, expression of at least one gene product encoded by the single helper construct is controlled by an inducible promoter. This helper construct can be a non-viral or viral construct. As one non-limiting illustration, the helper construct can be a hybrid adenovirus or hybrid herpesvirus comprising the AAV rep and/or cap genes.

In some embodiments, the AAV rep and/or cap sequences and the adenovirus helper sequences are supplied by a single adenovirus helper vector. This vector can further comprise the AAV template. The AAV rep and/or cap sequences and/or the AAV template can be inserted into a deleted region (e.g., the E1 a or E3 regions) of the adenovirus. In one embodiment, expression of at least one gene product encoded by the AAV template is controlled by an inducible promoter.

In a further embodiment, the AAV rep and/or cap sequences and the adenovirus helper sequences are supplied by a single adenovirus helper vector. According to this embodiment, the AAV template can be provided as a plasmid template.

In another illustrative embodiment, the AAV rep and/or cap sequences and adenovirus helper sequences are provided by a single adenovirus helper vector, and the AAV template is integrated into the cell as a provirus. Alternatively, the AAV template is provided by an EBV vector that is maintained within the cell as an extrachromosomal element (e.g., as an EBV based nuclear episome).

Use of inducible and repressible promoters as described herein can be used to achieve temporal regulation of any of the toxic proteins required for viral vector production, for example, rep and cap. In one embodiment, inducible and/or repressible promoters provide for careful fine tuning of expression of a toxic protein, such that one can tailor the start and stop of the expression to achieve the desired level of expression, and at the desired timing during production.

In a further exemplary embodiment, the AAV rep and/or cap sequences and adenovirus helper sequences are provided by a single adenovirus helper. The AAV template can be provided as a separate replicating viral vector. For example, the AAV template can be provided by an AAV particle or a second recombinant adenovirus particle.

According to the foregoing methods, a hybrid adenovirus vector typically comprises the adenovirus 5′ and 3′ cis sequences sufficient for adenovirus replication and packaging (i.e., the adenovirus terminal repeats and PAC sequence). The AAV rep and/or cap sequences and, if present, the AAV template are embedded in the adenovirus backbone and are flanked by the 5′ and 3′ cis sequences, so that these sequences may be packaged into adenovirus capsids. As described above, the adenovirus helper sequences and the AAV rep and/or cap sequences are generally not flanked by TRs so that these sequences are not packaged into the AAV virions. Zhang et al., Gene Ther. 18:704 ((2001)) describe a chimeric helper comprising both adenovirus and the AAV rep and/or cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., Gene Ther. 6:986 (1999) and WO 00/17377).

AAV vector stocks free of contaminating helper virus can be obtained by any method known in the art. For example, AAV and helper virus can be readily differentiated based on size. AAV can also be separated away from helper virus based on affinity for a heparin substrate (Zolotukhin et al. Gene Ther. 6:973 (1999)). Deleted replication-defective helper viruses can be used so that any contaminating helper virus is not replication competent. As a further alternative, an adenovirus helper lacking late gene expression may be employed, as only adenovirus early gene expression is required to mediate packaging of AAV. Adenovirus mutants which are defective for late gene expression are known in the art (e.g., ts100K and ts149 adenovirus mutants).

The methods described herein are suitable for production of all serotypes and chimeras of AAV, e.g., AAV1, AAV2, AAV3, AAV4, AAV5, AAV6, AAV7, AAV8, AAV9, AAV10, AAV11, AAV12, AAV13, and any chimeras thereof.

In certain embodiments, the production of viral vectors provides at least about 1 × 10⁴ vector genome-containing particles per cell prior to purification, e.g., at least about 2 × 10⁴, 3 × 10⁴, 4 × 10⁴, 5 × 10⁴, 6 × 10⁴, 7 × 10⁴, 8 × 10⁴, 9 × 10⁴, or 1 × 10⁵ or more vector genome-containing particles per cell prior to purification. In other embodiments, the method provides at least about 1 × 10¹² purified vector genome-containing particles per liter of cell culture, e.g., at least about 5 × 10¹², 1 × 10¹³, 5 × 10¹³, or 1 × 1 0¹⁴ or more purified vector genome-containing particles per liter of cell culture.

Exemplary AAV genomes that can be used for the engineered vector compositions described herein are provided in Tables 1-3.

TABLE 1

AAV Genomes

| Complete Genomes | GenBank® Accession Number | | GenBank® Accession Number | | GenBank® Accession Number |
|---|---|---|---|---|---|
| | | Hu T88 | AY695375 | Clade E | |
| AAV1 | NC_002077,AF06 3497 | Hu T71 | AY695374 | Rh38 | AY530558 |
| AAV2 | NC_001401 | Hu T70 | AY695373 | Hu66 | AY530626 |
| AAV 3 | NC_001729 | Hu T40 | AY695372 | Hu42 | AY530605 |
| AAV3B | NC_001863 | Hu T32 | AY695371 | Hu67 | AY530627 |
| AAV4 | NC_001829 | Hu T17 | AY695370 | Hu40 | AY530603 |
| AAV5 | Y18065, AF085716 | Hu LG15 | AY695377 | Hu41 | AY530604 |
| AAV6 | NC_001862 | | | Hu37 | AY530600 |
| AAV | AY186198, AY629583, NC_004828 | Clade C | | Rh40 | AY530559 |
| Avian AAV strain DA-1 | NC_006263, AY629583 | Hu9 | AY530629 | Rh2 | AY243 007 |
| Bovine AAV | NC_005889, AY388617 | Hu10 | AY530576 | Bb1 | AY243 023 |
| | | Hu11 | AY530577 | Bb2 | AY243 022 |
| Clade A | | Hu53 | AY530615 | Rh10 | AY243015 |
| AAV1 | NC_002077,AF06 3497 | Hu55 | AY530617 | Hu17 | AY530582 |
| AAV6 | NC_001862 | Hu54 | AY530616 | Hu6 | AY530621 |
| Hu.48 | AY530611 | Hu7 | AY530628 | Rh25 | AY530557 |
| Hu 43 | AY530606 | Hu18 | AY530583 | Pi2 | AY530554 |
| Hu 44 | AY530607 | Hu15 | AY530580 | Pi1 | AY530553 |
| Hu 46 | AY530609 | Hu16 | AY530581 | Pi3 | AY530555 |
| | | Hu25 | AY530591 | Rh57 | AY530569 |
| Clade B | | Hu60 | AY530622 | Rh50 | AY530563 |
| Hu. 19 | AY530584 | Ch5 | AY243021 | Rh49 | AY530562 |
| Hu. 20 | AY530586 | Hu3 | AY530595 | Hu39 | AY530601 |
| Hu 23 | AY530589 | Hu1 | AY530575 | Rh58 | AY530570 |
| Hu22 | AY530588 | Hu4 | AY530602 | Rh61 | AY530572 |
| Hu24 | AY530590 | Hu2 | AY530585 | Rh52 | AY530565 |
| Hu21 | AY530587 | Hu61 | AY530623 | Rh53 | AY530566 |
| Hu27 | AY530592 | | | Rh51 | AY530564 |
| Hu28 | AY530593 | Clade D | | Rh64 | AY530574 |
| Hu 29 | AY530594 | Rh62 | AY530573 | Rh43 | AY530560 |
| Hu63 | AY530624 | Rh48 | AY530561 | AAV8 | AF513852 |
| Hu64 | AY530625 | Rh54 | AY530567 | Rh8 | AY242997 |
| Hu13 | AY530578 | Rh55 | AY530568 | Rh1 | AY530556 |
| Hu56 | AY530618 | Cy2 | AY243020 | | |
| Hu57 | AY530619 | AAV7 | AF513851 | Clade F | |
| Hu49 | AY530612 | Rh35 | AY243000 | Hu14 (AAV9) | AY530579 |
| Hu58 | AY530620 | Rh37 | AY242998 | Hu31 | AY530596 |
| Hu34 | AY530598 | Rh36 | AY242999 | Hu32 | AY530597 |
| Hu35 | AY530599 | Cy6 | AY243016 | | |
| AAV2 | NC_001401 | Cy4 | AY243018 | Clonal Isolate | |
| Hu45 | AY530608 | Cy3 | AY243019 | AAV5 | Y18065, AF085716 |
| Hu47 | AY530610 | Cy5 | AY243017 | AAV 3 | NC_001729 |
| Hu51 | AY530613 | Rh13 | AY243013 | AAV 3B | NC_001863 |
| Hu52 | AY530614 | | | AAV4 | NC_001829 |
| Hu T41 | AY695378 | | | Rh34 | AY243001 |
| Hu S17 | AY695376 | | | Rh33 | AY243002 |
| | | | | Rh32 | AY243003 |

TABLE 2

Exemplary AAV Genome and Capsid Accession Nos.

| | | |
|---|---|---|
| Virus and Serotype | Genome Accession No. | Capsid/VP1 Accession No. |
| AAV1 | NC_002077.1 | NP_049542.1 |
| AAV2 | NC_001401.2 | YP_680426.1 |
| AAV3A | NC_001729.1 | NP_043941.1 |
| AAV3B | NC_001863.1 | NP_045760.1 |
| AAV4 | NC_001829.1 | NP_044927.1 |
| AAV5 | NC_006152.1 | YP_068409.1 |
| AAV6 | NC_001862.1 | NP_045758.1 |
| AAV7 | AF513851.1 | AAN03855.1 |
| AAV8 | AF513852.1 | AAN03857.1 |
| AAV9 | AY530579.1 | AAS99264.1 |
| AAV10 | AY631965.1* | AAT46337.1 |
| AAV11 | AY631966.1* | AAT46339.1 |
| AAV13 | EU285562.1 | ABZ10812.1 |

* Incomplete sequence

Table 3 describes exemplary AAV serotypes and exemplary published corresponding capsid sequences that can be used as the AAV capsid in the rAAV vector described herein, or with any combination with wild type capsid proteins and/or other chimeric or variant capsid proteins and each is incorporated herein.

TABLE 3

AAV Serotypes and exemplary published corresponding capsid sequence The sequences noted in this table are known in the art and are incorporated herein by reference in their entireties

| | |
|---|---|
| Serotype and where capsid sequence is published | Serotype and where capsid sequence is published |
| AAV3.3b See US20030138772 SEQ ID NO: 72 | AAV3-3 See US20150315612 SEQ ID NO: 200 |
| AAV3-3 See US20150315612 SEQ ID NO: 217 | AAV3a See US6156303 SEQ ID NO: 5 |
| AAV3a See US6156303 SEQ ID NO: 9 | AAV3b See US6156303 SEQ ID NO: 6 |
| AAV3b See US6156303 SEQ ID NO: 10 | AAV3b See US6156303 SEQ ID NO: 1 |
| AAV4 See US20140348794 SEQ ID NO: 17 | AAV4 See US20140348794 SEQ ID NO: 5 |
| AAV4 See US20140348794 SEQ ID NO: 3 | AAV4 See US20140348794 SEQ ID NO: 14 |
| AAV4 See US20140348794 SEQ ID NO: 15 | AAV4 See US20140348794 SEQ ID NO: 19 |
| AAV4 See US20140348794 SEQ ID NO: 12 | AAV4 See US20140348794 SEQ ID NO: 13 |
| AAV4 See US20140348794 SEQ ID NO: 7 | AAV4 See US20140348794 SEQ ID NO: 8 |
| AAV4 See US20140348794 SEQ ID NO: 9 | AAV4 See US20140348794 SEQ ID NO: 2 |
| AAV4 See US20140348794 SEQ ID NO: 10 | AAV4 See US20140348794 SEQ ID NO: 11 |
| AAV4 See US20140348794 SEQ ID NO: 18 | AAV4 See US20030138772 SEQ ID NO: 63, US20160017295 SEQ ID NO: See US20140348794 SEQ ID NO: 4 |
| | AAV4 See US20140348794 SEQ ID NO: 16 |
| AAV4 See US20140348794 SEQ ID NO: 20 | AAV4 See US20140348794 SEQ ID NO: 6 |
| AAV4 See US20140348794 SEQ ID NO: 1 | AAV42.2 See US20030138772 SEQ ID NO: 9 |
| AAV42.2 See US20030138772 SEQ ID NO: 102 | AAV42.3b See US20030138772 SEQ ID NO: 36 |
| AAV42.3B See US20030138772 SEQ ID NO: 107 | AAV42.4 See US20030138772 SEQ ID NO: 33 |
| AAV42.4 See US20030138772 SEQ ID NO: 88 | AAV42.8 See US20030138772 SEQ ID NO: 27 |
| AAV42.8 See US20030138772 SEQ ID NO: 85 | AAV43.1 See US20030138772 SEQ ID NO: 39 |
| AAV43.1 See US20030138772 SEQ ID NO: 92 | AAV43.12 See US20030138772 SEQ ID NO: 41 |
| AAV43.12 See US20030138772 SEQ ID NO: 93 | AAV8 See US20150159173 SEQ ID NO: 15 |
| AAV8 See US20150376240 SEQ ID NO: 7 | AAV8 See US20030138772 SEQ ID NO: 4, US20150315612 SEQ ID NO: 182 |
| | AAV8 See US20030138772 SEQ ID NO: 95, US20140359799 SEQ ID NO: 1, US20150159173 SEQ ID NO: 31, |
| | US20160017295 SEQ ID NO: 8, US7198951 SEQ ID NO: 7, US20150315612 SEQ ID NO: 223 |
| AAV8 See US20150376240 SEQ ID NO: 8 | AAV8 See US20150315612 SEQ ID NO: 214 |
| AAV-8b See US20150376240 SEQ ID NO: 5 | AAV-8b See US20150376240 SEQ ID NO: 3 |
| AAV-8h See US20150376240 SEQ ID NO: 6 | AAV-8h See US20150376240 SEQ ID NO: 4 |
| AAV9 See US20030138772 SEQ ID NO: 5 | AAV9 See US7198951 SEQ ID NO: 1 |
| AAV9 See US20160017295 SEQ ID NO: 9 | AAV9 See US20030138772 SEQ ID NO: 100, US7198951 SEQ ID NO: 2 |
| | AAV9 See US7198951 SEQ ID NO: 3 |
| AAV9 (AAVhu.14) See US20150315612 SEQ ID NO: 3 | AAV9 (AAVhu.14) See US20150315612 SEQ ID NO: 123 |
| AAVA3.1 See US20030138772 SEQ ID NO: 120 | AAVA3.3 See US20030138772 SEQ ID NO: 57 |
| AAVA3.3 See US20030138772 SEQ ID NO: 66 | AAVA3.4 See US20030138772 SEQ ID NO: 54 |
| AAVA3.4 See US20030138772 SEQ ID NO: 68 | AAVA3.5 See US20030138772 SEQ ID NO: 55 |
| AAVA3.5 See US20030138772 SEQ ID NO: 69 | AAVA3.7 See US20030138772 SEQ ID NO: 56 |
| AAVA3.7 See US20030138772 SEQ ID NO: 67 | AAV29. See (AAVbb. 1) 161 US20030138772 SEQ ID NO: 11 |
| AAVC2 See US20030138772 SEQ ID NO: 61 | AAVCh.5 See US20150159173 SEQ ID NO: 46, US20150315612 SEQ ID NO: 234 |
| | AAVcy.2 (AAV13.3) See US20030138772 SEQ ID NO: 15 |
| AAV24.1 See US20030138772 SEQ ID NO: 101 | AAVcy.3 (AAV24.1) See US20030138772 SEQ ID NO: 16 |
| AAV27.3 See US20030138772 SEQ ID NO: 104 | AAVcy.4 (AAV27.3) See US20030138772 SEQ ID NO: 17 |
| AAVcy.5 See US20150315612 SEQ ID NO: 227 | AAV7.2 See US20030138772 SEQ ID NO: 103 |
| AAVcy.5 (AAV7.2) See US20030138772 SEQ ID NO: 18 | AAV16.3 See US20030138772 SEQ ID NO: 105 |
| AAVcy.6 (AAV16.3) See US20030138772 SEQ ID NO: 10 | AAVcy.5 See US20150159173 SEQ ID NO: 8 |
| AAVcy.5 See US20150159173 SEQ ID NO: 24 | AAVCy.5R1 See US20150159173 |
| AAVCy.5R2 See US20150159173 | AAVCy.5R3 See y |
| AAVCy.5R4 See US20150159173 | AAVDJ See US20140359799 SEQ ID NO: 3, US7588772 SEQ ID NO: 2 |
| | AAVDJ See US20140359799 SEQ ID NO: 2, US7588772 SEQ ID NO: 1 |
| | AAVDJ-8 See US7588772; Grimm et al 2008 |
| AAVDJ-8 See US7588772; Grimm et al 2008 | AAVF5 See US20030138772 SEQ ID NO: 110 |
| AAVH2 See US20030138772 SEQ ID NO: 26 | AAVH6 See US20030138772 SEQ ID NO: 25 |
| AAVhEl.1 See US9233131 SEQ ID NO: 44 | AAVhErl.14 See US9233131 SEQ ID NO: 46 |
| AAVhErl.16 See US9233131 SEQ ID NO: 48 | AAVhErl.18 See US9233131 SEQ ID NO: 49 |
| AAVhErl.23 (AAVhEr2.29) See US9233131 SEQ ID NO: 53 | AAVhErl.35 See US9233131 SEQ ID NO: 50 |
| AAVhErl.36 See US9233131 SEQ ID NO: 52 | AAVhErl.5 See US9233131 SEQ ID NO: 45 |
| AAVhErl.7 See US9233131 SEQ ID NO: 51 | AAVhErl.8 See US9233131 SEQ ID NO: 47 |
| AAVhEr2.16 See US9233131 SEQ ID NO: 55 | AAVhEr2.30 See US9233131 SEQ ID NO: 56 |
| AAVhEr2.31 See US9233131 SEQ ID NO: 58 | AAVhEr2.36 See US9233131 SEQ ID NO: 57 |
| AAVhEr2.4 See US9233131 SEQ ID NO: 54 | AAVhEr3.1 See US9233131 SEQ ID NO: 59 |
| AAVhu.l See US20150315612 SEQ ID NO: 46 | AAVhu.l See US20150315612 SEQ ID NO: 144 |
| AAVhu.lO (AAV16.8) See US20150315612 SEQ ID NO: 56 | AAVhu.lO (AAV16.8) See US20150315612 SEQ ID NO: 156 |
| AAVhu.l l (AAV16.12) See US20150315612 SEQ ID NO: 57 | AAVhu.l l (AAV16.12) See US20150315612 SEQ ID NO: 153 |
| AAVhu.12 See US20150315612 SEQ ID NO: 59 | AAVhu.12 See US20150315612 SEQ ID NO: 154 |
| AAVhu.13 See US20150159173 SEQ ID NO: 16, US20150315612 SEQ ID NO: 71 | |
| AAVhu.13 See US20150159173 SEQ ID NO: 32, US20150315612 SEQ ID NO: 129 | |
| AAVhu.136.1 See US20150315612 SEQ ID NO 165 | AAVhu.140.1 See US20150315612 SEQ ID NO 166 |
| AAVhu.140.2 See US20150315612 SEQ ID NO 167 | AAVhu.145.6 See y SEQ ID No: 178 |
| AAVhu.15 See US20150315612 SEQ ID NO: 147 | AAVhu.15 (AAV33.4) See US20150315612 SEQ ID NO: 50 |
| AAVhu.156.1 See US20150315612 SEQ ID No: 179 | AAVhu.16 See US20150315612 SEQ ID NO 148 |
| AAVhu.16 (AAV33.8) See US20150315612 SEQ ID NO 51 | AAVhu.17 See US20150315612 SEQ ID NO 83 |
| AAVhu.17 (AAV33.12) See US20150315612 SEQ ID NO 4 | AAVhu.172.1 See US20150315612 SEQ ID NO 171 |
| AAVhu.172.2 See US20150315612 SEQ ID NO 172 | AAVhu.173.4 See US20150315612 SEQ ID NO 173 |
| AAVhu.173.8 See US20150315612 SEQ ID NO 175 | AAVhu.18 See US20150315612 SEQ ID NO 52 |
| AAVhu.18 See US20150315612 SEQ ID NO 149 | AAVhu.19 See US20150315612 SEQ ID NO 62 |
| AAVhu.19 See US20150315612 SEQ ID NO 133 | AAVhu.2 See US20150315612 SEQ ID NO 48 |
| AAVhu.2 See US20150315612 SEQ ID NO 143 | AAVhu.20 See US20150315612 SEQ ID NO 63 |
| AAVhu.20 See US20150315612 SEQ ID NO 134 | AAVhu.21 See US20150315612 SEQ ID NO 65 |
| AAVhu.21 See US20150315612 SEQ ID NO 135 | AAVhu.22 See US20150315612 SEQ ID NO 67 |
| AAVhu.22 239 US20150315612 SEQ ID NO 138 | AAVhu.23 See US20150315612 SEQ ID NO 60 |
| AAVhu.23.2 See US20150315612 SEQ ID NO 137 | AAVhu.24 See US20150315612 SEQ ID NO 66 |
| AAVhu.24 See US20150315612 SEQ ID NO 136 | AAVhu.25 See US20150315612 SEQ ID NO 49 |
| AAVhu.25 See US20150315612 SEQ ID NO 146 | AAVhu.26 See US20150159173 SEQ ID NO 17, US20150315612 SEQ ID NO: 61 |
| | AAVhu.26 See US20150159173 SEQ ID NO: 33, US20150315612 SEQ |
| | AAVhu.27 See US20150315612 SEQ ID NO: 64 |
| AAVhu.27 See US20150315612 SEQ ID NO: 140 | AAVhu.28 See US20150315612 SEQ ID NO: 68 |
| AAVhu.28 See US20150315612 SEQ ID NO: 130 | AAVhu.29 See US20150315612 SEQ ID NO: 69 |
| AAVhu.29 See US20150159173 SEQ ID NO: 42, US20150315612 SEQ ID NO: 132 | |
| AAVhu.29 See US20150315612 SEQ ID NO: 225 | AAVhu.29R See US20150159173 |
| AAVhu.3 See US20150315612 SEQ ID NO: 44 | AAVhu.3 See US20150315612 SEQ ID NO: 145 |
| AAVhu.30 See US20150315612 SEQ ID NO: 70 | AAVhu.30 See US20150315612 SEQ ID NO: 131 |
| AAVhu.31 See US20150315612 SEQ ID NO: 1 | AAVhu.31 See US20150315612 SEQ ID NO: 121 |
| AAVhu.32 See US20150315612 SEQ ID NO: 2 | AAVhu.32 See US20150315612 SEQ ID NO: 122 |
| AAVhu.33 See US20150315612 SEQ ID NO: 75 | AAVhu.33 See US20150315612 SEQ ID NO: 124 |
| AAVhu.34 See US20150315612 SEQ ID NO: 72 | AAVhu.34 See US20150315612 SEQ ID NO: 125 |
| AAVhu.35 See US20150315612 SEQ ID NO: 73 | AAVhu.35 See US20150315612 SEQ ID NO: 164 |
| AAVhu.36 See US20150315612 SEQ ID NO: 74 | AAVhu.36 See US20150315612 SEQ ID NO: 126 |
| AAVhu.37 See US20150159173 SEQ ID NO: 34, US20150315612 SEQ ID NO: 88 | |
| AAVhu.37 (AAV106.1) See US20150315612 SEQ ID NO: 10, US20150159173 SEQ ID NO: 18 | |
| AAVhu.38 See US20150315612 SEQ ID NO 161 | AAVhu.39 See US20150315612 SEQ ID NO 102 |
| AAVhu.39 (AAVLG-9) See US20150315612 SEQ ID NO 24 | AAVhu.4 See US20150315612 SEQ ID NO 47 |
| AAVhu.4 See US20150315612 SEQ ID NO 141 | AAVhu.40 See US20150315612 SEQ ID NO 87 |
| AAVhu.40 (AAV114.3) See US20150315612 SEQ ID No: 11 | AAVhu.41 See US20150315612 SEQ ID NO: 91 |
| AAVhu.41 (AAV127.2) See US20150315612 SEQ ID NO: 6 | AAVhu.42 See US20150315612 SEQ ID NO: 85 |
| AAVhu.42 (AAV127.5) See US20150315612 SEQ ID NO: 8 | AAVhu.43 See US20150315612 SEQ ID NO: 160 |
| AAVhu.43 See US20150315612 SEQ ID NO: 236 | AAVhu.43 (AAV128.1) See US20150315612 SEQ ID NO: 80 |
| AAVhu.44 See US20150159173 SEQ ID NO: 45, US20150315612 SEQ ID NO: 158 | |
| AAVhu.44 (AAV128.3) See US20150315612 SEQ ID NO: 81 | AAVhu.44Rl See US20150159173 |
| AAVhu.44R2 See US20150159173 | AAVhu.44R3 See US20150159173 |
| AAVhu.45 See US20150315612 SEQ ID NO: 76 | AAVhu.45 See US20150315612 SEQ ID NO: 127 |
| AAVhu.46 See US20150315612 SEQ ID NO: 82 | AAVhu.46 See US20150315612 SEQ ID NO: 159 |
| AAVhu.46 See US20150315612 SEQ ID NO: 224 | AAVhu.47 See US20150315612 SEQ ID NO: 77 |
| AAVhu.47 See US20150315612 SEQ ID NO: 128 | AAVhu.48 See US20150159173 SEQ ID NO: 38 |
| AAVhu.48 See US20150315612 SEQ ID NO: 157 | AAVhu.48 (AAV130.4) See US20150315612 SEQ ID NO: 78 |
| AAVhu.48Rl See US20150159173 | AAVhu.48R2 See US20150159173 |
| AAVhu.48R3 See US20150159173 | AAVhu.49 See US20150315612 SEQ ID NO 209 |
| AAVhu.49 See US20150315612 SEQ ID NO 189 | AAVhu.5 See US20150315612 SEQ ID NO 45 |
| AAVhu.5 See US20150315612 SEQ ID NO 142 | AAVhu.51 See US20150315612 SEQ ID NO 208 |
| AAVhu.51 See US20150315612 SEQ ID NO 190 | AAVhu.52 See US20150315612 SEQ ID NO 210 |
| AAVhu.52 See US20150315612 SEQ ID NO 191 | AAVhu.53 See US20150159173 SEQ ID NO 19 |
| AAVhu.53 See US20150159173 SEQ ID NO 35 | AAVhu.53 (AAV145.1) See US20150315612 SEQ ID NO 176 |
| AAVhu.54 See US20150315612 SEQ ID NO 188 | AAVhu.54 (AAV145.5) See US20150315612 SEQ ID No: 177 |
| AAVhu.55 See US20150315612 SEQ ID NO 187 | AAVhu.56 See US20150315612 SEQ ID NO 205 |
| AAVhu.56 (AAV145.6) See US20150315612 SEQ ID NO 168 | AAVhu.56 (AAV145.6) See US20150315612 SEQ ID NO 192 |
| AAVhu.57 See US20150315612 SEQ ID NO 206 | AAVhu.57 See US20150315612 SEQ ID NO 169 |
| AAVhu.57 See US20150315612 SEQ ID NO 193 | AAVhu.58 See US20150315612 SEQ ID NO 207 |
| AAVhu.58 See US20150315612 SEQ ID NO 194 | AAVhu.6 (AAV3.1) See US20150315612 SEQ ID NO: 5 |
| AAVhu.6 (AAV3.1) See US20150315612 SEQ ID NO: 84 | AAVhu.60 See US20150315612 SEQ ID NO: 184 |
| AAVhu.60 (AAV161.10) See US20150315612 SEQ ID NO: 170 | AAVhu.61 See US20150315612 SEQ ID NO: 185 |
| AAVhu.61 (AAV161.6) See US20150315612 SEQ ID NO: 174 | AAVhu.63 See US20150315612 SEQ ID NO: 204 |
| AAVhu.63 See US20150315612 SEQ ID NO: 195 | AAVhu.64 See US20150315612 SEQ ID NO: 212 |
| AAVhu.64 See US20150315612 SEQ ID NO: 196 | AAVhu.66 See US20150315612 SEQ ID NO: 197 |
| AAVhu.67 See US20150315612 SEQ ID NO: 215 | AAVhu.67 See US20150315612 SEQ ID NO: 198 |
| AAVhu.7 See US20150315612 SEQ ID NO: 226 | AAVhu.7 See US20150315612 SEQ ID NO: 150 |
| AAVhu.7 (AAV7.3) See US20150315612 SEQ ID NO: 55 | AAVhu.71 See US20150315612 SEQ ID NO: 79 |
| AAVhu.8 See US20150315612 SEQ ID NO: 53 | AAVhu.8 See US20150315612 SEQ ID NO: 12 |
| AAVhu.8 See US20150315612 SEQ ID NO: 151 | AAVhu.9 (AAV3.1) See US20150315612 SEQ ID NO: 58 |
| AAVhu.9 (AAV3.1) See US20150315612 SEQ ID NO: 155 | AAV-LK01 See US20150376607 SEQ ID NO: 2 |
| AAV-LK01 See US20150376607 SEQ ID NO: 29 | AAV-LK02 See US20150376607 SEQ ID NO: 3 |
| AAV-LK02 See US20150376607 SEQ ID NO: 30 | AAV-LK03 See US20150376607 SEQ ID NO: 4 |
| AAV-LK03 See WO2015121501 SEQ ID NO: 12, US20150376607 SEQ ID NO: 31 | |
| AAV-LK04 See US20150376607 SEQ ID NO: 5 | AAV-LK04 See US20150376607 SEQ ID NO: 32 |
| AAV-LK05 See US20150376607 SEQ ID NO: 6 | AAV-LK05 See US20150376607 SEQ ID NO: 33 |
| AAV-LK06 See US20150376607 SEQ ID NO: 7 | AAV-LK06 See US20150376607 SEQ ID NO: 34 |
| AAV-LK07 See US20150376607 SEQ ID NO: 8 | AAV-LK07 See US20150376607 SEQ ID NO: 35 |
| AAV-LK08 See US20150376607 SEQ ID NO: 9 | AAV-LK08 See US20150376607 SEQ ID NO: 36 |
| AAV-LK09 See US20150376607 SEQ ID NO: 10 | AAV-LK09 See US20150376607 SEQ ID NO: 37 |
| AAV-LK10 See US20150376607 SEQ ID NO: 11 | AAV-LK10 See US20150376607 SEQ ID NO: 38 |
| AAV-LK11 See US20150376607 SEQ ID NO: 12 | AAV-LK11 See US20150376607 SEQ ID NO: 39 |
| AAV-LK12 See US20150376607 SEQ ID NO: 13 | AAV-LK12 See US20150376607 SEQ ID NO: 40 |
| AAV-LK13 See US20150376607 SEQ ID NO: 14 | AAV-LK13 See US20150376607 SEQ ID NO: 41 |
| AAV-LK14 See US20150376607 SEQ ID NO: 15 | AAV-LK14 See US20150376607 SEQ ID NO: 42 |
| AAV-LK15 See US20150376607 SEQ ID NO: 16 | AAV-LK15 See US20150376607 SEQ ID NO: 43 |
| AAV-LK16 See US20150376607 SEQ ID NO: 17 | AAV-LK16 See US20150376607 SEQ ID NO: 44 |
| AAV-LK17 See US20150376607 SEQ ID NO: 18 | AAV-LK17 See US20150376607 SEQ ID NO: 45 |
| AAV-LK18 See US20150376607 SEQ ID NO: 19 | AAV-LK18 See US20150376607 SEQ ID NO: 46 |
| AAV-LK19 See US20150376607 SEQ ID NO: 20 | AAV-LK19 See US20150376607 SEQ ID NO: 47 |
| AAV-PAEC See US20150376607 SEQ ID NO: 1 | AAV-PAEC See US20150376607 SEQ ID NO: 48 |
| AAV-PAEC11 See US20150376607 SEQ ID NO: 26 | AAV-PAEC11 See US20150376607 SEQ ID NO: 54 |
| AAV-PAEC 12 See US20150376607 SEQ ID NO: 27 | AAV-PAEC 12 See US20150376607 SEQ ID NO: 51 |
| AAV-PAEC 13 See US20150376607 SEQ ID NO: 28 | AAV-PAEC 13 See US20150376607 SEQ ID NO: 49 |
| AAV-PAEC2 See US20150376607 SEQ ID NO: 21 | AAV-PAEC2 See US20150376607 SEQ ID NO: 56 |
| AAV-PAEC4 See US20150376607 SEQ ID NO: 22 | AAV-PAEC4 See US20150376607 SEQ ID NO: 55 |
| AAV-PAEC6 See US20150376607 SEQ ID NO: 23 | AAV-PAEC6 See US20150376607 SEQ ID NO: 52 |
| AAV-PAEC7 See US20150376607 SEQ ID NO: 24 | AAV-PAEC7 See US20150376607 SEQ ID NO: 53 |
| AAV-PAEC8 See US20150376607 SEQ ID NO: 25 | AAV-PAEC8 See US20150376607 SEQ ID NO: 50 |
| AAVpi.1 See US20150315612 SEQ ID NO: 28 | AAVpi.1 See US20150315612 SEQ ID NO: 93 AAVpi.2 408 US20150315612 SEQ ID NO: 30 |
| AAVpi.2 See US20150315612 SEQ ID NO: 95 | AAVpi.3 See US20150315612 SEQ ID NO: 29 |
| AAVpi.3 See US20150315612 SEQ ID NO: 94 | AAVrh.10 See US20150159173 SEQ ID NO: 9 |
| AAVrh.10 See US20150159173 SEQ ID NO: 25 | AAV44.2 See US20030138772 SEQ ID NO: 59 |
| AAVrh.10 (AAV44.2) See US20030138772 SEQ ID NO: 81 | AAV42.1B See US20030138772 SEQ ID NO: 90 |
| AAVrh.l2 (AAV42.1b) See US20030138772 SEQ ID NO: 30 | AAVrh.13 See US20150159173 SEQ ID NO: 10 |
| AAVrh.13 See US20150159173 SEQ ID NO: 26 | AAVrh.13 See US20150315612 SEQ ID NO: 228 |
| AAVrh.l3R See US20150159173 | AAV42.3A See US20030138772 SEQ ID NO: 87 |
| AAVrh.l4 (AAV42.3a) See US20030138772 SEQ ID NO: 32 | AAV42.5A See US20030138772 SEQ ID NO: 89 |
| AAVrh.l7 (AAV42.5a) See US20030138772 SEQ ID NO: 34 | AAV42.5B See US20030138772 SEQ ID NO: 91 |
| AAVrh.l8 (AAV42.5b) See US20030138772 SEQ ID NO: 29 | AAV42.6B See US20030138772 SEQ ID NO: 112 |
| AAVrh.l9 (AAV42.6b) See US20030138772 SEQ ID NO: 38 | AAVrh.2 See US20150159173 SEQ ID NO: 39 |
| AAVrh.2 See US20150315612 SEQ ID NO: 231 | AAVrh.20 See US20150159173 SEQ ID NO: 1 |
| AAV42.10 See US20030138772 SEQ ID NO: 106 | AAVrh.21 (AAV42.10) See US20030138772 SEQ ID NO: 35 |
| AAV42.11 See US20030138772 SEQ ID NO: 108 | AAVrh.22 (AAV42.11) See US20030138772 SEQ ID NO: 37 |
| AAV42.12 See US20030138772 SEQ ID NO: 113 | AAVrh.23 (AAV42.12) See US20030138772 SEQ ID NO: 58 |
| AAV42.13 See US20030138772 SEQ ID NO: 86 | AAVrh.24 (AAV42.13) See US20030138772 SEQ ID NO: 31 |
| AAV42.15 See US20030138772 SEQ ID NO: 84 | AAVrh.25 (AAV42.15) See US20030138772 SEQ ID NO: 28 |
| AAVrh.2R See US20150159173 | AAVrh.31 (AAV223.1) See US20030138772 SEQ ID NO: 48 |
| AAVC1 See US20030138772 SEQ ID NO: 60 | AAVrh.32 (AAVC1) See 446 US20030138772 SEQ ID NO: 19 |
| AAVrh.32/33 See US20150159173 SEQ ID NO: 2 | AAVrh.51 (AAV2-5) See US20150315612 SEQ ID NO: 104 |
| AAVrh.52 (AAV3-9) See US20150315612 SEQ ID NO: 18 | AAVrh.52 (AAV3-9) See US20150315612 SEQ ID NO: 96 |
| AAVrh.53 See US20150315612 SEQ ID NO: 97 | AAVrh.53 (AAV3-11) See US20150315612 SEQ ID NO: 17 |
| AAVrh.53 (AAV3-11) See US20150315612 SEQ ID NO: 186 | AAVrh.54 See US20150315612 SEQ ID NO: 40 |
| AAVrh.54 See US20150159173 SEQ ID NO: 49, US20150315612 SEQ ID NO: 116 | |
| AAVrh.55 See US20150315612 SEQ ID NO: 37 | AAVrh.55 (AAV4-19) See US20150315612 SEQ ID NO: 117 |
| AAVrh.56 v US20150315612 SEQ ID NO: 54 | AAVrh.56 See US20150315612 SEQ ID NO: 152 |
| AAVrh.57 See 497 US20150315612 SEQ ID NO: 26 | AAVrh.57 See US20150315612 SEQ ID NO: 105 |
| AAVrh.58 See US20150315612 SEQ ID NO: 27 | AAVrh.58 See US20150159173 SEQ ID NO: 48, US20150315612 SEQ ID NO: 106 |
| | AAVrh.58 See US20150315612 SEQ ID NO: 232 |
| AAVrh.59 See US20150315612 SEQ ID NO: 42 | AAVrh.59 See US20150315612 SEQ ID NO: 110 |
| AAVrh.60 See US20150315612 SEQ ID NO: 31 | AAVrh.60 See US20150315612 SEQ ID NO: 120 |
| AAVrh.61 See US20150315612 SEQ ID NO: 107 | AAVrh.61 (AAV2-3) See US20150315612 SEQ ID NO: 21 |
| AAVrh.62 (AAV2-15) See US20150315612 SEQ ID No: 33 | AAVrh.62 (AAV2-15) See US20150315612 SEQ ID NO: 114 |
| AAVrh.64 See US20150315612 SEQ ID No: 15 | AAVrh.64 See US20150159173 SEQ ID NO: 43, US20150315612 SEQ ID NO: 99 |
| | AAVrh.64 See US20150315612 SEQ ID NO: 233 |
| AAVRh.64Rl See US20150159173 | AAVRh.64R2 See US20150159173 |
| AAVrh.65 See US20150315612 SEQ ID NO: 35 | AAVrh.65 See US20150315612 SEQ ID NO: 112 |
| AAVrh.67 See US20150315612 SEQ ID NO: 36 | AAVrh.67 See US20150315612 SEQ ID NO: 230 |
| AAVrh.67 See US20150159173 SEQ ID NO: 47, US20150315612 SEQ ID NO: 113 | |
| AAVrh.68 See US20150315612 SEQ ID NO: 16 | AAVrh.68 See US20150315612 SEQ ID NO: 100 |
| AAVrh.69 See US20150315612 SEQ ID NO: 39 | AAVrh.69 See US20150315612 SEQ ID NO: 119 |
| AAVrh.70 See US20150315612 SEQ ID NO: 20 | AAVrh.70 See US20150315612 SEQ ID NO: 98 |
| AAVrh.71 See US20150315612 SEQ ID NO: 162 | AAVrh.72 See US20150315612 SEQ ID NO: 9 |
| AAVrh.73 See US20150159173 SEQ ID NO: 5 | AAVrh.74 See US20150159173 SEQ ID NO: 6 |
| AAVrh.8 See US20150159173 SEQ ID NO: 41 | AAVrh.8 See US20150315612 SEQ ID NO: 235 |
| AAVrh.8R See US20150159173, WO2015168666 SEQ ID NO: 9 | AAVrh.8R A586R mutant See WO2015168666 SEQ ID NO: 10 |
| AAVrh.8R R533A mutant See WO2015168666 SEQ ID NO: 11 | BAAV (bovine AAV) See US9193769 SEQ ID NO: 8 |
| BAAV (bovine AAV) See US9193769 SEQ ID NO: 10 | BAAV (bovine AAV) See US9193769 SEQ ID NO: 4 |
| BAAV (bovine AAV) See US9193769 SEQ ID NO: 2 | BAAV (bovine AAV) See US9193769 SEQ ID NO: 6 |
| BAAV (bovine AAV) See US9193769 SEQ ID NO: 1 | BAAV (bovine AAV) See US9193769 SEQ ID NO: 5 |
| BAAV (bovine AAV) See US9193769 SEQ ID NO: 3 | BAAV (bovine AAV) See US9193769 SEQ ID NO: 11 |
| BAAV (bovine AAV) See US7427396 SEQ ID NO: 5 | BAAV (bovine AAV) See US7427396 SEQ ID NO: 6 |
| BAAV (bovine AAV) See US9193769 SEQ ID NO: 7 | BAAV (bovine AAV) See US9193769 SEQ ID NO: 9 |
| BNP61 AAV See US20150238550 SEQ ID NO: 1 | BNP61 AAV See US20150238550 SEQ ID NO: 2 |
| BNP62 AAV See US20150238550 SEQ ID NO: 3 | BNP63 AAV See US20150238550 SEQ ID NO: 4 |
| caprine AAV See US7427396 SEQ ID NO: 3 | caprine AAV See US7427396 SEQ ID NO: 4 |
| true type AAV (ttAAV) See WO2015121501 SEQ ID NO: 2 | AAAV (Avian AAV) See US9238800 SEQ ID NO: 12 |
| AAAV (Avian AAV) See US9238800 SEQ ID NO: 2 | AAAV (Avian AAV) See US9238800 SEQ ID NO: 6 |
| AAAV (Avian AAV) See US9238800 SEQ ID NO: 4 | AAAV (Avian AAV) See US9238800 SEQ ID NO: 8 |
| AAAV (Avian AAV) See US9238800 SEQ ID NO: 14 | AAAV (Avian AAV) See US9238800 SEQ ID NO: 10 |
| AAAV (Avian AAV) See US9238800 SEQ ID NO: 15 | AAAV (Avian AAV) See US9238800 SEQ ID NO: 5 |
| AAAV (Avian AAV) See US9238800 SEQ ID NO: 9 | AAAV (Avian AAV) See US9238800 SEQ ID NO: 3 |
| AAAV (Avian AAV) See US9238800 SEQ ID NO: 7 | AAAV (Avian AAV) See US9238800 SEQ ID NO: 11 |
| AAAV (Avian AAV) See US9238800 SEQ ID NO: 13 | AAAV (Avian AAV) See US9238800 SEQ ID NO: 1 |
| AAV Shuffle 100-1 See US20160017295 SEQ ID NO: 23 | AAV Shuffle 100-1 See US20160017295 SEQ ID NO: 11 |
| AAV Shuffle 100-2 See US20160017295 SEQ ID NO: 37 | AAV Shuffle 100-2 See US20160017295 SEQ ID NO: 29 |
| AAV Shuffle 100-3 See US20160017295 SEQ ID NO: 24 | AAV Shuffle 100-3 See US20160017295 SEQ ID NO: 12 |
| AAV Shuffle 100-7 See US20160017295 SEQ ID NO: 25 | AAV Shuffle 100-7 See US20160017295 SEQ ID NO: 13 |
| AAV Shuffle 10-2 See US20160017295 SEQ ID NO: 34 | AAV Shuffle 10-2 See US20160017295 SEQ ID NO: 26 |
| AAV Shuffle 10-6 See US20160017295 SEQ ID NO: 35 | AAV Shuffle 10-6 See US20160017295 SEQ ID NO: 27 |
| AAV Shuffle 10-8 See US20160017295 SEQ ID NO: 36 | AAV Shuffle 10-8 See US20160017295 SEQ ID NO: 28 |
| AAV SM 100-10 See US20160017295 SEQ ID NO: 41 | AAV SM 100-10 See US20160017295 SEQ ID NO: 33 |
| AAV SM 100-3 See US20160017295 SEQ ID NO: 40 | AAV SM 100-3 See US20160017295 SEQ ID NO: 32 |
| AAV SM 10-1 See US20160017295 SEQ ID NO: 38 | AAV SM 10-1 See US20160017295 SEQ ID NO: 30 |
| AAV SM 10-2 See US20160017295 SEQ ID NO: 10 | AAV SM 10-2 See US20160017295 SEQ ID NO: 22 |
| AAV SM 10-8 See US20160017295 SEQ ID NO: 39 | AAV SM 10-8 See US20160017295 SEQ ID NO: 31 |
| AAV CBr-7.1 See WO2016065001 SEQ ID NO: 4 | AAV CBr-7.1 See WO2016065001 SEQ ID NO: 54 |
| AAV CBr-7.10 See WO2016065001 SEQ ID NO: 11 | AAV CBr-7.10 See WO2016065001 SEQ ID NO: 61 |
| AAV CBr-7.2 See WO2016065001 SEQ ID NO: 5 | AAV CBr-7.2 See WO2016065001 SEQ ID NO: 55 |
| AAV CBr-7.3 See WO2016065001 SEQ ID NO: 6 | AAV CBr-7.3 See WO2016065001 SEQ ID NO: 56 |
| AAV CBr-7.4 See WO2016065001 SEQ ID NO: 7 | AAV CBr-7.4 See WO2016065001 SEQ ID NO: 57 |
| AAV CBr-7.5 See WO2016065001 SEQ ID NO: 8 | AAV CHt-6.6 See WO2016065001 SEQ ID NO: 35 |
| AAV CHt-6.6 See WO2016065001 SEQ ID NO: 85 | AAV CHt-6.7 See WO2016065001 SEQ ID NO: 36 |
| AAV CHt-6.7 See WO2016065001 SEQ ID NO: 86 | AAV CHt-6.8 See WO2016065001 SEQ ID NO: 37 |
| AAV CHt-6.8 See WO2016065001 SEQ ID NO: 87 | AAV CHt-P1 See WO2016065001 SEQ ID NO: 29 |
| AAV CHt-P1 See WO2016065001 SEQ ID NO: 79 | AAV CHt-P2 See WO2016065001 SEQ ID NO: 1 |
| AAV CHt-P2 See WO2016065001 SEQ ID NO: 51 | AAV CHt-P5 See WO2016065001 SEQ ID NO: 2 |
| AAV CHt-P5 See WO2016065001 SEQ ID NO: 52 | AAV CHt-P6 See WO2016065001 SEQ ID NO: 30 |
| AAV CHt-P6 See WO2016065001 SEQ ID NO: 80 | AAV CHt-P8 See WO2016065001 SEQ ID NO: 31 |
| AAV CHt-P8 See WO2016065001 SEQ ID NO: 81 | AAV CHt-P9 See WO2016065001 SEQ ID NO: 3 |
| AAV CHt-P9 See WO2016065001 SEQ ID NO: 53 | AAV CKd-1 See US8734809 SEQ ID NO 57 |
| AAV CKd-1 See US8734809 SEQ ID NO 131 | AAV CKd-10 See US8734809 SEQ ID NO 58 |
| AAV CKd-10 See US8734809 SEQ ID NO 132 | AAV CKd-2 See US8734809 SEQ ID NO 59 |
| AAV CKd-2 See US8734809 SEQ ID NO 133 | AAV CKd-3 See US8734809 SEQ ID NO 60 |
| AAV CKd-3 See US8734809 SEQ ID NO 134 | AAV CKd-4 See US8734809 SEQ ID NO 61 |
| AAVCKd-4 See US8734809 SEQ ID NO 135 | AAV CKd-6 See US8734809 SEQ ID NO 62 |
| AAV CKd-6 See US8734809 SEQ ID NO 136 | AAV CKd-7 See US8734809 SEQ ID NO 63 |
| AAV CKd-7 See US8734809 SEQ ID NO 137 | AAV CKd-8 See US8734809 SEQ ID NO 64 |
| AAVCKd-8 See US8734809 SEQ ID NO 138 | AAV CKd-B 1 See US8734809 SEQ ID NO 73 |
| AAV CKd-B 1 See US8734809 SEQ ID NO 147 | AAV CKd-B2 See US8734809 SEQ ID NO 74 |
| AAV CKd-B2 See US8734809 SEQ ID NO 148 | AAV CKd-B3 See US8734809 SEQ ID NO 75 |
| AAV CKd-B3 See US8734809 | AAV CKd-B3 See US8734809 SEQ ID NO 149 |
| AAV CLv-1 See US8734809 SEQ ID NO: 65 | AAV CLv-1 See US8734809 SEQ ID NO: 139 |
| AAV CLvl-1 See US8734809 SEQ ID NO: 171 | AAV Civ 1-10 See US8734809 SEQ ID NO: 178 |
| AAV CLvl-2 See US8734809 SEQ ID NO: 172 | AAV CLv-12 See US8734809 SEQ ID NO: 66 |
| AAV CLv-12 See US8734809 SEQ ID NO: 140 | AAV CLvl-3 See US8734809 SEQ ID NO: 173 |
| AAV CLv-13 See US8734809 SEQ ID NO: 67 | AAV CLv-13 See US8734809 SEQ ID NO: 141 |
| AAV CLvl-4 See US8734809 SEQ ID NO: 174 | AAV Civ 1-7 See US8734809 SEQ ID NO: 175 |
| AAV Civ 1-8 See US8734809 SEQ ID NO: 176 | AAV Civ 1-9 See US8734809 SEQ ID NO: 177 |
| AAV CLv-2 See US8734809 SEQ ID NO: 68 | AAV CLv-2 See US8734809 SEQ ID NO: 142 |
| AAV CLv-3 See US8734809 SEQ ID NO: 69 | AAV CLv-3 See US8734809 SEQ ID NO: 143 |
| AAV CLv-4 See US8734809 SEQ ID NO: 70 | AAV CLv-4 See US8734809 SEQ ID NO: 144 |
| AAV CLv-6 See US8734809 SEQ ID NO: 71 | AAV CLv-6 See US8734809 SEQ ID NO: 145 |
| AAV CLv-8 See US8734809 SEQ ID NO: 72 | AAV CLv-8 See US8734809 SEQ ID NO: 146 |
| AAV CLv-D1 See US8734809 SEQ ID NO: 22 | AAV CLv-D1 See US8734809 SEQ ID NO: 96 |
| AAV CLv-D2 See US8734809 SEQ ID NO: 23 | AAV CLv-D2 See US8734809 SEQ ID NO: 97 |
| AAV CLv-D3 See US8734809 SEQ ID NO: 24 | AAV CLv-D3 See US8734809 SEQ ID NO: 98 |
| AAV CLv-D4 See US8734809 SEQ ID NO: 25 | AAV CLv-D4 See US8734809 SEQ ID NO: 99 |
| AAV CLv-D5 See US8734809 SEQ ID NO: 26 | AAV CLv-D5 See US8734809 SEQ ID NO: 100 |
| AAV CLv-D6 See US8734809 SEQ ID NO: 27 | AAV CLv-D6 See US8734809 SEQ ID NO: 101 |
| AAV CLv-D7 See US8734809 SEQ ID NO: 28 | AAV CLv-D7 See US8734809 SEQ ID NO: 102 |
| AAV CLv-D8 See US8734809 SEQ ID NO: 29 | AAV CLv-D8 See US8734809 SEQ ID NO: 103 AAV CLv-K1 762 WO2016065001 SEQ ID NO: 18 |
| AAV CLv-K1 See WO2016065001 SEQ ID NO: 68 | AAV CLv-K3 See WO2016065001 SEQ ID NO: 19 |
| AAV CLv-K3 See WO2016065001 SEQ ID NO: 69 | AAV CLv-K6 See WO2016065001 SEQ ID NO: 20 |
| AAV CLv-K6 See WO2016065001 SEQ ID NO: 70 | AAV CLv-L4 See WO2016065001 SEQ ID NO: 15 |
| AAV CLv-L4 See WO2016065001 SEQ ID NO: 65 | AAV CLv-L5 See WO2016065001 SEQ ID NO: 16 |
| AAV CLv-L5 See WO2016065001 SEQ ID NO: 66 | AAV CLv-L6 See WO2016065001 SEQ ID NO: 17 |
| AAV CLv-L6 See WO2016065001 SEQ ID NO: 67 | AAV CLv-M1 See WO2016065001 SEQ ID NO: 21 |
| AAV CLv-Ml See WO2016065001 SEQ ID NO: 71 | AAV CLv-Mll See WO2016065001 SEQ ID NO: 22 |
| AAV CLv-Ml 1 See WO2016065001 SEQ ID NO: 72 | AAV CLv-M2 See WO2016065001 SEQ ID NO: 23 |
| AAV CLv-M2 See WO2016065001 SEQ ID NO: 73 | AAV CLv-M5 See WO2016065001 SEQ ID NO: 24 |
| AAV CLv-M5 See WO2016065001 SEQ ID NO: 74 | AAV CLv-M6 See WO2016065001 SEQ ID NO: 25 |
| AAV CLv-M6 See WO2016065001 SEQ ID NO: 75 | AAV CLv-M7 See WO2016065001 SEQ ID NO: 26 |
| AAV CLv-M7 See WO2016065001 SEQ ID NO: 76 | AAV CLv-M8 See WO2016065001 SEQ ID NO: 27 |
| AAV CLv-M8 See WO2016065001 SEQ ID NO: 77 | AAV CLv-M9 See WO2016065001 SEQ ID NO: 28 |
| AAV CLv-M9 See WO2016065001 SEQ ID NO: 78 | AAV CLv-R1 See US8734809 SEQ ID NO 30 |
| AAV CLv-Rl See US8734809 SEQ ID NO 104 | AAV CLv-R2 See US8734809 SEQ ID NO 31 |
| AAV CLv-R2 See US8734809 SEQ ID NO 105 | AAV CLv-R3 See US8734809 SEQ ID NO 32 |
| AAV CLv-R3 See US8734809 SEQ ID NO 106 | AAV CLv-R4 See US8734809 SEQ ID NO 33 |
| AAV CLv-R4 See US8734809 SEQ ID NO 107 | AAV CLv-R5 See US8734809 SEQ ID NO 34 |
| AAV CLv-R5 See US8734809 SEQ ID NO 108 | AAV CLv-R6 See US8734809 SEQ ID NO 35 |
| AAV CLv-R6 See US8734809 SEQ ID NO 109 AAV CLv-R7 802 US8734809 SEQ ID NO 36 | AAV CLv-R7 See US8734809 SEQ ID NO 110 |
| AAV CLv-R8 See US8734809 SEQ ID NO 37 | AAV CLv-R8 See US8734809 SEQ ID NO 111 |
| AAV CLv-R9 See US8734809 SEQ ID NO 38 | AAV CLv-R9 See US8734809 SEQ ID NO 112 |
| AAV CSp-1 See US8734809 SEQ ID NO 45 | AAV CSp-1 See US8734809 SEQ ID NO 119 |
| AAV CSp-10 See US8734809 SEQ ID NO 46 | AAV CSp-10 See US8734809 SEQ ID NO 120 |
| AAV CSp-11 See US8734809 SEQ ID NO 47 | AAV CSp-11 See US8734809 SEQ ID NO 121 |
| AAV CSp-2 See US8734809 SEQ ID NO 48 | AAV CSp-2 See US8734809 SEQ ID NO 122 |
| AAV CSp-3 See US8734809 SEQ ID NO 49 | AAV CSp-3 See US8734809 SEQ ID NO 123 |
| AAV CSp-4 See US8734809 SEQ ID NO 50 | AAV CSp-4 See US8734809 SEQ ID NO 124 |
| AAV CSp-6 See US8734809 SEQ ID NO 51 | AAV CSp-6 See US8734809 SEQ ID NO 125 |
| AAV CSp-7 See US8734809 SEQ ID NO 52 | AAV CSp-7 See US8734809 SEQ ID NO 126 |
| AAV CSp-8 See US8734809 SEQ ID NO 53 | AAV CSp-8 See US8734809 SEQ ID NO 127 |
| AAV CSp-8.10 See WO2016065001 SEQ ID NO: 38 | AAV CSp-8.10 See WO2016065001 SEQ ID NO: 88 |
| AAV CSp-8.2 See WO2016065001 SEQ ID NO: 39 | AAV CSp-8.2 See WO2016065001 SEQ ID NO: 89 |
| AAV CSp-8.4 See WO2016065001 SEQ ID NO: 40 | AAV CSp-8.4 See WO2016065001 SEQ ID NO: 90 |
| AAV CSp-8.5 See WO2016065001 SEQ ID NO: 41 | AAV CSp-8.5 See WO2016065001 SEQ ID NO: 91 |
| AAV CSp-8.6 See WO2016065001 SEQ ID NO: 42 | AAV CSp-8.6 See WO2016065001 SEQ ID NO: 92 |
| AAV CSp-8.7 See WO2016065001 SEQ ID NO: 43 | AAV CSp-8.7 See WO2016065001 SEQ ID NO: 93 |
| AAV CSp-8.8 See WO2016065001 SEQ ID NO: 44 | AAV CSp-8.8 See WO2016065001 SEQ ID NO: 94 |
| AAV CSp-8.9 See WO2016065001 SEQ ID NO: 45 | AAV CSp-8.9 See WO2016065001 SEQ ID NO: 95 |
| AAV CSp-9 842 US8734809 SEQ ID NO: 54 | AAV CSp-9 See US8734809 SEQ ID NO: 128 |
| AAV.hu.48R3 See US8734809 SEQ ID NO: 183 | AAV.VR-355 See US8734809 SEQ ID NO: 181 |
| AAV3B See WO2016065001 SEQ ID NO: 48 | AAV3B See WO2016065001 SEQ ID NO: 98 |
| AAV4 See WO2016065001 SEQ ID NO: 49 | AAV4 See WO2016065001 SEQ ID NO: 99 |
| AAV5 See WO2016065001 SEQ ID NO: 50 | AAV5 See WO2016065001 SEQ ID NO: 100 |
| AAVF1/HSC1 See WO2016049230 SEQ ID NO: 20 | AAVF1/HSC1 See WO2016049230 SEQ ID NO: 2 |
| AAVF11/HSC11 See WO2016049230 SEQ ID NO: 26 | AAVF11/HSC11 See WO2016049230 SEQ ID NO: 4 |
| AAVF12/HSC12 See WO2016049230 SEQ ID NO: 30 | AAVF12/HSC12 See WO2016049230 SEQ ID NO: 12 |
| AAVF13/HSC13 See WO2016049230 SEQ ID NO: 31 | AAVF13/HSC13 See WO2016049230 SEQ ID NO: 14 |
| AAVF14/HSC14 See WO2016049230 SEQ ID NO: 32 | AAVF14/HSC14 See WO2016049230 SEQ ID NO: 15 |
| AAVF15/HSC15 See WO2016049230 SEQ ID NO: 33 | AAVF15/HSC15 See WO2016049230 SEQ ID NO: 16 |
| AAVF16/HSC16 See WO2016049230 SEQ ID NO: 34 | AAVF16/HSC16 See WO2016049230 SEQ ID NO: 17 |
| AAVF17/HSC17 See WO2016049230 SEQ ID NO: 35 | AAVF17/HSC17 See WO2016049230 SEQ ID NO: 13 |
| AAVF2/HSC2 See WO2016049230 SEQ ID NO: 21 | AAVF2/HSC2 See WO2016049230 SEQ ID NO: 3 |
| AAVF3/HSC3 See WO2016049230 SEQ ID NO: 22 | AAVF3/HSC3 See WO2016049230 SEQ ID NO: 5 |
| AAVF4/HSC4 See WO2016049230 SEQ ID NO: 23 | AAVF4/HSC4 See WO2016049230 SEQ ID NO: 6 |
| AAVF5/HSC5 See WO2016049230 SEQ ID NO: 25 | AAVF5/HSC5 See WO2016049230 SEQ ID NO: 11 |
| AAVF6/HSC6 See WO2016049230 SEQ ID NO: 24 | AAVF6/HSC6 See WO2016049230 SEQ ID NO: 7 |
| AAVF7/HSC7 See WO2016049230 SEQ ID NO: 27 | AAVF7/HSC7 See WO2016049230 SEQ ID NO: 8 |
| AAVF8/HSC8 See WO2016049230 SEQ ID NO: 28 | AAVF8/HSC8 See WO2016049230 SEQ ID NO: 9 |
| | AAVF9/HSC9 882 WO2016049230 SEQ ID NO: 29 |
| AAVF9/HSC9 See WO2016049230 SEQ ID NO: 10 | |

There are various ways to achieve selective expression of a viral vector-delivered transgene in a target cell. These include, for example, selecting a virus with a tropism that favors infection of a given cell type, placing the transgene under control of a tissue-specific or cell-type specific promoter or regulatory element(s), locally delivering the viral vector, or a combination of any of these.

Tropism for various viral vectors is known in the art, such that one of ordinary skill in the art can choose a viral vector, e.g., an AAV vector, known to display a preference for infection of the given target tissue or cell type. For a detailed review, see, e.g., Castle et al., Methods Mo. Biol. 1382: 133-149 (2016), which is incorporated herein by reference in its entirety and discusses the properties of a range of AAV variants and engineered capsids, and provides a guide for selecting the appropriate vector for specific applications in the CNS and retina; see also, e.g., Ogden et al., Science 366: 1139-1143 (2019).

In addition to selecting a naturally occurring or mutated AAV variant that tends to infect a desired cell type or tissue, tropism can also be modified, e.g., by chemically modifying the capsid to include one or more moieties that influence the tropism of the virus. See, e.g., PCT/EP2017/064089 and US 20190153474, each of which is incorporated herein by reference in its entirety, and which describe chemical modifications to the AAV capsid that influence CNS tropism of the viral particles.

In some embodiments of any of the aspects described herein, the sequence encoding the TGF-β polypeptide is operably linked to a neuronal tissue-specific promoter, including but not limited to a retina-specific promoter as described herein. In some embodiments of any of the aspects described herein, the sequence encoding the TGF-β polypeptide is operably linked to a retinal-specific promoter.

Promoters or regulatory elements that direct expression in a given tissue or cell-type are known in the art. Non-limiting examples of interest, e.g., for driving retinal-specific expression of a transgene include the rhodopsin kinase promoter, which is active in both rods and cones (see, e.g., Sun et al., Gene Ther. 17: 117-131 (2010)), and the opsin promoters (driving expression of S(blue), M (green) and L (red) opsin photopigment genes (see, e.g., Li et al., Vision Res. 48: 332-338 (2008)), which are active in cones. Where one desires to express a transgene, such as a transgene encoding a TGF-β polypeptide, in a microglial cell, a microglial cell specific promoter such as a P2Y purinoceptor 12 promoter or a Transmembrane protein 119 (TMEM119) promoter can be used. The full length and promoter sequences for P2Y and TMEM119 are known in the art.

In some embodiments, a promoter active in microglial cells can be used, for example, to direct expression of a transgene in a population of retinal microglial cells. Promoters active in microglial cells include, but are not limited to the P2Y purinoceptor 12 (P2Y12; also known as P2YR12) promoter, and the Transmembrane protein 119 (TMEM119) promoter. Additional examples of microglial-specific promoters are described, e.g., in Cserép C, Pósfai B, Lénárt N, Fekete R, László ZI, Lele Z, et al. (January 2020). “Microglia monitor and protect neuronal function through specialized somatic purinergic junctions”. Science. 367 (6477): 528-537; and Cucchiarini et al., Gene Therapy (2003) 10, 657-667, the content of each of which is incorporated herein by reference in their entireties. Sequences for the P2Y12 gene and transcripts are known in the art for human and non-human species, e.g., purinergic receptor P2Y12 [Homo sapiens (human)]: NCBI reference Gene ID: 64805; Promoters for orthologous P2Y12 receptor genes known in the art can also be used to the extent that they drive microglial expression of, e.g., a TGF-β-encoding transgene. Sequences for the TMEM119 gene and transcripts are known in the art for human and non-human species, e.g., transmembrane protein 119 [Homo sapiens (human)]: NCBI reference Gene ID: 338773; Promoters for orthologous TMEM119 receptor genes known in the art can also be used to the extent that they drive microglial expression of, e.g., a TGF-β-encoding transgene.

The genomic sequence for Homo sapiens P2Y12 (P2RY12), at position c151384753-151336843 on chromosome 3, includes the promoter that provides microglia-specific expression and is provided in NCBI Reference No. NC_000003.12. The genomic sequence for Homo sapiens TMEM119, located at position c108598099-108589846 on chromosome 12, includes the promoterthat provides microglia-specific expression and is provided in NCBI Reference Sequence: NC_0.0012.12.

In some embodiments of any of the aspects, the retinal-specific promoter described herein is the red opsin promoter. The sequence for the human red opsin promoter sequence is known in the art, e.g., NCBI Reference ID KT886395.1.

### Homo Sapiens Clone PR1.7 Red Cone Opsin Gene, Promoter Region and Partial Cds Sequence ID: KT886395.1

cctacagcagccagggtgagattatgaggctgagctgagaatatcaagac
tgtaccgagtagggggccttggcaagtgtggagagcccggcagctggggc
agagggcggagtacggtgtgcgtttacggacctcttcaaacgaggtagga
aggtcagaagtcaaaaagggaacaaatgatgtttaaccacacaaaaatga
aaatccaatggttggatatccattccaaatacacaaaggcaacggataag
tgatccgggccaggcacagaaggccatgcacccgtaggattgcactcaga
gctcccaaatgcataggaatagaagggtgggtgcaggaggctgaggggtg
gggaaagggcatgggtgtttcatgaggacagagcttccgtttcatgcaat
gaaaagagtttggagacggatggtggtgactggactatacacttacacac
ggtagcgatggtacactttgtattatgtatattttaccacgatcttttta
aagtgtcaaaggcaaatggccaaatggttccttgtcctatagctgtagca
gccatcggctgttagtgacaaagcccctgagtcaagatgacagcagcccc
cataactcctaatcggctctcccgcgtggagtcatttaggagtagtcgca
ttagagacaagtccaacatctaatcttccaccctggccagggccccagct
ggcagcgagggtgggagactccgggcagagcagagggcgctgacattggg
gcccggcctggcttgggtccctctggcctttccccaggggccctctttcc
ttggggctttcttgggccgccactgctcccgctcctctccccccatccca
ccccctcaccccctcgttcttcatatccttctctagtgctccctccactt
tcatccacccttctgcaagagtgtgggaccacaaatgagttttcacctgg
cctggggacacacgtgcccccacaggtgctgagtgactttctaggacagt
aatctgctttaggctaaaatgggacttgatcttctgttagccctaatcat
caattagcagagccggtgaaggtgcagaacctaccgcctttccaggcctc
ctcccacctctgccacctccactctccttcctgggatgtgggggctggca
cacgtgtggcccagggcattggtgggattgcactgagctgggtcattagc
gtaatcctggacaagggcagacagggcgagcggagggccagctccggggc
tcaggcaaggctgggggcttcccccagacaccccactcctcctctgctgg
acccccacttcatagggcacttcgtgttctcaaagggcttccaaatagca
tggtggccttggatgcccagggaagcctcagagttgcttatctccctcta
gacagaaggggaatctcggtcaagagggagaggtcgccctgttcaaggcc
acccagccagctcatggcggtaatgggacaaggctggccagccatcccac
cctcagaagggacccggtggggcaggtgatctcagaggaggctcacttct
gggtctcacattcttggatccggttccaggcctcggccctaaatagtctc
cctgggctttcaagagaaccacatgagaaaggaggattcgggctctgagc
agtttcaccacccaccccccagtctgcaaatcctgacccgtgggtccacc
tgccccaaaggcggacgcaggacagtagaagggaacagagaacacataaa
cacagagagggccacagcggctcccacagtcaccgccaccttcctggcgg
ggatgggtggggcgtctgagtttggttcccagcaaatccctctgagccgc
ccttgcgggctcgcctcaggagcaggggagcaagaggtgggaggaggagg
tctaagtcccaggcccaattaagagatcaggtagtgtagggtttgggagc
ttttaaggtgaagaggcccgggctgatcccacaggccagtataaagcgcc
gtgaccctcaggtgatgcgccagggccggctgccgtcggggacagggctt
tccata (SEQ ID NO: 16).

In some embodiments of any of the aspects, the retina-specific promoter is the interphotoreceptor retinoid-binding protein promoter, the human transducin alpha-subunit (IRBPe/GNAT2) promoter, or human rhodopsin kinase (RK) promoter.

In some embodiments of any of the aspects, the neuronal-specific promoter is a tubulin alpha1, synapsin I, neuron-specific enolase, calcium/calmodulin-dependent protein kinase II, or platelet-derived growth factor beta chain promoter.

Additional promoters are discussed, e.g., in Dyka FM et al., “Cone specific promoter for use in gene therapy of retinal degenerative diseases.” Adv Exp Med Biol. 2014;801:695-701. doi: 10.1007/978-1-4614-3209-8_87. PMID: 24664760; PMCID: PMC4450355; Sun, X., Pawlyk, B., Xu, X. et al. “Gene therapy with a promoter targeting both rods and cones rescues retinal degeneration caused by AIPL1 mutations.” Gene Ther 17, 117-131 (2010); Elizabeth M. Simpson, et al., Human Gene Therapy. Mar 2019.257-272; Kügler S, Meyn L, et al., “Neuron-specific expression of therapeutic proteins: evaluation of different cellular promoters in recombinant adenoviral vectors.” Mol Cell Neurosci. 2001 Jan;17(1):78-96. doi: 10.1006/mcne.2000.0929. PMID: 11161471; and Hioki, H., Kameda, H., Nakamura, H. et al. Efficient gene transduction of neurons by lentivirus with enhanced neuron-specific promoters. Gene Ther 14, 872-882 (2007), the contents of each of which is incorporated herein by reference in their entireties.

Expression of AAV-encoded transgenes can optionally be improved by incorporating other transcriptional or post-transcriptional regulatory elements as known to those of skill in the art. A non-limiting example includes a woodchuck hepatitis virus post-transcriptional regulatory element (WPRE; see, e.g., Higashimoto et al., Gene Ther. 14: 1298-1304 (2007).

In some embodiments of any of the aspects, the vector comprises a WPRE sequence comprising

aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaagctga
cgtcctttccatggctgctcgcctgtgttgccacctggattctgcgcggg
acgtccttctgctacgtcccttcggccctcaatccagcggaccttccttc
ccgcggcctgctgccggctctgcggcctcttccgcgtcttcg (SEQ ID NO: 17).

In some embodiments of any of the aspects, the vector comprises a poly-A addition sequence. In some embodiments of any of the aspects, the Poly-A addition polynucleotide sequence comprises

gcctcgactgtgccttctagttgccagccatctgttgtttgcccctcccc
cgtgccttccttgaccctggaaggtgccactcccactgtcctttcctaat
aaaatgaggaaattgcatcgcattgtctgagtaggtgtcattctattctg
gggggtggggtggggcaggacagcaagggggaggattgggaagacaatag
caggcatgctgggga (SEQ ID NO: 18).

In some embodiments, the vector described herein can be a lentiviral vector. A lentiviral expression system for use in the methods described herein can further comprise long terminal repeats (LTRs) flanking the nucleic acid cassette encoding the transgene. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase into the host chromosome. LTRs and methods for manufacturing lentiviral vectors are further described, e.g., in U.S. Pat. Numbers US7083981B2; US6207455B1; US6555107B2; US8349606B2; US7262049B2; and U.S. Pat. Application Numbers US20070025970A1; US20170067079A1; US20110028694A1; the contents of each are incorporated herein by reference in their entireties.

In some embodiments, a vector for the expression of TGF-β can be an adenoviral vector. Adenoviral vectors and methods of preparing them are known in the art and described, for example, in U.S. Pats. numbered US7510875B2, US7820440B2, US7749493B2; US7820440B2, US10041049B2, International Pat. Application numbered WO2000070071A1 and WO2000070071A1, and U.S. Pat. Applications numbered US20030022356A1 and US20080050770A1, the contents of each of which are incorporated herein by reference in their entireties.

### Pharmaceutical Compositions

The methods and compositions described herein rely, in part, upon the administration of a formulation comprising a vector encoding a TGF-β polypeptide to neuronal tissue or cells affected by a neurodegenerative disease or disorder. Specific formulations and route(s) of administration will vary with the neurodegenerative disease or pathology being treated. For example, administration can comprise local administration, e.g., via injection, when the pathology is localized, e.g., as in the situation for an ocular disease, which presents opportunities for direct administration to the eye. Treatment via delivery of a TGF-β polypeptide for other CNS diseases or disorders can take other routes, such as intrathecal delivery to the spinal cord and CNS, or direct injection to the brain, e.g., to target specific locations determined to be undergoing active neurodegeneration, e.g., to target the site of active lesions in multiple sclerosis or other neurodegenerative disease.

Engineered vectors as described herein can be formulated as a pharmaceutical composition for use in the treatment of a neurodegenerative disease or disorder or an ocular disease or disorder as described herein. As used herein, the term “pharmaceutical composition” refers to an active agent in combination with a pharmaceutically acceptable carrier e.g. a carrier commonly used in the pharmaceutical industry. The phrase “pharmaceutically acceptable” is employed herein to refer to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment of any of the aspects, a composition as described herein further comprises at least one pharmaceutically acceptable carrier. Pharmaceutically acceptable carriers are well known in the art and include aqueous solutions such as physiologically buffered saline or other solvents or vehicles such as glycols, glycerol, vegetable oils (e.g., olive oil) or injectable organic esters. A pharmaceutically acceptable carrier can be used to administer a composition as described herein to a cell in vitro or to a subject in vivo. A pharmaceutically acceptable carrier can contain a physiologically acceptable compound that acts, for example, to stabilize the composition or to increase the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the vector described herein.

To facilitate delivery of a vector as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to animal and human subjects.

Prior to administration, the vectors described herein can be suspended in any pharmaceutically acceptable solution including sterile isotonic saline, water, phosphate buffered saline, 1,2-propylene glycol, polyglycols mixed with water, Ringer’s solution, etc. The exact number of viruses to be administered is not crucial success of the methods described herein, but should be an “effective amount,” i.e., an amount sufficient to drive TGF-β expression in the targeted cell type, e.g., cones, microglia or other neuronal or neuron-associated cells at a level that promotes neuronal cell survival and/or slows or halts neurodegeneration. In general, it is expected that the number of viruses (PFU) initially administered will be between 1 × 10⁶ and 1 × 10¹².

Injectable compositions can be prepared in conventional forms, either as liquid solutions or suspensions, solid forms suitable for dissolution or suspension in liquid prior to injection, or as emulsions. Alternatively, one can administer compositions described herein in a local, rather than systemic manner, for example, in a depot or sustained-release formulation. Further, a vector can be delivered adhered to a surgically implantable matrix (e.g., as described in U.S. Pat. Publication No. US-2004-0013645-Al). Alternatively, engineered vectors as disclosed herein can be in powder form (e.g., lyophilized) for constitution with a suitable vehicle, for example, sterile pyrogen-free water, before use.

Formulations for ophthalmic delivery can be used to deliver a vector directing the expression of a TGF-P polypeptide. Such formulations can generally comprise an admixture of the vector with an ophthalmically acceptable vehicle. An “ophthalmically acceptable vehicle” is one having physical properties (e.g., pH and/or osmolality) that are physiologically compatible with ophthalmic tissues, e.g., the retina, among others.

In some embodiments of any of the aspects, an ophthalmic composition is formulated as a sterile aqueous solution having an osmolality of from about 200 to about 400 milliosmoles/kilogram water (“mOsm/kg”) and a physiologically compatible pH. The osmolality of the solutions can be adjusted, for example, by means of conventional agents, such as inorganic salts (e.g., NaCl), organic salts (e.g., sodium citrate), polyhydric alcohols (e.g., propylene glycol or sorbitol) or combinations thereof.

Ophthalmic formulations can be in the form of liquid, solid or semisolid dosage form. Ophthalmic formulations can comprise, depending on the final dosage form, suitable ophthalmically acceptable excipients. In some embodiments, ophthalmic formulations are formulated to maintain a physiologically tolerable pH range. In certain embodiments, the pH range of an ophthalmic formulation is in the range of from about 5 to about 9. In some embodiments, pH of an ophthalmic formulation is in the range of from about 6 to about 8, or is about 6.5, about 7, or about 7.5. One or more ophthalmically acceptable pH adjusting agents and/or buffering agents can be included in a composition for ophthalmic delivery, including acids such as acetic, boric, citric, lactic, phosphoric, and hydrochloric acids; bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

In some embodiments of any of the aspects, a composition for ophthalmic delivery can be for topical delivery, e.g., in the form of an eye drop. By means of a suitable dispenser, a desired dosage of the active agent can be metered by administration of a known number of drops into the eye, such as by one, two, three, four, or five drops. Additional ocular pharmaceutical compositions and delivery devices are further described, e.g., in U.S. Pat. Nos. 9,993,558 B2; 4,310,543A; 8,668,676 B2, and 4,853, 224 A, the contents of each of which are incorporated herein by reference in their entireties.

### Administration, Dosing, and Efficacy

In one aspect, described herein are methods for treating or ameliorating a neurodegenerative disease or an ocular disease, event, or injury comprising administering the engineered vector or pharmaceutical composition described herein to a subject in need thereof. The compositions described herein can be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation.

The compositions described herein can be administered by any appropriate route which results in an increase in TGFβ receptor signaling, and/or maintenance of function or reduction in the destruction of neuronal tissue or cells (e.g., cone cells) in the subject. Depending upon the specific indication, the administering can be done by direct injection (e.g., directly administered to a target cell or tissue), subcutaneous injection, intramuscular injection, or topical delivery, or a combination thereof to the subject in need thereof. Additional exemplary modes of administration include ocular, intraocular, parenteral (e.g., intravenous, subcutaneous, intradermal, intramuscular, intradermal, intrapleural, intracerebral, topical (e.g., to the eye), intralymphatic, rectal, transmucosal, intranasal, inhalation (e.g., via an aerosol), buccal (e.g., sublingual), vaginal, intrathecal, transdermal, in utero (or in ovo), and the like. The most suitable route in any given case will depend on the nature and severity of the condition being treated and/or prevented and on the nature of the particular vector and/or formulation that is being used.

Dosages of engineered vectors and vector-containing pharmaceutical compositions to be administered to a subject depend upon the mode of administration, the disease or condition to be treated and/or prevented, the individual subject’s condition, the particular vector, the nucleic acid to be delivered, and the like, and can be determined in a routine manner. Exemplary doses for achieving therapeutic effects are viral titers of at least about 1 × 10⁴ infectious units, at least about 1 × 10⁵ infectious units, at least about 1 × 10⁶ infectious units, at least about 1 × 10⁷ infectious units, at least about 1 × 10⁸ infectious units, at least about 1 × 10⁹ infectious units, at least about 1 × 10¹⁰ infectious units, at least about 1 × 10¹¹ infectious units, at least about 1 × 10¹², or at least about 1 × 10¹³ infectious units introduced to the subject or the subject’s cells or tissues.

In some embodiments of any of the aspects, the subject is administered at least about 1 × 10¹¹ infectious units of the engineered vector described herein.

In some embodiments, more than one administration (e.g., two, three, four or more administrations) can be employed to achieve the desired level of gene expression over a period of various intervals, e.g., daily, weekly, monthly, yearly, etc.

An “effective amount” as used herein refers to the amount of engineered vector or pharmaceutical composition thereof needed to treat or alleviate a neurodegenerative or ocular disease. As a non-limiting example, where the disease is or includes an ocular neurodegenerative condition, alleviating the disease can be facilitated and the clinical outcome for the subject can be improved, including maintenance of function, or reduced risk for blindness or loss of visual acuity. For other neurodegenerative conditions, an effective amount provides a stabilization or reduction in loss of function such as memory, motor function or other neurological function. By “alleviate” in this context is meant a reduction in a symptom of the disease by at least 10% relative to the symptom occurring or expected to occur without administration of a composition as described herein. For example, loss, maintenance or improvement in visual acuity can be evaluated as described in the working examples herein (e.g., optomotor assay) or as known in the art, e.g., a Snellen eye chart, light perception tests, and motion tests. Visual acuity without an engineered vector as described herein can be about 90% or less compared with a healthy individual. The loss of function can be slowed, e.g., by at least 10% or more, by treatment with an effective amount of a composition as described. Alternatively, loss of function can be halted, and it is contemplated that in some instances, an improvement in function can also be effected. It is understood that for any given case, an appropriate “effective amount” can be determined by one of ordinary skill in the art using only routine experimentation.

In some embodiments of any of the aspects, the administration of an engineered vector as described herein can increase visual acuity in a subject with an ocular disease by at least 10% or more, 20% or more, 30% or more, 40% or more, 50% or more, 60% or more relative to a subject that has not received the engineered vector described herein. In some embodiments of any of the aspects, the administration of an engineered vector as described herein can increase visual acuity in a subject with an ocular disease by at least 10% or more, relative to acuity prior to administration.

With respect to other neurodegenerative disorders, e.g., Parkinson’s disease or multiple sclerosis, neurodegeneration and efficacy of treatment can be monitored using, for example, standard imaging and functional/behavioral tests known in the art. For example, motor control can be calculated as known in the art, e.g., tremor test, rigidity test, reflex tests, retropulsion tests, etc. Motor control without an engineered vector described herein can be about 90% or less compared to a healthy individual. Depending upon the disease and the disease status of the individual, loss of motor function can be slowed, e.g., by at least 10% by an effective treatment, or halted, or function can even be improved, e.g., by at least 10%, with the administration of an engineered vector as described herein. Halting loss of function can also be considered stabilization. As another example, treatment can be monitored for a subject with multiple sclerosis using the MS Disability Status Scale (DSS) or the Expanded Disability Status Scale (EDSS) metrics. The calculation of these measurements are described in detail, e.g., in Kurtzke JF. “Rating neurologic impairment in multiple sclerosis: an expanded disability status scale (EDSS).” Neurology. 1983 Nov;33(11):1444-52. doi: 10.1212/wnl.33.11.1444. PMID: 6685237; and Kurtzke JF. “Historical and clinical perspectives of the expanded disability status scale.” Neuroepidemiology. 2008;31(1):1-9. doi: 10.1159/0.0136645. Epub 2008 Jun 6. PMID: 18535394., the contents of which are incorporated herein by reference in their entireties. A slowing in DSS score deterioration, halting or stabilization of deterioration, or an improvement in DSS score, e.g., by 10% or one or more numerical grades, is evidence of effective treatment.

The effective dose of the compositions described herein can be estimated initially from cell culture assays, and a dose range can be formulated in animals (e.g., rodents). Data obtained from cell culture assays and animal studies can be used in formulating a range of dosage for use in humans. The dosage may vary within this range depending upon the dosage form employed and the route of use or administration utilized.

In some embodiments of any of the aspects, the administration of the engineered vector or pharmaceutical composition thereof is a single direct injection. In some embodiments of any of the aspects, the administration is continuous or repeated administration. In some embodiments, the administration is topical administration and/or an ocular injection including, for example, subretinal injection, retrobulbar injection, submacular injection, intravitreal injection, or intrachoroidal injection. In some embodiments of any of the aspects, the administering is subretinal injection.

[0.01] The compositions described herein can be used in combination with therapeutic agents for treating a neurodegenerative or ocular disease. Administered “in combination,” as used herein, means that two (or more) different treatments are delivered to the subject during the course of the subject’s affliction with the disorder, e.g., the two or more treatments are delivered after the subject has been diagnosed with the disorder (a neurodegenerative disease or an ocular disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The compositions described herein and/or at least one additional therapy can be administered simultaneously, in the same or in separate compositions, or sequentially.

Non-limiting examples of therapeutics for the treatment of neurodegenerative and ocular diseases include: anti-inflammatory medications, steroids, azathioprine, fingolimod, interferon 1β, glatiramer, natlizumab, Razadyne® (galantamine), Exelon® (rivastigmine), Aricept® (donepezil), rotigotine, carbidopa/levidopa, entacapone, ropinirole, cabergoline, pramipexole, tolcapone, bromocriptine, amantidine, benzotropine, vitamin A, lutein, omega-3 fatty acids, and Lucentis® (ranibizumab).

When administered in combination, the engineered vector or pharmaceutical composition thereof and the additional therapeutic agent can be administered in an amount or dose that is higher, lower, or the same as the amount or dosage of each composition individually, e.g., as a monotherapy.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen.

Dosing can be single dosage or cumulative (serial dosing), and can be readily determined by one skilled in the art. For instance, treatment of a disease or disorder may comprise a one-time administration of an effective dose of a vector or pharmaceutical composition disclosed herein. Alternatively, treatment of a disease or disorder can comprise multiple administrations of an effective dose of a vector carried out over a range of time periods, such as, e.g., once daily, twice daily, three times daily, once every few days, once weekly, once monthly, every two months, every three months, twice yearly, etc.

The timing of administration can vary from individual to individual, depending upon such factors as the severity of an individual’s symptoms. For example, an effective dose of a vector disclosed herein can be administered to an individual once every six months for an indefinite period of time, or until the individual no longer requires therapy. A person of ordinary skill in the art will recognize that the condition of the individual can be monitored throughout the course of treatment and that the effective amount of a vector as disclosed herein that is administered can be adjusted accordingly.

The effectiveness of a dosage, as well as the effectiveness of the overall treatment can be assessed by monitoring neuronal/eye structure and function using standard imaging and medical evaluation techniques known in the art. For example, improved or stable visual acuity is an indication that the treatment has been successful. If this does not occur or continue, repeat administration can be considered. Similarly, with non-ocular neurodegenerative diseases, progressive loss of cognition or motor function is an indication to consider or administer repeat dosing of a vector or pharmaceutical composition as described herein. It is contemplated that repeat dosing may include administration of a variant of the originally administered vector, e.g., to express a different TGF-β isoform, or to express it from a different promoter or regulatory elements.

A “therapeutically effective amount” is intended to mean the amount of vector or pharmaceutical composition or formulation comprising a vector which promotes neuronal and/or cone cell survival, providing attenuation or inhibition of neuronal cell loss or degeneration. An effective amount will vary, depending upon the pathology or condition to be treated, by the patient and his or her status, and other factors well known to those of skill in the art. Effective amounts are readily determined by those of skill in the art. Cell survival can be determined by methods known in the art, e.g., cell proliferation assays, retinal scans, light-dark discrimination assays, optomotor assays, vision tests, motor function tests, and cognition tests. For example, assays for neuronal cell viability are available commercially, e.g., the Invitrogen™ Neurite Outgrowth Staining Kit (Catalog # A15001). Additional methods are described, e.g, in Giordano G, Hong S, Faustman EM, Costa LG. Measurements of cell death in neuronal and glial cells. Methods Mol Biol. 2011;758:171-8. doi: 10.1007/978-1-61779-170-3_11. PMID: 21815065, the contents of which are incorporated herein by reference in its entirety.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

In some embodiments, the present application may be defined in any of the following, paragraphs:

1. An engineered vector comprising:

a retina-specific promoter operably linked to a nucleic acid sequence encoding a transforming growth factor beta (TGF-β) polypeptide.

2. The engineered vector of paragraph 1, wherein the engineered vector is selected from the group consisting of: an adeno-associated virus (AAV) vector; an adenovirus vector; and a lentiviral vector.

3. The engineered vector of paragraph 2, wherein the AAV vector is selected from the group consisting of: serotype AAV8; AAV2; AAV5; and AAV2/8.

4. The engineered vector of any one of paragraphs 1-3, further comprising a regulatory element.

5. The engineered vector of paragraph 4, wherein the regulatory element is Woodchuck Hepatitis Virus (WHV) Posttranscriptional Regulatory Element (WPRE).

6. The engineered vector of any one of paragraphs 1-5, wherein the TGF-β polypeptide is a TGF-β1 or TGF-β3 polypeptide.

7. The engineered vector of any one of paragraphs 1-6, wherein the retina-specific promoter is a red opsin promoter.

8. A pharmaceutical composition for the treatment of an ocular disease, the composition comprising:
(a) the engineered vector of any one of paragraphs 1-7; and
(b) a pharmaceutically acceptable carrier.

9. The pharmaceutical composition of paragraph 8, wherein the pharmaceutical composition is formulated for delivery to the eye.

10. The pharmaceutical composition of paragraph 8 or paragraph 9, wherein the pharmaceutical composition is formulated for delivery to the retina.

11. The pharmaceutical composition of paragraph 8 or paragraph 9, wherein the pharmaceutical composition is formulated as an eye drop.

12. The pharmaceutical composition of any of paragraphs 8-11, wherein the pharmaceutically acceptable carrier is an ophthalmically acceptable vehicle.

13. A method of treating an ocular disease in a subject, the method comprising: administering to the subject the engineered vector of any one of paragraphs 1-7 or the pharmaceutical composition of any one of paragraphs 8-12.

14. The method of paragraph 13, wherein the ocular disease is a neurodegenerative ocular disease.

15. The method of any one of paragraphs 13-14, wherein the ocular disease is selected from the group consisting of: retinitis pigmentosa; glaucoma; age-related macular degeneration; retinitis; sclerotic retinal maculodystrophy; diabetic retinopathy; proliferative retinopathy; toxic retinopathy; and retinopathy of prematurity.

16. The method of any one of paragraphs 13-15, wherein the administering is selected from the group consisting of: intraocular injection, subretinal injection, retrobulbar injection, submacular injection, intravitreal injection, intrachoroidal injection, topical application, eye drops, and intraocular implantation.

17. The method of any one of paragraphs 13-16, wherein the subject is a mammal.

18. The method of any one of paragraphs 13-17, wherein the subject is a human.

19. A method of promoting cone survival in the retina of a subject, the method comprising: intraocularly administering to the subject an effective amount of a composition comprising a vector comprising a nucleic acid construct comprising a retina-specific promoter operably linked to nucleic acid sequence encoding a transforming growth factor beta (TGF-β) polypeptide.

20. The method of paragraph 19, wherein the vector is an adenovirus vector, an AAV vector or a lentiviral vector.

21. The method of paragraph 19, wherein the TGF-β polypeptide is a TGF-β1 or TGF-β3 polypeptide.

22. The method of paragraph 20 or 21, wherein the vector is an AAV vector selected from the group consisting of: serotype AAV8; AAV2; AAV5; and AAV2/8.

23. The method of any one of paragraphs 19-22, wherein the retina-specific promoter is a red opsin promoter.

24. The method of any one of paragraphs 19-23, wherein the administering is selected from the group consisting of: intraocular injection, subretinal injection, retrobulbar injection, submacular injection, intravitreal injection, intrachoroidal injection, and intraocular implantation.

25. The method of any one of paragraphs 19-24, wherein the subject has or is suspected of having a neurodegenerative ocular disease.

26. The method of any one of paragraphs 19-25, wherein the subject is a mammal.

27. The method of any one of paragraphs 19-26, wherein the subject is a human.

28. The method of any one of paragraphs 25-27, wherein the ocular disease is selected from the group consisting of: retinitis pigmentosa; glaucoma; age-related macular degeneration; retinitis; sclerotic retinal maculodystrophy; diabetic retinopathy; proliferative retinopathy; toxic retinopathy; and retinopathy of prematurity.

29. A method of promoting neuronal cell survival, the method comprising: delivering a TGF-β polypeptide to a microglial cell.

30. The method of paragraph 29, wherein the TGF-β polypeptide is a TGF-β1 or TGF-β3 polypeptide.

31. The method of paragraph 29 or 30, wherein the delivering comprises administering a vector encoding the TGF-β polypeptide to a neuronal cell associated with the microglial cell.

32. The method of paragraph 31, wherein the vector is selected from the group consisting of: an adeno-associated virus (AAV) vector; an adenovirus vector; and a lentiviral vector.

33. The method of paragraph 32, wherein the vector is an AAV vector selected from the group consisting of: serotype AAV8, AAV2, and AAV2/8.

34. The method of any one of paragraphs 31-33, wherein the vector comprises a promoter active in a neuronal cell, operatively linked to nucleic acid sequence encoding the TGF-β polypeptide.

35. The method of paragraph 34, wherein the promoter is a retina-specific promoter.

36. The method of paragraph 35, wherein the promoter is active in a cone cell or a microglial cell.

37. The method of any one of paragraphs 34-36, wherein the promoter is a red opsin promoter.

38. The method of any one of paragraphs 29-37, wherein the delivering promotes signaling through a TGFBR1 and/or TGFBR2 receptor.

39. A method of treating a neurodegenerative disease or disorder in a subject in need thereof, the method comprising: administering to the subject a viral vector comprising a promoter active in a neuronal cell operatively linked to a nucleic acid sequence encoding a TGF-β polypeptide.

40. The method of paragraph 39, wherein the TGF-β polypeptide is a TGF-β1 or TGF-β3 polypeptide.

41. The method of paragraph 39, wherein the viral vector is an AAV vector.

42. The method of paragraph 41, wherein the AAV vector is selected from the group consisting of: serotype AAV8; AAV2; AAV5; and AAV2/8.

43. The method of any one of paragraphs 39-42, wherein the viral vector comprises a retina-specific promoter

44. The method of paragraph 43, wherein the retina-specific promoter is a red opsin promoter.

45. The method of any one of paragraphs 39-44, wherein the administering is selected from the group consisting of: systemic injection, direct injection, intraocular injection, subretinal injection, retrobulbar injection, submacular injection, intravitreal injection, intrachoroidal injection, and intraocular implantation.

46. The method of any one of paragraphs 39-45, wherein the neurodegenerative disease or disorder is selected from the group consisting of: retinitis pigmentosa; glaucoma; macular degeneration; retinitis; retinal maculodystrophy; diabetic retinopathy; Alzheimer’s disease, Parkinson’s disease, Huntington’s disease, amyotrophic lateral sclerosis (ALS), frontotemporal dementia, chronic traumatic encephalopathy (CTE), multiple sclerosis, and neuroinflammation.

47. The method of any one of paragraphs 39-46, wherein the subject is a mammal.

48. The method of any one of paragraphs 39-47, wherein the subject is a human.

49. Use of the engineered vector of any of paragraphs 1-7 for the treatment of an ocular disease.

50. Use of the engineered vector of any of paragraphs 1-7 for the treatment of retinitis pigmentosa.

### EXAMPLES

The methods and compositions provided herein are for use in the treatment of an ocular disease, e.g., a degenerative eye disease such as retinitis pigmentosa. The compositions and methods provided herein are based, in part, on the discovery that a novel agent that modulates TGF-β1 (e.g., AAV-8-TGFB1) provides protection of cones in the eye and restores vision in animal models of retinitis pigmentosa.

### Example 1: Modulation of Microglia by TGF-β1 as a Generic Therapy for Retinitis Pigmentosa

Retinitis pigmentosa (RP) is a genetically heterogeneous group of eye diseases in which initial degeneration of rods triggers secondary degeneration of cones, leading to significant loss of daylight, color, and high-acuity vision. Gene complementation with adeno-associated viral (AAV) vectors is one strategy to treat RP. Its implementation faces substantial challenges, however - e.g., the tremendous number of loci with causal mutations. Gene therapy targeting secondary cone degeneration is an alternative approach that could provide a much-needed generic treatment for many RP patients. Here, we show that microglia are required for the upregulation of potentially neurotoxic inflammatory factors during cone degeneration in RP, creating conditions that might contribute to cone dysfunction and death. To ameliorate the effects of such factors, we used AAV vectors to express isoforms of the anti-inflammatory cytokine transforming growth factor-beta (TGF-β). AAV-mediated delivery of TGF-β1 rescued degenerating cones in three mouse models of RP carrying different pathogenic mutations. Treatment with TGF-β1 protected vision, as measured by two behavioral assays, and could be pharmacologically disrupted by either depleting microglia or blocking the TGF-β receptors. The results indicate that TGF-β1 can be broadly beneficial for patients with cone degeneration, and potentially other forms of neurodegeneration, through a pathway dependent upon microglia.

### Introduction

Retinitis pigmentosa (RP) is a genetically heterogeneous group of eye diseases that causes progressive loss of vision due to the dysfunction and degeneration of photoreceptors. Globally, the condition affects an estimated two million people, with thousands of pathogenic mutations identified to date spanning at least 80 different genes (1). In RP, there is early death of rods, the photoreceptors needed for vision in dim light, leading to difficulty with night vision typically by adolescence (2, 3). Rod degeneration is then followed by the dysfunction and death of cones, the cells essential for daylight, color, and high-acuity vision, loss of which can eventually result in blindness (3, 4). The pathogenesis of cone degeneration in RP is not understood, in part due to the fact that causal mutations are often exclusively expressed in rods, suggesting that cone death may be driven by a set of converging mechanisms independent of the genetic lesion (4). Despite ongoing efforts to characterize these mechanisms, there are still no widely accepted interventions to halt primary rod degeneration or secondary cone degeneration in patients with RP (5, 6).

One proposed treatment for RP and other inherited retinal diseases (IRDs) is the use of gene therapy to introduce an allele that can complement the mutation. This strategy recently led to the first commercial gene therapy for an IRD and has tremendous therapeutic promise (7, 8). Nonetheless, its implementation for RP faces several key challenges. Specifically, developing a gene therapy and clinical trial for each disease gene in RP will be logistically difficult considering the large number of genes to target, but the limited number of individuals with any given mutation (1). Because RP may go undiagnosed until the onset of night blindness (3), patients might also not have sufficient rods for correction of the genetic lesion. In addition to these obstacles, RP due to autosomal dominant or unidentified mutations, which together comprise one-third of cases (9), is not amenable to gene complementation and thus requires an alternative approach. To address these challenges, we and others have focused instead on the development of gene therapy targeting secondary cone degeneration (10-12), the process ultimately responsible for loss of quality of life in RP. Such therapies, if successful, would provide a much-needed and broadly applicable treatment option for the many patients with RP for which gene therapy is otherwise infeasible.

Microglia are the resident immune cells of the retina and central nervous system (CNS). In response to infection or tissue damage, they can become activated, a state characterized by changes in microglial morphology, phagocytosis, and cytokine production (13, 14). Excessive microglial activation has been implicated in virtually every neurodegenerative disorder (13-15), including RP, in which activated microglia in the retina have been shown to phagocytose photoreceptors (16). During primary rod degeneration in RP, activated microglia appear to be harmful as ablating these cells or suppressing their activation have been reported to enhance rod survival (16, 17). However, how microglia contribute to secondary cone degeneration is less clear. In a previous study of cone degeneration in RP, soluble CX3CL1 (fractalkine) was overexpressed, a secreted molecule thought to regulate activation of microglia through a receptor on their surface (12). While soluble fractalkine prolonged cone survival and function in RP mouse models, it surprisingly did not require microglia to do so. In the current study, we further addressed the role of microglia in cone death by overexpressing different isoforms of transforming growth factor beta (TGF-(β), an anti-inflammatory cytokine known to inhibit microglial activation (18, 19). Using three mouse models of RP, we found that TGF-β1 was able to protect degenerating cones and save vision via a mechanism that required both microglia and TGF-β receptor signaling. Our data support the application of TGF-β1 as a generic therapy for patients with RP and highlight the therapeutic potential of modulating microglia to treat neurodegenerative conditions.

### Results

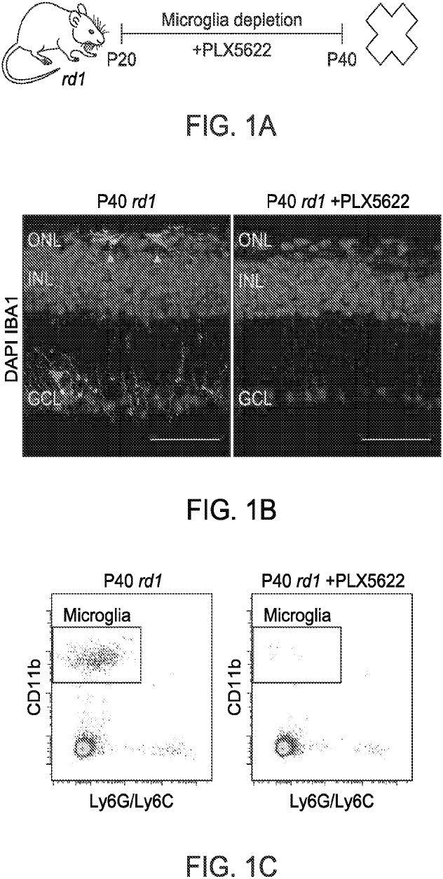

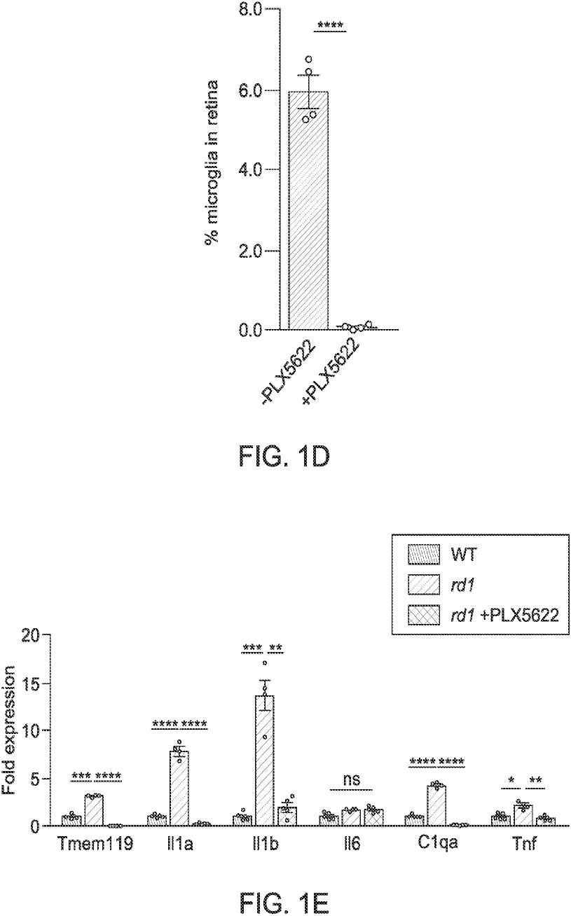

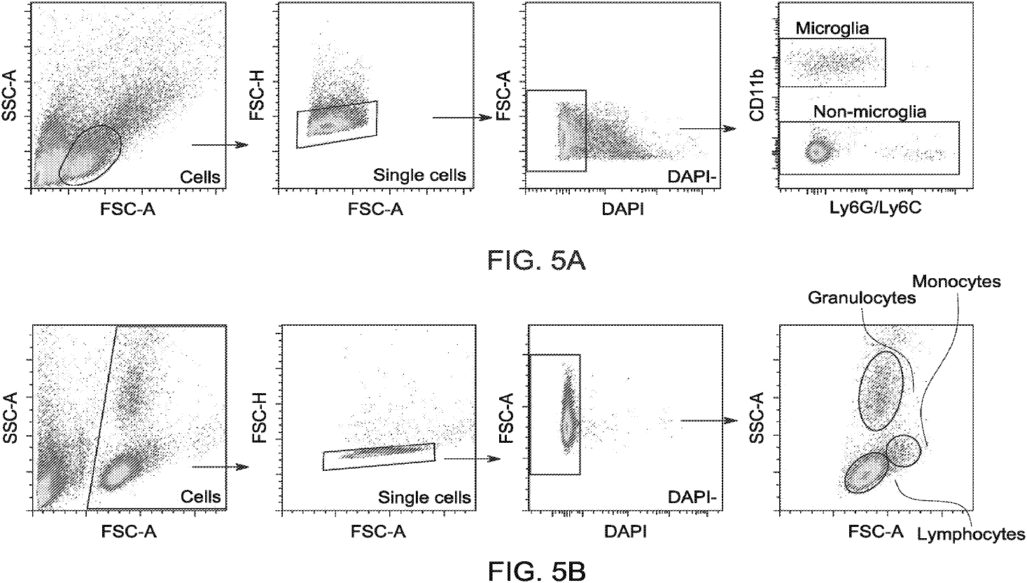

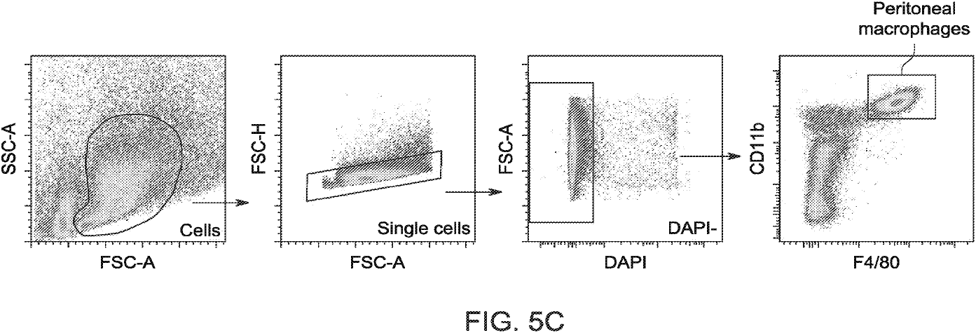

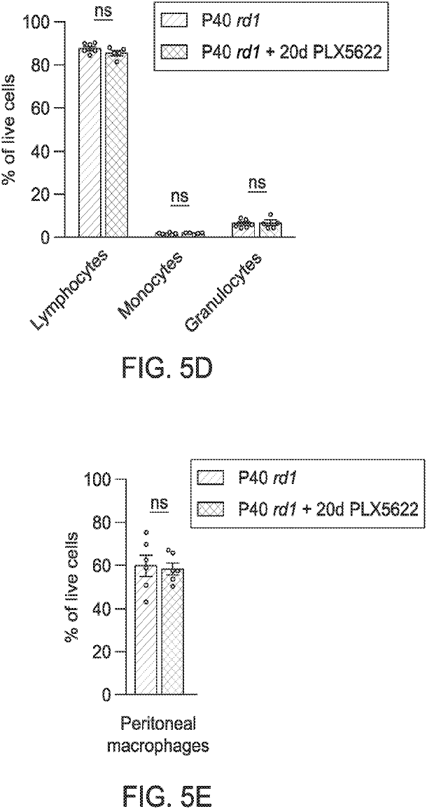

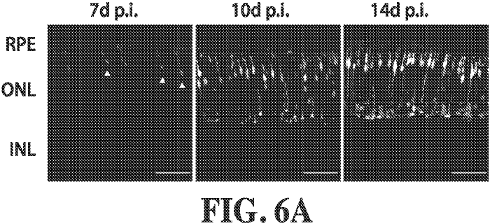

To examine the effects of microglia during secondary cone degeneration, mice were treated with PLX5622, a potent colony stimulating factor 1 receptor (CSF1R) inhibitor that eliminates microglia (20). In the rd1 mouse line, the most widely used animal model of RP (21), PLX5622 treatment for 20 days depleted ~99% of retinal microglia (FIGS. 1A-1D) but grossly preserved peripheral immune populations, such as circulating monocytes and peritoneal macrophages (FIGS. 5B-5E). We previously found that during secondary cone degeneration, there is persistent upregulation in the retina of Tmem119, a marker for microglia (22), as well as Il1a, Il1b, C1qa, and Tnf (12), inflammatory factors that have been shown to induce neurotoxicity both in vitro and in vivo (15, 23, 24). Here, we confirmed these findings (FIG. 1E) and sought to determine if microglia were not just correlated with, but responsible for the upregulation of inflammatory genes. RT-PCR performed on retinas from rd1 mice with or without PLX5622 treatment demonstrated that increased expression of Il1a, Il1b, C1qa, and Tnf was abolished following microglial depletion (FIG. 1E). These data strengthened our hypothesis that microglia play a causal role in retinal inflammation during secondary cone degeneration.

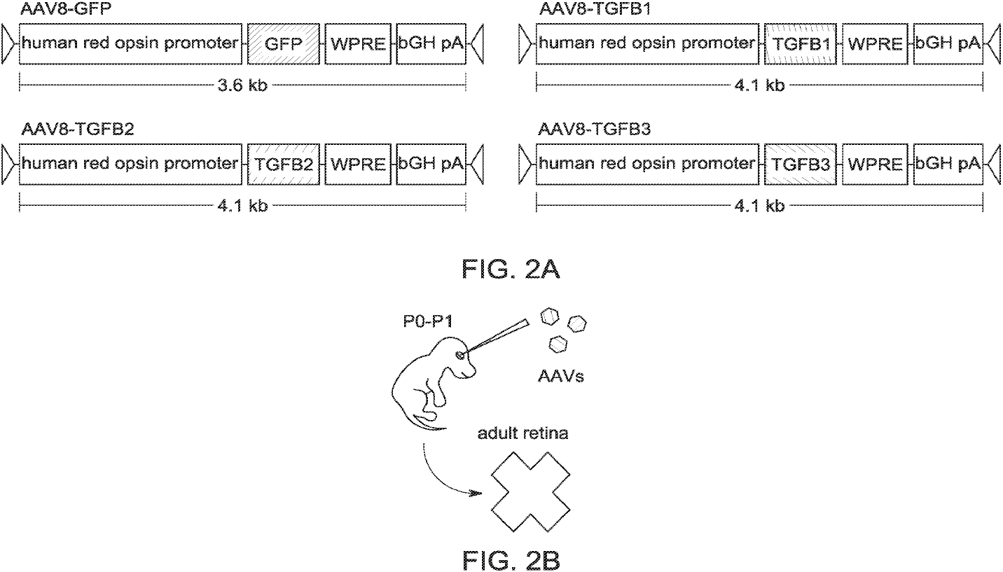

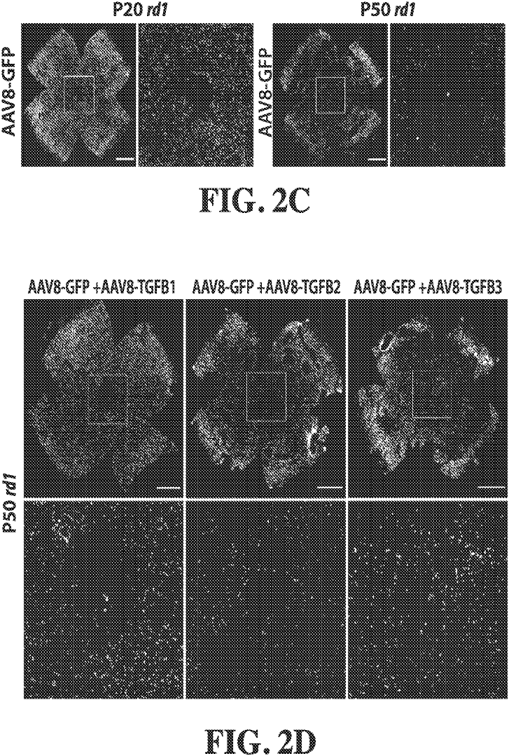

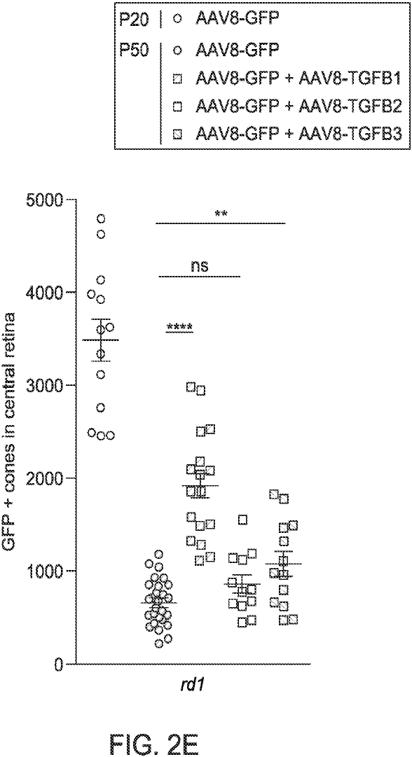

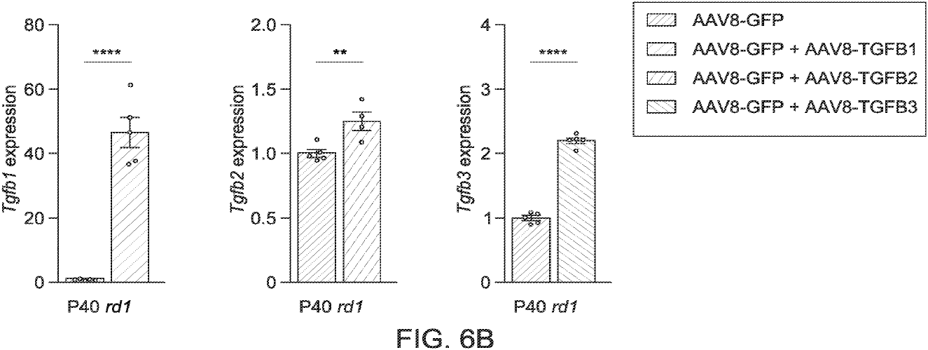

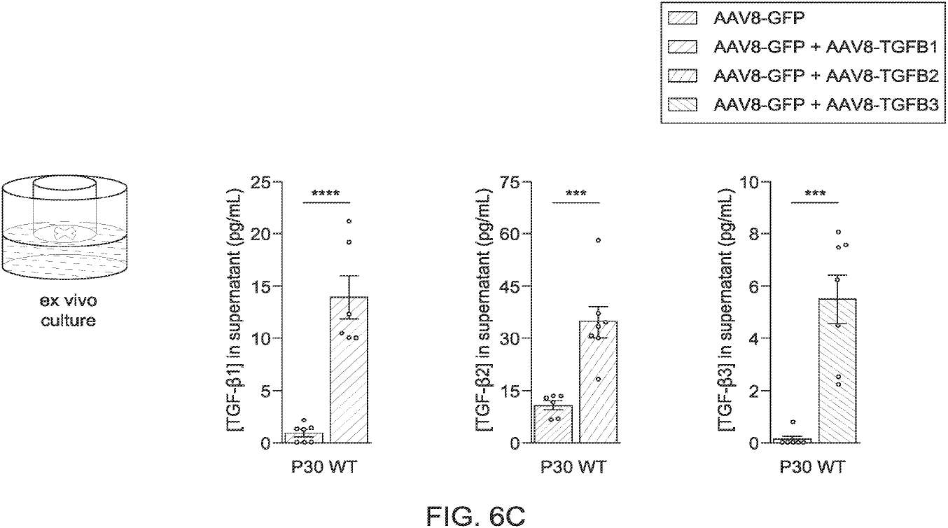

TGF-β is a major anti-inflammatory cytokine that signals through the TGF-β type I (TGFBR1) and II (TGFBR2) receptors to trigger downstream expression of target genes (25). Exogenous TGF-β can inhibit microglial production of inflammatory cytokines such as Tnf and Il6 (18, 19), whereas ablation of TGF-β signaling in microglia via genetic deletion of TGFBR2 leads to activation of these cells (26) and, notably, degenerative changes in the retina highly reminiscent of RP (27). We reasoned that suppressing microglial activation and its resulting inflammation with TGF-β might be beneficial for degenerating cones in RP. To test this idea, adeno-associated viral (AAV) vectors encoding each of the TGF-β isoforms - TGF-β1 (AAV8-TGFB1), TGF-β2 (AAV8-TGFB2), and TGF-β3 (AAV8-TGFB3) - were generated and subretinally injected into rd1 mice at postnatal day 0-1 (P0-P1), a time point enabling infection of photoreceptors throughout the entire retina (FIGS. 2, A and B). These vectors used the human red opsin promoter to drive expression in cones (28) and were co-administered with a previously described GFP vector (AAV8-GFP) employing the same promoter to facilitate cone quantification (11, 12). GFP driven by the human red opsin promoter could first be detected in cones around 7 days post-injection, with strong expression by day 14 (FIG. 6A). AAV vectors with this same promoter resulted in significant upregulation of TGF-β isoforms in infected retinas at both the mRNA and protein levels (FIGS. 6B and 6C).

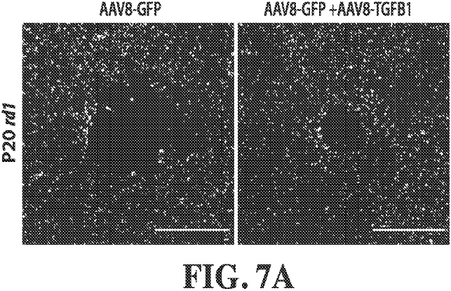

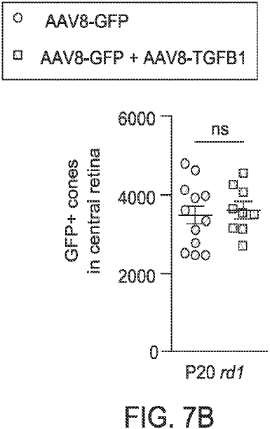

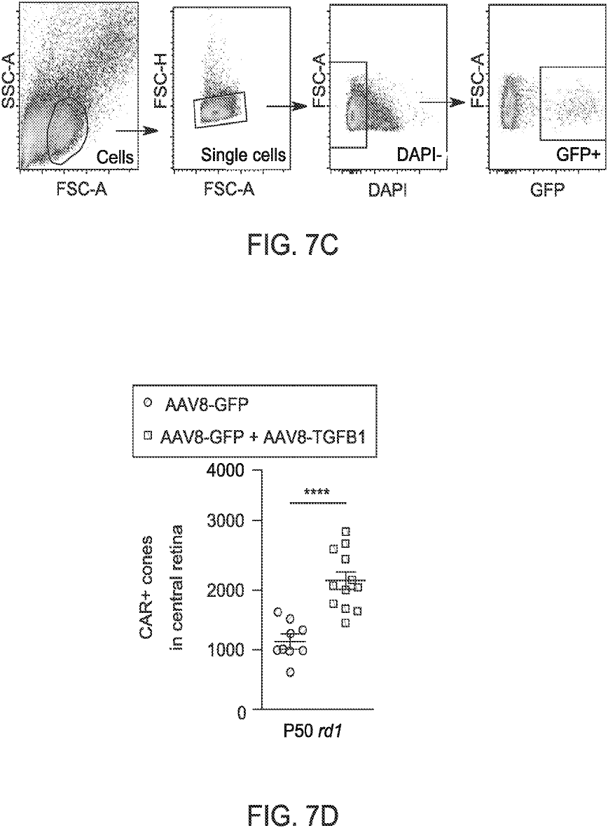

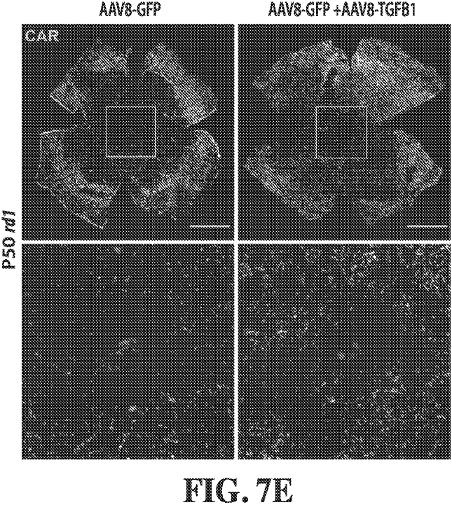

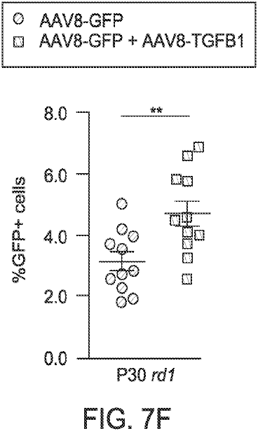

Secondary cone degeneration begins around P20 in rd1 mice, with massive loss of cones by P50, particularly within the central retina (FIG. 2C). To measure the effect of TGF-β isoforms on retinal degeneration, the number of GFP-positive cones in the central retina was therefore quantified. Compared to AAV8-GFP alone, there was no significant difference in the number of cones at P50 with the addition of AAV8-TGFB2, and only a modest increase with AAV8-TGFB3 (FIGS. 2D and 2E). In contrast, infection with AAV8-TGFB1 nearly tripled the number of cones in the central retina at P50. To determine whether greater cone numbers with TGF-β1 were a result of cone preservation or rather a perturbation in retinal development, rd1 retinas treated with AAV8-GFP or AAV8-GFP plus AAV8-TGFB1 were examined at P20, prior to secondary cone degeneration. AAV8-TGFB1 did not alter the number of cones at this time point (FIGS. 7A and 7B), suggesting that the difference in cones at P50 was indeed due to prolonged survival. As increased cone counts with TGF-β1 could also be explained by a rearrangement of peripheral cones to the central retina, whole rd1 retinas were analyzed at P30 by flow cytometry, which showed significantly more GFP-positive cones in eyes treated with AAV8-GFP plus AAV8-TGFB1 compared to AAV8-GFP only (FIGS. 7C and 7D). Finally, to verify that TGF-β1 was improving the survival of GFP-positive cones and not just upregulating GFP expression, rd1 retinas at P50 were immunostained for cone arrestin, a marker of all cones, which again demonstrated significantly more cones in the central retina with the addition of AAV8-TGFB1 (FIGS. 7E and 7F). Together, these data indicated that AAV8-TGFB1 could rescue degenerating cones in the rd1 model of RP.

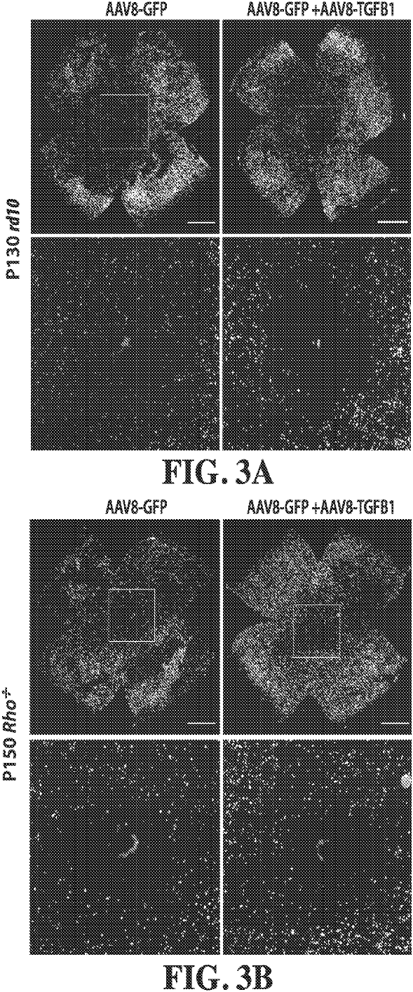

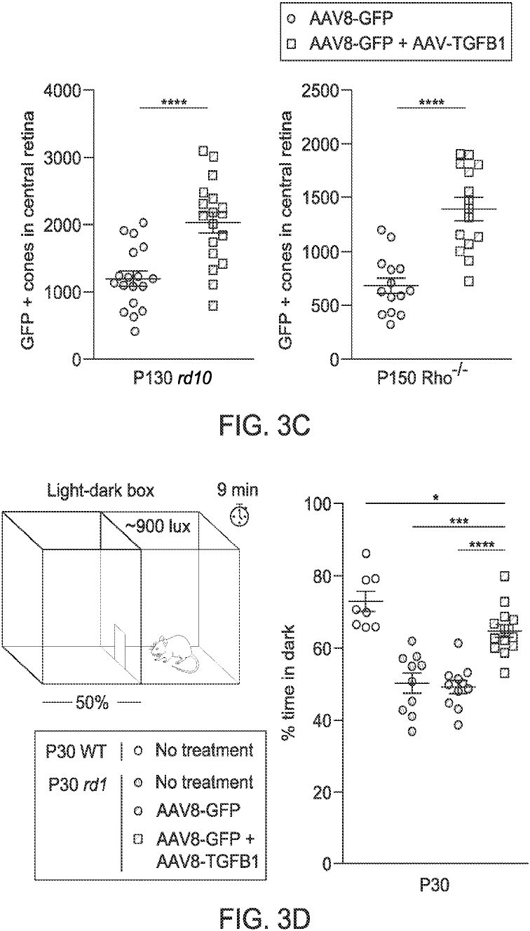

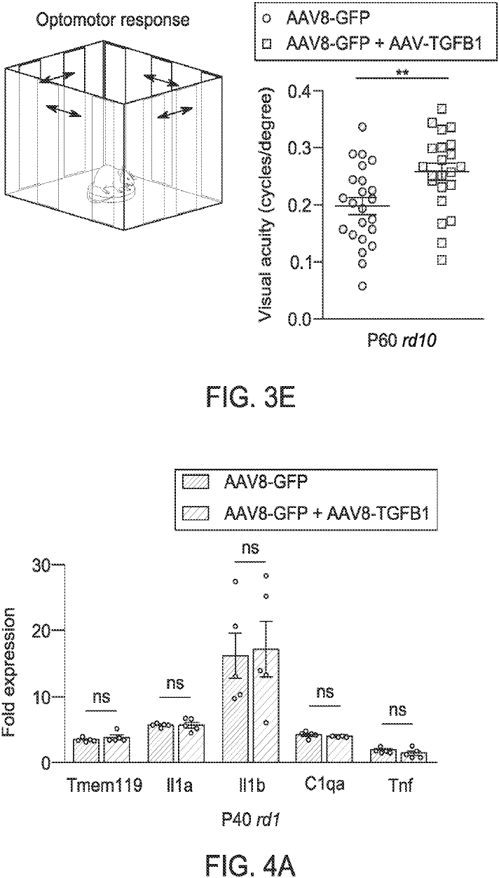

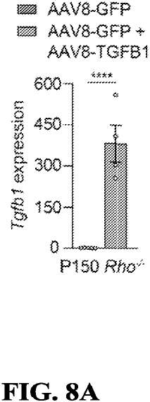

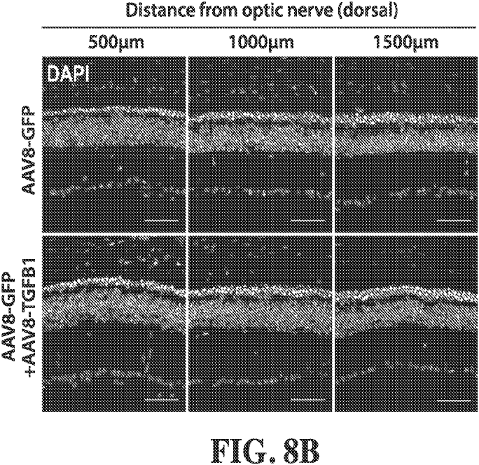

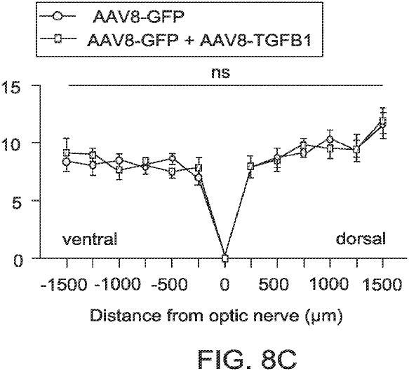

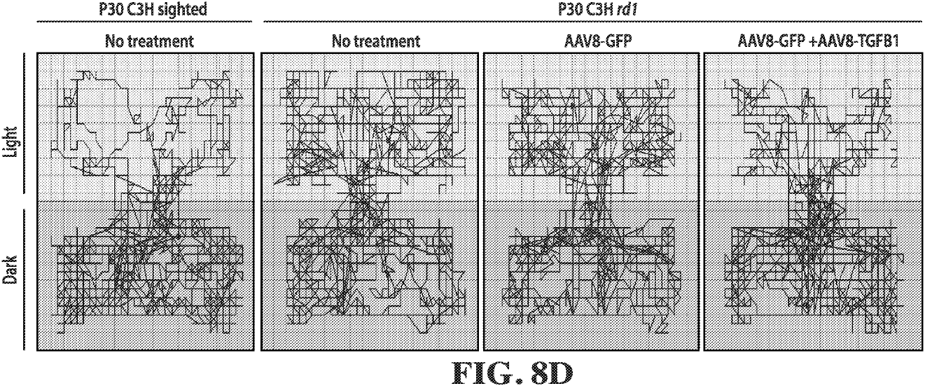

AAV8-TGFB1 was next studied in two more slowly degenerating mouse models of RP: rd10, which harbors a mutation in Pde6b, a common cause of autosomal recessive RP (21), and Rho-/-, which lacks rhodopsin, the most frequently mutated gene in autosomal dominant RP (29). Upregulation of Tgfb1 with AAV8-TGFB1 persisted in these older mice (FIG. 8A). In both strains, AAV8-TGFB1 again significantly improved cone survival (FIGS. 3A-3C), implying that TGF-β1 might be generically beneficial for cones in RP. The impact of TGF-β1 on rod survival was additionally investigated in rd10 mice by measuring the thickness of the outer nuclear layer (ONL), which normally consists primarily of rods. Despite preserving cones in the same model, AAV8-TGFB 1 did not prevent rod death and the reduction of ONL thickness in rd10 retinas (FIGS. 8B and 8C). Thus, the therapeutic effect of AAV8-TGFB1 in RP appears to be selective for cones.

Encouraged by our histological findings, we assessed the potential clinical relevance of TGF-β1 gene therapy by subjecting treated mice to a light-dark discrimination test. Sighted mice spend less time in well-illuminated spaces as demonstrated by the strong preference of wild-type animals for the dark half of a 50:50 light-dark box (FIG. 3D and FIG. 8D). Conversely, rd1 mice, which can no longer distinguish light from dark by P30 due to loss of functional photoreceptors, equally split their time between the two compartments. Compared to animals without treatment or receiving AAV8-GFP only, rd1 mice treated with AAV8-GFP plus AAV8-TGFB1 spent significantly more time in the dark, consistent with an improvement in visual function allowing for light-dark discrimination. As a complementary measure of vision, the optomotor assay was performed on rd10 mice treated with AAV8-GFP in one eye and AAV8-GFP plus AAV8-TGFB1 in the other. In this experiment, moving stripes are used to elicit the visually-dependent optomotor response. By adjusting the stripes until the animal can no longer track them, the visual acuity in each eye can be estimated (30). At P60, rd10 eyes treated with AAV8-GFP plus AAV8-TGFB1 exhibited significantly better visual acuity than those only receiving AAV8-GFP (FIG. 3E). From these data, we concluded that TGF-β1 in mouse RP not only helps preserve cones, but also importantly protects from vision loss.

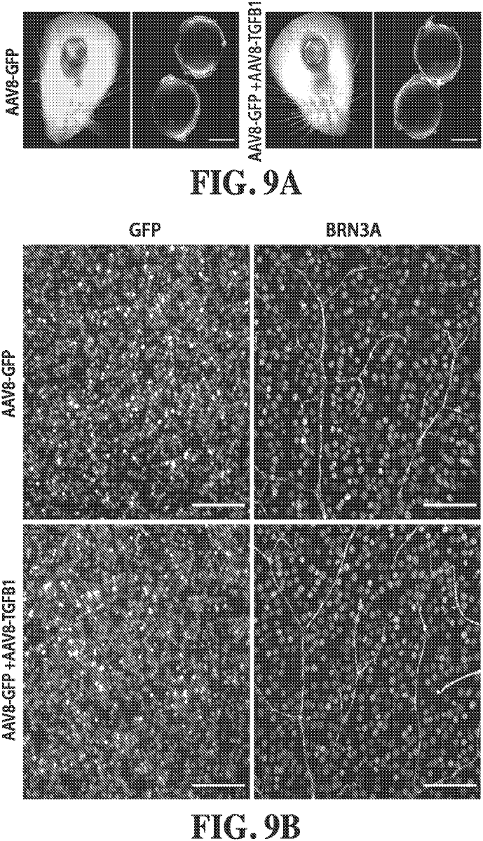

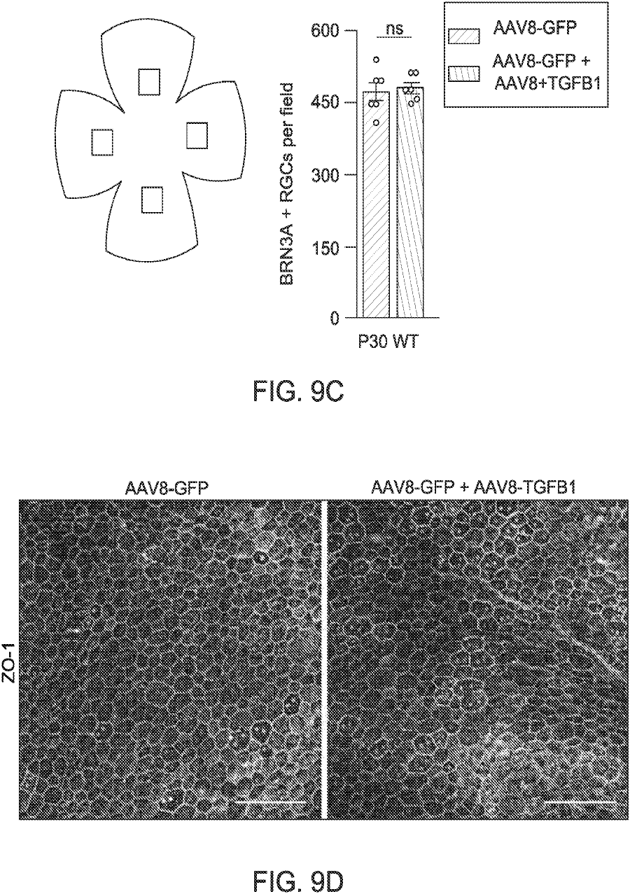

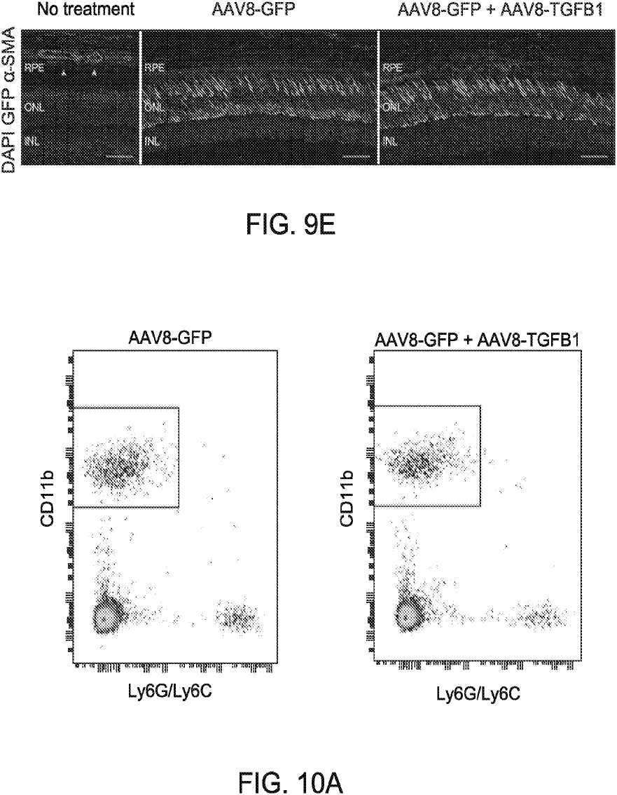

Although we found AAV8-TGFB1 to be beneficial for cones, TGF-β signaling in the eye has also been reported to mediate cataract formation (31, 32), ocular hypertension leading to loss of retinal ganglion cells (RGCs) (32), and epithelial-mesenchymal transition (EMT) in the retinal pigment epithelium (RPE), a process implicated in proliferative vitreoretinopathy (33, 34). Mice treated with AAV8-GFP or AAV8-GFP plus AAV8-TGFB1 were thus examined for these possibilities. At P30, no obvious difference in the opacity of the lens was seen in animals receiving AAV8-GFP plus AAV8-TGFB1 compared to AAV8-GFP only (FIG. 9A). Moreover, treatment with AAV8-TGFB1 did not impact the number of RGCs at this time point (FIGS. 9B and 9C). To assess for EMT in the RPE, immunostaining was performed for ZO-1, a component of epithelial tight junctions (35), and α-smooth muscle actin (α-SMA), which labels RPE cells undergoing EMT (33, 34). Neither of these proteins were qualitatively changed in the RPE with the addition of AAV8-TGFB1 (FIGS. 9D and 9E), suggesting that at least up to one month post-treatment, AAV8-TGFB1 is not noticeably disruptive in the eye.

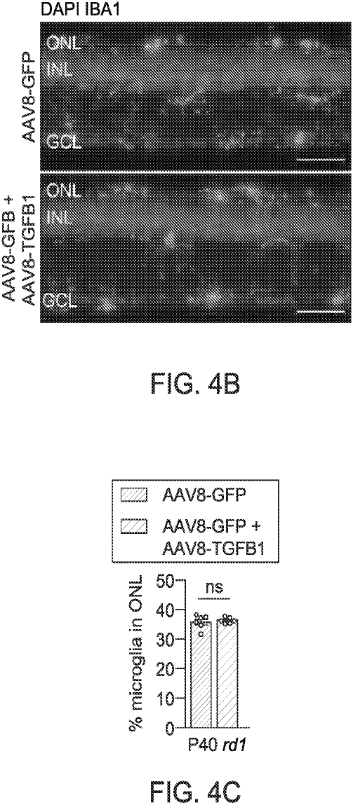

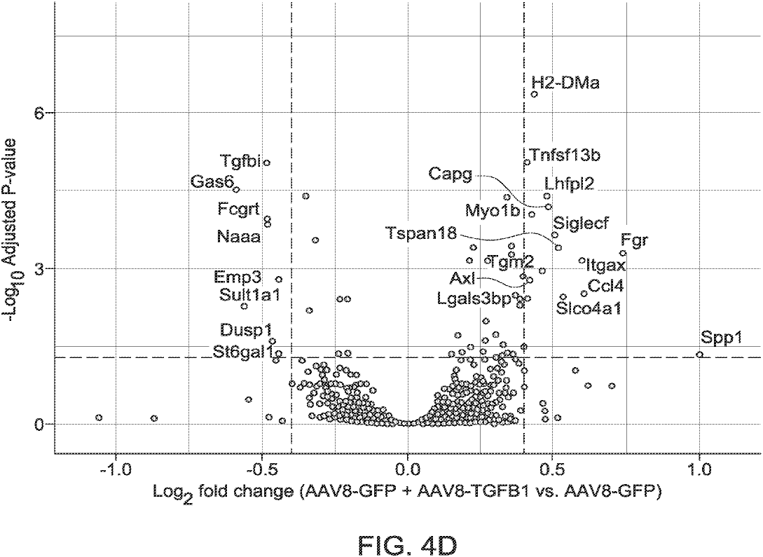

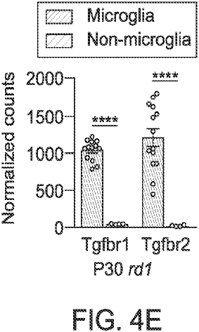

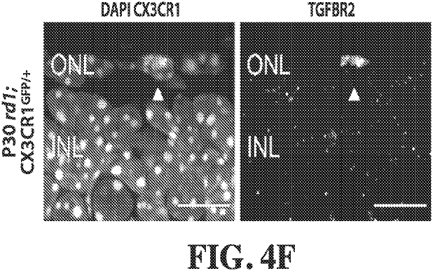

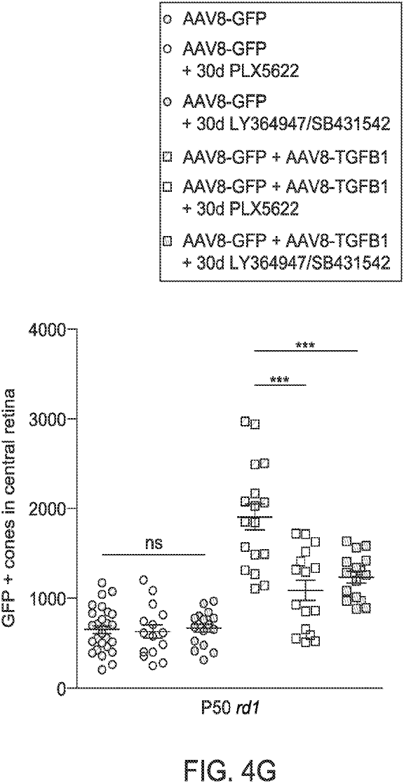

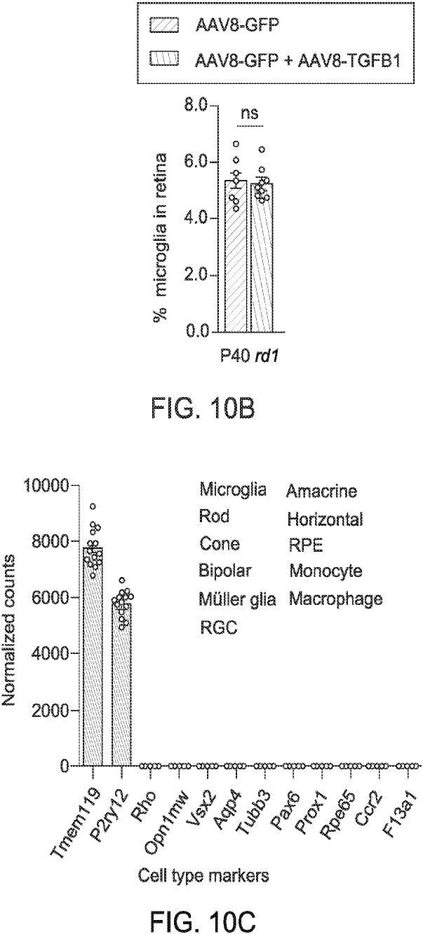

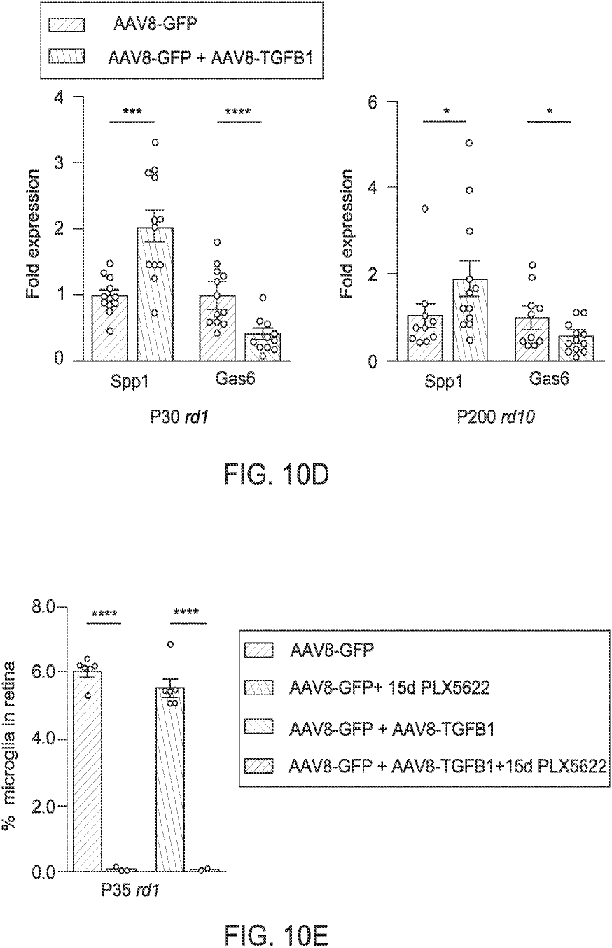

How does AAV8-TGFB1 combat secondary cone degeneration? Given the anti-inflammatory properties of TGF-β, mRNA levels of Tmem119, Il1a, Il1b, C1qa, and Tnf in P40 rd1 retinas were quantified and, surprisingly, were found to be unchanged with AAV8-TGFB1 (FIG. 4A). AAV8-TGFB1 likewise did not affect the number of microglia in the retina as assayed by flow cytometry (FIGS. 10A and 10B), and treatment did not alter the percentage of microglia in the ONL (FIGS. 4B and 4C), the retinal layer in which microglia preferentially localize during degeneration (12). To better understand the microglial response to AAV8-TGFB1, microglia from P30 rd1 retinas treated with AAV8-GFP or AAV8-GFP plus AAV8-TGFB1 were isolated by cell sorting and subjected to RNA sequencing (RNA-seq). Sorted microglia were highly pure, expressing microglia markers such as Tmem119 and P2ry12, but not those of other cell types (FIG. 10C). Only 23 genes were significantly altered (adjusted P<0.05, log2 fold change >0.4) in microglia treated with AAV8-TGFB1 (FIG. 4D). These included Spp1 and Gas6, the most upregulated and downregulated of the 23 genes, respectively, which were validated by RT-PCR in microglia from both P30 rd1 and P200 rd10 retinas (FIG. 10D).

The importance of these gene expression changes in microglia was subsequently evaluated by depleting microglia from mice treated with AAV8-TGFB1 during secondary cone degeneration. Beginning at P20, rd1 mice were administered PLX5622, which eliminated ~99% of retinal microglia even in eyes infected with AAV vectors (FIG. 10E). While microglial depletion had no significant effect on cone survival in rd1 retinas treated with AAV8-GFP only (FIG. 4G), consistent with our prior observations (12), it significantly abrogated cone rescue by AAV8-TGFB1. These findings indicate that microglia are not inherently helpful or harmful for degenerating cones, but are necessary for the cone survival mediated by TGF-β1 gene therapy. In the retina, microglia are among the only cells that highly express TGFBR1 and TGFBR2 (FIGS. 4, E and F) (27), both of which are required for TGF-β signaling (25). We therefore hypothesized that AAV8-TGFB1 might act via TGF-β receptors on microglia in order to promote cone survival. To test this, rd1 mice treated with AAV8-GFP or AAV8-GFP plus AAV8-TGFB1 were administered a combination of LY364947 and SB431542, potent TGFBR½ inhibitors capable of blocking these receptors in vivo (36). As with microglial depletion, TGFBR½ inhibition had no discernable effect on retinas treated with AAV8-GFP only (FIG. 4G), suggesting that any endogenous signaling through these receptors during cone degeneration did not dramatically affect cone survival. On the other hand, treatment with LY364947 and SB431542 significantly disrupted the ability of AAV8-TGFB1 to preserve cones (FIG. 4G). Collectively, these results demonstrate that both microglia and TGF-β signaling through TGFBR1 and TGFBR2 are needed for AAV8-TGFB1 to function therapeutically.

### Discussion

Virally introduced TGF-β, exemplified by AAV8-TGFB1, provides a novel gene therapy that protects cones and vision in multiple mouse models of RP, supporting its translation to different genetic forms of retinal degeneration in patients. Interestingly, although depletion of microglia itself does not help or hinder cone survival, cone rescue by AAV8-TGFB1 requires microglia. Together, these data indicate that microglia do not play a significantly negative role during cone degeneration in RP, but under certain conditions, can be induced to benefit cones. This study further shows a dependence of TGF-β gene therapy upon TGFBR1 and TGFBR2, which likely mediate signaling directly within microglia. While not wishing to be bound by theory, we favor a model in which this signaling induces microglia to create a retinal environment favorable to cone survival. These findings thus highlight a new immunomodulatory strategy centered around microglia for treating patients with RP, an approach that can also be relevant for other degenerative diseases of the visual system and CNS.

Of note, dependence of TGF-β1 gene therapy upon microglia in this study was determined using PLX5622, a CSF1R inhibitor which depleted up to ~99% of retinal microglia. However, CSF1R is likewise present on monocytes and other macrophages in the body, and although the majority of these populations are not depleted with PLX5622 (37), their functions could theoretically be affected by CSF1R inhibition. While possible contributions from monocytes or macrophages residing in the choroid cannot be excluded, retinal microglia are the most likely effector cells of TGF-β therapy via AAV8-TGFB1 given their high expression of the TGF-β receptors and proximity to degenerating cones. It should further be mentioned that in both microglial depletion and TGFBR½ inhibition experiments, cone rescue with AAV8-TGFB1 was not fully eliminated. While not wishing to be bound by theory, this could have been due to therapeutic activity from TGF-β1 prior to P20, as PLX5622, LY364947, and SB431542 were not administered until this age. Alternatively, blocking of the relevant receptors by these drugs may have been incomplete, leading to ablation of most, but not all, of the treatment effect.

While not directly relevant to the therapeutic efficacy of the methods described herein, one question that remains is how exactly microglia preserve cones in response to TGF-β1. Surprisingly, RNA-seq of retinal microglia from P30 rd1 mice only identified 23 genes that were significantly altered with AAV8-TGFB1. This list included Spp1, which is upregulated in microglia associated with RPE protection (38), but did not contain any genes already known to aid in cone survival. While not wishing to be bound by theory, in treated eyes, it is conceivable that microglia become less sensitive to elevated TGF-β1 levels over time, resulting in fewer and less pronounced transcriptional changes. RNA-seq of these microglia at a time point earlier than P30 may therefore uncover additional differences in gene expression that were not captured in these analysis. Alternatively, the therapeutic effects of AAV8-TGFB1 may occur via changes not detectable by RNA-seq, such as through post-translational modifications of the proteome.

Clinically, a major appeal of AAV-mediated gene therapy is the prospect of sustained or even lifelong treatment following a single dose of vector. Nonetheless, receiving a long-term treatment also carries risks, which must be carefully weighed against the benefits of therapy. With AAV8-TGFB1, of particular concern were the possibilities of cataract formation, RGC death, and EMT in the RPE, any of which could be detrimental to vision. Reassuringly, none of these complications were observed at one month after vector delivery, supporting the safety of TGF-β polypeptide gene therapy in the eye. Notable differences between the methodologies of this and prior studies may explain why AAV8-TGFB1 was found to be well tolerated. Using an adenoviral vector, Robertson et al. showed that overexpression of TGF-β1 in rats could cause fibrosis in the lens and severe RGC loss as early as two weeks post-injection (32). However, this vector was administered in the anterior segment of the eye rather than the subretinal space, employed a promoter with much broader expression, and, being an adenoviral vector, was substantially more inflammatory than the AAV vectors tested here (39). In the RPE, TGF-β1 has been widely used to study EMT in vitro as it initiates loss of epithelial markers and upregulates α-SMA in cultured RPE cells (33, 34). Even so, it is unclear whether this effect of TGF-β1 can be extended in vivo, as experiments conducted on sheets of RPE suggest that normal cell-cell contact, which is absent from cell culture models, is sufficient to prevent induction of EMT by TGF-β signaling (40).

Compared to mouse models of RP, which undergo cone degeneration over the span of months, humans with the disease typically begin losing their cones as young adults with progression over multiple decades (3, 41). Based on these kinetics, it is contemplated that prolonged cone survival for several months with AAV8-TGFB1 as demonstrated in this study can translate to years of meaningful vision for patients. With the addition of TGF-β1, there is now a growing list of promising molecules and mechanisms capable of alleviating cone death in RP.

### Example 2: Methods

Animals. CD-1 (#022) and FVB (rd1) (#207) mice were purchased from Charles River Laboratories. Sighted FVB (#004828), rd10 (#004297), C3H (rd1) (#000659), sighted C3H (#003648), and CX3CR1GFP/+ (#005582) mice were purchased from The Jackson Laboratory. Rhodopsin null (Rho-/-) mice were obtained (29). FVB and CX3CR1GFP/+ mice were crossed for at least four generations to obtain rd1;CX3CRlGFP/+ animals. Mice were subsequently bred and maintained in a facility on a 12-hour alternating light and dark cycle. Both male and female mice were used in all experiments.

Histology. To prepare retinal cross-sections, enucleated eyes were dissected in phosphate-buffered saline (PBS) to remove the cornea, iris, lens, and ciliary body. The remaining eye cup was fixed in 4% paraformaldehyde for one hour at room temperature, cryoprotected in a sucrose gradient, and embedded in a 1:1 solution of 30% in PBS and optimal cutting temperature compound (Tissue-Tek) on dry ice. Frozen eye cups were cut on a Leica CM3050S cryostat (Leica Microsystems) into 20-30 µm sections. If applicable, sections were then blocked for one hour at room temperature in PBS containing 5% goat serum and 0.1% Triton X-100, stained with primary antibodies (Table 4) in blocking buffer overnight at 4oC, and incubated with the appropriate secondary antibody in PBS for two hours at room temperature. All sections were incubated for five minutes at room temperature with PBS containing 0.5 µg/mL of 4′,6-diamidino-2-phenylindole (DAPI) (Thermo Fisher Scientific) and mounted using Fluoromount-G (SouthernBiotech). To prepare retinal flat-mounts for quantifying GFP-positive cones, whole retinas were fixed in 4% paraformaldehyde for 30 minutes at room temperature. After PBS washes, retinas were relaxed with four radial incisions, flattened onto a microscope slide, and mounted with the ganglion cell layer facing up. Descriptions of additional histology procedures can be found in the Supplemental Materials and Methods.

Microglial depletion. Microglia were depleted using PLX5622 (a gift from Plexxikon, Berkeley, CA, USA), an orally available CSF1R inhibitor, formulated into AIN-76A rodent chow (Research Diets) at 1200 mg/kg and provided ad libitum.

Flow cytometry and cell sorting. All flow cytometry and cell sorting were performed on a BD FACSAria II and analyzed using FlowJo 10 (Tree Star). For retinal cells, freshly dissected retinas were dissociated as previously described (12) using cysteine-activated papain followed by gentle trituration with a micropipette. If applicable, harvested cells were then blocked for five minutes with 1:100 of rat anti-mouse CD16/32 (BD Pharmingen®) and incubated for 20 minutes on ice with the antibodies listed in Table 4. Prior to analysis, all samples were passed through a 40 µm filter and stained with 0.5 µg/mL of DAPI (Thermo Fisher Scientific®) in FACS buffer (PBS containing 2% fetal bovine serum and 2 mM ethylenediaminetetraacetic acid [EDTA]) to exclude non-viable cells. Descriptions of additional flow cytometry procedures can be found in the Supplemental Materials and Methods.

RT-PCR. mRNA was isolated from whole retinas or sorted microglia using an RNeasy Micro Kit (Qiagen®) as previously described (12), with the exception of mRNA from Rho-/- retinas, which was isolated from fixed tissues using the RecoverAll Total Nucleic Acid Isolation Kit for FFPE (Thermo Fisher Scientific®). One whole retina or 1000-2000 sorted microglia were collected per sample. cDNA was synthesized using the SuperScript III First-Strand Synthesis System (Invitrogen®) with oligo(dT) primers, followed by RT-PCR using the Power SYBR Green™ PCR Master Mix (Applied Biosystems®) on a CFX96 real-time PCR detection system (BioRad®). Reactions were performed in triplicate with expression normalized to the housekeeping gene Gapdh. Sequences for RT-PCR primers were designed using PrimerBank (42) and are listed in Table 5. For Rho-/- samples, primers targeting shorter amplicons (Gapdh-s and Tgfb1-s) were used to account for potential fragmentation of mRNA following fixation.

AAV vector design and production. The AAV-human red opsin-GFP-WPRE-bGH (AAV8-GFP) plasmid was a gift from Botond Roska (Friedrich Miescher Institute for Biomedical Research, Basel, Switzerland) (43). To generate plasmids for TGF-β isoforms, the GFP coding sequence from AAV8-GFP was replaced with the full-length mouse cDNA for TGF-β1 (NM_011577.2), TGF-β2 (NM_009367.4), or TGF-β3 (NM_009368.3) flanked by NotI and AgeI restriction sites. Recombinant AAV serotype 8 (AAV8) vectors were produced as previously described (11, 12, 44). Briefly, HEK293T cells were transfected using polyethylenimine with a mixture of the vector plasmid, adenovirus helper plasmid, and rep2/cap8 packaging plasmid. Supernatant was harvested 72 hours post-transfection, and viral particles were PEGylated overnight and precipitated by centrifugation. Viral particles were subsequently centrifuged through an iodixanol gradient to remove cellular debris, and the recovered vectors were washed three times with PBS and collected in a final volume of 100-200 µL. AAV vectors were semi-quantitatively titered by SYPRO Ruby (Molecular Probes®) staining for viral capsid proteins (VP1, VP2, and VP3) in comparison to a reference vector titered by RT-PCR.

Subretinal injections. All subretinal injections were performed on neonatal mice at P0-P1. After anesthetization of the mouse on ice, the palpebral fissure was carefully opened with a 30-gauge needle and the eye exposed. Using a glass needle controlled by a FemtoJet™ microinjector (Eppendorf®), ~0.25 µL of AAV vectors was then injected into the subretinal space. For each eye, 5 x 10⁸ vector genomes (vg) per eye of AAV8-GFP were administered. All other vectors were administered at 1 × 10⁹ vg per eye.

Image acquisition and analysis. Images of retinal cross-sections and GFP-positive cones in retinal flat-mounts were acquired using a Zeiss® LSM710 scanning confocal microscope (20x air objective or 40x oil objective) and Nikon® Ti inverted wide-field microscope (10x air objective), respectively. All image analysis was performed using ImageJ. Quantification of GFP-positive cones was performed as previously described (12) using a custom ImageJ module (available at https://sites.imagej.net/Seankuwang/). For each flat-mount, the user indicated the location of the optic nerve head and each of the four retinal leaflets. The module then automatically defined the region corresponding to the central retina and counted the number of GFP-positive objects within the region. This value was used to represent the number of GFP-positive cones in the central retina for each sample. Quantification of microglia in the ONL of retinal cross-sections was performed by dividing the number of microglia in the ONL across five random fields by the total number of microglia in those fields. Microglia were defined as residing in the ONL if 50% or more of the cell body was located in that layer. Descriptions of additional image analysis procedures can be found in EXAMPLE 3.

Light-dark discrimination. Innate light-avoidance behavior in mice was assessed as previously described (45) with minor modifications. A 28 cm (length) by 28 cm (width) by 21 cm (height) plastic chamber (Med Associates) was divided into two equally sized compartments: one dark and one brightly illuminated (~900 lux). Temperatures in the two compartments differed by less than 1° C. A small opening connected the two compartments, allowing subjects to freely travel throughout the chamber. At the beginning of each trial, a mouse was placed in the illuminated compartment and its activity recorded for ten minutes. If after one minute, the animal had not yet entered the dark compartment, it was gently directed there, removed from the chamber, and the trial restarted. The location and movement of each mouse were determined by infrared sensors and analyzed with Activity Monitor (Med Associates). Percentage of time spent in dark was calculated based on activity during the final nine minutes of each trial.

Optomotor assay. Visual acuity was measured by an observer (YX) blinded to the treatment assignment using the OptoMotry System (CerebralMechanics™) as previously described (11, 12, 46). Mice were placed inside a virtual-reality chamber with bright background luminance to saturate rods and presented with sine wave gratings of varying spatial frequencies. During each test, the observer assessed reflexive head-tracking movements of the animal in response to the sine wave grating, and for each eye, the highest spatial frequency at which the animal tracked the grating was determined to be the visual acuity. Left and right eyes were tested independently using clockwise and counterclockwise gratings, respectively, as only motion in the temporal-to-nasal direction is known to evoke the optomotor response in mice (4).

RNA sequencing. Transcriptional profiling of microglia (seven biological replicates per experimental condition) or non-microglia (four biological replicates total) was performed as previously described (12). One thousand microglia (CD11b+Ly6G/Ly6C-) or non-microglia cells (CD11b-) from each retina were sorted into 10 µL of Buffer TCL (Qiagen®) containing 1% beta-mercaptoethanol and immediately frozen on dry ice. Samples were subsequently sent to the Broad Institute Genomics Platform for cDNA library synthesis and sequencing using a modified Smart-Seq2 protocol (47) with an expected coverage of ~6 million reads per sample. Prior to analysis, reads were subjected to quality control measures and mapped to the GRCm38.p6 reference genome. Reads assigned to each gene were then quantified using featureCounts (48) and normalized and analyzed for differential expression using DESeq2 (49). All RNA-seq data generated in this work have been deposited in the Gene Expression Omnibus (GEO) repository (accession number GSE145601).

TGFBR½ inhibition. Pharmacological inhibition of the TGF-β type I and II receptors in vivo was performed using a combination of SB431542 (SelleckChem®) and LY364947 (SelleckChem) as previously described (36). Both compounds were dissolved in PBS containing 5% dimethyl sulfoxide (DMSO) and 30% polyethylene glycol 300 and dosed at 10 mg/kg daily via intraperitoneal injections.

Statistics. All group data are shown as mean ± SEM. Two-tailed Student’s t-tests were used to compare experimental groups, with the addition of a Bonferroni correction if three or more hypotheses were tested. Differences between groups were considered significant when the P-value was less than 0.05.

### References

1. Daiger SP, Sullivan LS, Bowne SJ. Genes and mutations causing retinitis pigmentosa. Clin. Genet. 2013;84(2):132-141.

2. Merin S, Auerbach E. Retinitis pigmentosa. Surv. Ophthalmol. 1976;20(5):303-346.

3. Hartong DT, Berson EL, Dryja TP. Retinitis pigmentosa. Lancet 2006;368(9549):1795-1809.

4. Narayan DS, Wood JPM, Chidlow G, Casson RJ. A review of the mechanisms of cone degeneration in retinitis pigmentosa. Acta Ophthalmol. 2016;94(8):748-754.

5. N. Sahni J et al. Therapeutic Challenges to Retinitis Pigmentosa: From Neuroprotection to Gene Therapy. Curr. Genomics 2011;12(4):276-284.

6. Huang XF. Current pharmacological concepts in the treatment of the retinitis pigmentosa. In: Advances in Experimental Medicine and Biology. 2018:439-445

7. Russell S et al. Efficacy and safety of voretigene neparvovec (AAV2-hRPE65v2) in patients with RPE65-mediated inherited retinal dystrophy: a randomised, controlled, open-label, phase 3 trial. Lancet 2017;390(10097):849-860.

8. Trapani I, Banfi S, Simonelli F, Surace EM, Auricchio A. Gene Therapy of Inherited Retinal Degenerations: Prospects and Challenges. Hum. Gene Ther. 2015;26(4):193-200.

9. Daiger SP, Bowne SJ, Sullivan LS. Perspective on genes and mutations causing retinitis pigmentosa. Arch. Ophthalmol. 2007;125(2):151-158.

10. Byrne LC et al. Viral-mediated RdCVF and RdCVFL expression protects cone and rod photoreceptors in retinal degeneration. J. Clin. Invest. 2015;125(1): 105-116.

11. Xiong W, Garfinkel AEM, Li Y, Benowitz LI, Cepko CL. NRF2 promotes neuronal survival in neurodegeneration and acute nerve damage. J. Clin. Invest. 2015;125(4):1433-1445.

12. Wang SK, Xue Y, Rana P, Hong CM, Cepko CL. Soluble CX3CL1 gene therapy improves cone survival and function in mouse models of retinitis pigmentosa. Proc. Natl. Acad. Sci. U. S. A. 2019;116(20):10140-10149.

13. Block ML, Zecca L, Hong J-S. Microglia-mediated neurotoxicity: uncovering the molecular mechanisms. Nat. Rev. Neurosci. 2007;8(1):57-69.

14. Subhramanyam CS, Wang C, Hu Q, Dheen ST. Microglia-mediated neuroinflammation in neurodegenerative diseases. Semin. Cell Dev. Biol. 2019;94:112-120.

15. Smith JA, Das A, Ray SK, Banik NL. Role of pro-inflammatory cytokines released from microglia in neurodegenerative diseases. Brain Res. Bull. 2012;87(1): 10-20.

16. Zhao L et al. Microglial phagocytosis of living photoreceptors contributes to inherited retinal degeneration. EMBO Mol. Med. 2015;7(9): 1179-1197.

17. Peng B et al. Suppression of Microglial Activation Is Neuroprotective in a Mouse Model of Human Retinitis Pigmentosa. J. Neurosci. 2014;34(24):8139-8150.

18. Kim W-K et al. TGF-β1 Represses Activation and Resultant Death of Microglia via Inhibition of Phosphatidylinositol 3-Kinase Activity. J. Immunol. 2004;172(11):7015-7023.

19. Taylor RA et al. TGF-β1 modulates microglial phenotype and promotes recovery after intracerebral hemorrhage. J. Clin. Invest. 2017;127(1):280-292.

20. Elmore MRP et al. Colony-stimulating factor 1 receptor signaling is necessary for microglia viability, unmasking a microglia progenitor cell in the adult brain. Neuron 2014;82(2):380-97.

21. Chang B et al. Retinal degeneration mutants in the mouse. Vision Res. 2002;42(4):517-525.

22. Bennett ML et al. New tools for studying microglia in the mouse and human CNS. Proc. Natl. Acad. Sci. U. S. A. 2016;113(12):E1738-E1746.

23. Liddelow SA et al. Neurotoxic reactive astrocytes are induced by activated microglia. Nature 2017;541(7638):481-487.

24. Chitnis T, Weiner HL. CNS inflammation and neurodegeneration. J. Clin. Invest. 2017;127(10):3577-3587.

25. Wrana JL, Attisano L, Wieser R, Ventura F, Massagué J. Mechanism of activation of the TGF-β receptor. Nature 1994;370(6488):341-347.

26. Zöller T et al. Silencing of TGFβ signalling in microglia results in impaired homeostasis. Nat. Commun. 2018;9(1):4011.

27. Ma W et al. Absence of TGFβ signaling in retinal microglia induces retinal degeneration and exacerbates choroidal neovascularization. Elife 2019;8. doi:10.7554/eLife.42049

28. Li Q, Timmers AM, Guy J, Pang J, Hauswirth WW. Cone-specific expression using a human red opsin promoter in recombinant AAV. Vision Res. 2008;48(3):332-338.

29. Lem J et al. Morphological, physiological, and biochemical changes in rhodopsin knockout mice. Proc. Natl. Acad. Sci. U. S. A. 1999;96(2):736-741.

30. Douglas RM et al. Independent visual threshold measurements in the two eyes of freely moving rats and mice using a virtual-reality optokinetic system. Vis. Neurosci. 2005;22(5):677-684.

31. Srinivasan Y, Lovicu FJ, Overbeek PA. Lens-specific expression of transforming growth factor β1 in transgenic mice causes anterior subcapsular cataracts. J. Clin. Invest. 1998;101(3):625-634.

32. Robertson J V., Golesic E, Gauldie J, West-Mays JA. Ocular gene transfer of active TGF-β induces changes in anterior segment morphology and elevated IOP in rats. Investig. Ophthalmol. Vis. Sci. 2010;51(1):308-318.

33. Dvashi Z, Goldberg M, Adir O, ShapiraM, Pollack A. TGF-β1 induced transdifferentiation of RPE cells is mediated by TAK1. PLoS One 2015;10(4):e0122229.

34. Yang S, Yao H, Li M, Li H, Wang F. Long non-coding RNA MALAT1 mediates transforming growth factor beta1-induced epithelial-mesenchymal transition of retinal pigment epithelial cells. PLoS One 2016;11(3):e0152687.

35. Georgiadis A et al. The tight junction associated signalling proteins ZO-1 and ZONAB regulate retinal pigment epithelium homeostasis in mice. PLoS One 2010;5(12):e15730.

36. Maddaluno L et al. EndMT contributes to the onset and progression of cerebral cavernous malformations. Nature 2013;498(7455):492-496.

37. Feng X et al. Microglia mediate postoperative hippocampal inflammation and cognitive decline in mice. JCI insight 2017;2(7):e91229.

38. O′Koren EG et al. Microglial Function Is Distinct in Different Anatomical Locations during Retinal Homeostasis and Degeneration. Immunity 2019;50(3):723-737.e7.

39. Zaiss A-K et al. Differential Activation of Innate Immune Responses by Adenovirus and Adeno-Associated Virus Vectors. J. Virol. 2002;76(9):4580-4590.

40. Tamiya S, Liu LH, Kaplan HJ. Epithelial-mesenchymal transition and proliferation of retinal pigment epithelial cells initiated upon loss of cell-cell contact. Investig. Ophthalmol. Vis. Sci. 2010;51(5):2755-2763.

41. Milam AH, Li ZY, Fariss RN. Histopathology of the human retina in retinitis pigmentosa. Prog. Retin. Eye Res. 1998;17(2):175-205.

42. Wang X, Spandidos A, Wang H, Seed B. PrimerBank: A PCR primer database for quantitative gene expression analysis, 2012 update. Nucleic Acids Res. 2012;40(D1):D1144-D1149.

43. Busskamp V et al. Genetic Reactivation of Cone Photoreceptors Restores Visual Responses in Retinitis Pigmentosa. Science 2010;329(5990):413-417.

44. Grieger JC, Choi VW, Samulski RJ. Production and characterization of adeno-associated viral vectors. Nat. Protoc. 2006;1(3):1412-1428.

45. Gaub BM et al. Restoration of visual function by expression of a light-gated mammalian ion channel in retinal ganglion cells or ON-bipolar cells. Proc. Natl. Acad. Sci. U. S. A. 2014;111(51):E5574-E5583.

46. Xiong W et al. AAV cis-regulatory sequences are correlated with ocular toxicity. Proc. Natl. Acad. Sci. U. S. A. 2019;116(12):5785-5794.

47. Picelli S et al. Smart-seq2 for sensitive full-length transcriptome profiling in single cells. Nat. Methods 2013;10(11):1096-1100.

48. Liao Y, Smyth GK, Shi W. FeatureCounts: An efficient general purpose program for assigning sequence reads to genomic features. Bioinformatics 2014;30(7):923-930.

49. Anders S, Huber W. Differential expression analysis for sequence count data. Genome Biol. 2010;11(10):R106.

The contents of each of the references provided herein are incorporated by reference in their entirety.

### Example 3: Supplemental Materials and Methods

Flow cytometry of non-retinal populations. All flow cytometry was performed on a BD FACSAria II and analyzed using FlowJo 10 (Tree Star®). For peripheral blood cells, 50 µL of tail vein blood from each mouse was collected in phosphate-buffered saline (PBS) containing 2 mM ethylenediaminetetraacetic acid (EDTA) and red blood cells lysed using BD Pharm Lysing Buffer according to manufacturer’s instructions. For peritoneal cells, 10 mL of FACS buffer (PBS containing 2% fetal bovine serum and 2 mM EDTA) was injected into the peritoneal cavity of each animal shortly after sacrifice. Following peritoneal massage, the buffer was collected and centrifuged at 400 x g for ten minutes to precipitate peritoneal cells. Harvested peritoneal cells were blocked for five minutes with 1:100 of rat anti-mouse CD16/32 (BD Pharmingen®) and incubated for 20 minutes on ice with the antibodies listed in Table 4. Prior to analysis, all samples were passed through a 40 µm filter and stained with 0.5 µg/mL of 4′,6-diamidino-2-phenylindole (DAPI) (Thermo Fisher Scientific®) in FACS buffer to exclude non-viable cells.

Ex vivo retinal culture. Freshly isolated retinas were relaxed with four radial incisions and placed on a 12 mm Millicell cell culture insert (Millipore®) resting on 2 mL of prewarmed culture media with the ganglion cell layer facing up. Culture media consisted of a 1:1 ratio of DMEM and F-12 supplemented with L-glutamine, B27, N2, and penicillin-streptomycin. Explants were maintained in humidified incubators at 37° C. and 5% CO2 for 48-72 hours, after which the media was assayed for TGF-β1, TGF-β2, or TGF-β3 protein using commercial ELISA kits (R&D Systems®). All ELISA reactions were performed in triplicate.

Immunostaining of flat-mounts. Freshly dissected retinas were fixed in 4% paraformaldehyde for 30 minutes at room temperature. After PBS washes, retinas were blocked for one hour in PBS containing 5% donkey serum and 0.3% Triton X-100 and stained with either anti-cone arrestin overnight or anti-BRN3A for two nights at 4° C. (see Table 4 for additional details). For retinal pigment epithelium (RPE) preparations, enucleated eyes were dissected to remove the cornea, iris, lens, ciliary body, retina, and connective tissue. The remaining RPE-choroid-sclera complex was fixed in 4% paraformaldehyde for one hour at room temperature, blocked in PBS containing 5% donkey serum and 0.3% Triton X-100 for one hour, and stained with anti-ZO-1 for two nights at 4° C. (see Table 4 for additional details). All samples were subsequently incubated with the appropriate secondary antibody in PBS for two hours at room temperature, relaxed with four radial incisions, and flat-mounted onto microscope slides. Images of cone arrestin immunostaining in retinal flat-mounts were acquired using a Nikon® Ti inverted widefield microscope (20x air obj ective). Images of BRN3A and ZO-1 immunostaining in flat-mounted retinas and RPE preparations, respectively, were acquired in the mid-periphery using a Zeiss® LSM710 scanning confocal microscope (20x air objective) and displayed as maximum intensity projections.

Cone arrestin quantification. Quantification of cone arrestin (CAR)-positive cones was performed similarly to that of GFP-positive cones using a custom ImageJ module (available at sites.imagej.net/Seankuwang). For each flat-mount, the user indicated the location of the optic nerve head and each of the four retinal leaflets. The module then automatically defined the region corresponding to the central retina and counted the number of CAR-positive objects within the region. This value was used to represent the number of CAR-positive cones in the central retina for each sample.

Cataract examination. Mice were examined for cataracts in vivo by a blinded observer using the Micron IV fundus imaging system (Phoenix Research Labs). Following anesthetization of animals with a mixture of ketamine/xylazine (100/10 mg/kg), pupils were dilated with a drop of 0.5% tropicamide and eyes hydrated with Gonak 2.5% hypromellose solution (Akorn). Images of isolated lenses in PBS were acquired using a Leica M165 FC dissecting microscope.

TABLE 4

List of antibodies

| Antibody | Vendor | Catalog # | Application | Dilution |
|---|---|---|---|---|
| PE-Cy5-conjugated anti-CD11b | BioLegend® | 101209 | FC | 1:200 |
| FITC-conjugated anti-F4/80 | BioLegend® | 123107 | FC | 1:200 |
| APC-Cy7-conjugated anti-Ly6C | BioLegend® | 128025 | FC | 1:200 |
| APC-Cy7-conjugated anti-Ly6G | BioLegend® | 127623 | FC | 1:200 |
| Rabbit anti-IBA1 | Thermo Fisher Scientific® | PA5-21274 | IHC (section) | 1:1000 |
| Rabbit anti-TGFBR2 | Abcam® | ab61213 | IHC (section) | 1:100 |
| Mouse anti-α-smooth muscle actin | Sigma-Aldrich® | A5228 | IHC (section) | 1:1000 |
| Goat anti-rabbit Alexa Fluor 594 | Jackson ImmunoResearch® | 111-585- 144 | IHC (section) | 1:1000 |
| Donkey anti-mouse Alexa Fluor 594 | Jackson ImmunoResearch® | 715-585- 150 | IHC (section and flat-mount) | 1:1000 |
| Rabbit anti-cone arrestin | EMD Millipore® | AB15282 | IHC (flat-mount) | 1:3000 |
| Mouse anti-BRN3A | Santa Cruz Biotechnology® | sc-8429 | IHC (flat-mount) | 1:100 |
| Rabbit anti-ZO-1 | Thermo Fisher Scientific® | 61-7300 | IHC (flat-mount) | 1:100 |
| Donkey anti-rabbit Alexa Fluor 594 | Jackson ImmunoResearch® | 711-585- 152 | IHC (flat-mount) | 1:1000 |

FC, flow cytometry; IHC, immunohistochemistry

TABLE 5

List of RT-PCR primers

| | | |
|---|---|---|
| Gene | 5′ | 3′ |
| Clqa | AAAGGCAATCCAGGCAATATCA (SEQ ID NO: 19) | TGGTTCTGGTATGGACTCTCC (SEQ ID NO: 20) |
| Gapdh | AGGTCGGTGTGAACGGATTTG (SEQ ID NO: 21) | TGTAGACCATGTAGTTGAGGTCA (SEQ ID NO: 22) |
| Gapdh-s | TGACCTCAACTACATGGTCTACA (SEQ ID NO: 23) | CTTCCCATTCTCGGCCTTG (SEQ ID NO: 24) |
| Gas6 | TGCTGGCTTCCGAGTCTTC (SEQ ID NO: 25) | CGGGGTCGTTCTCGAACAC (SEQ ID NO: 26) |
| Il1a | CGAAGACTACAGTTCTGCCATT (SEQ ID NO: 27) | GACGTTTCAGAGGTTCTCAGAG (SEQ ID NO: 28) |
| Il1b | GCAACTGTTCCTGAACTCAACT (SEQ ID NO: 29) | ATCTTTTGGGGTCCGTCAACT (SEQ ID NO: 30) |
| Il6 | TAGTCCTTCCTACCCCAATTTCC (SEQ ID NO: 31) | TTGGTCCTTAGCCACTCCTTC (SEQ ID NO: 32) |
| Spp1 | AGCAAGAAACTCTTCCAAGCAA (SEQ ID NO: 33) | GTGAGATTCGTCAGATTCATCCG (SEQ ID NO: 34) |
| Tgƒb1 | CTCCCGTGGCTTCTAGTGC (SEQ ID NO: 35) | GCCTTAGTTTGGACAGGATCTG (SEQ ID NO: 36) |
| Tgƒb1-s | GAGCCCGAAGCGGACTACTA (SEQ ID NO: 37) | TGGTTTTCTCATAGATGGCGTTG (SEQ ID NO: 38) |
| Tgfb2 | CTTCGACGTGACAGACGCT(SEQ ID NO: 39) | GCAGGGGCAGTGTAAACTTATT (SEQ ID NO: 40) |
| Tgfb3 | CCTGGCCCTGCTGAACTTG (SEQ ID NO: 41) | TTGATGTGGCCGAAGTCCAAC (SEQ ID NO: 42) |
| Tmem119 | CCTACTCTGTGTCACTCCCG (SEQ ID NO: 43) | CACGTACTGCCGGAAGAAATC (SEQ ID NO: 44) |
| Tnƒ | CCCTCACACTCAGATCATCTTCT (SEQ ID NO: 45) | GCTACGACGTGGGCTACAG (SEQ ID NO: 46) |

### Supplemental References

1. Rothe G et al. Peripheral blood mononuclear phagocyte subpopulations as cellular markers in hypercholesterolemia. Arterioscler. Thromb. Vasc. Biol. 1996;16(12):1437-1447.

2. Georgiadis A et al. The tight junction associated signalling proteins ZO-1 and ZONAB regulate retinal pigment epithelium homeostasis in mice. PLoS One 2010;5(12):e15730.

The contents of each of the references provided herein are incorporated by reference in their entirety.

1. An engineered vector comprising:
a retina-specific promoter operably linked to a nucleic acid sequence encoding a transforming growth factor beta (TGF-β) polypeptide.

2. The engineered vector of claim 1, wherein the engineered vector is selected from the group consisting of: an adeno-associated virus (AAV) vector; an adenovirus vector; and a lentiviral vector.

3. The engineered vector of claim 2, wherein the AAV vector is selected from the group consisting of: serotype AAV8; AAV2; AAV5; and AAV2/8.

4. (canceled)

5. The engineered vector of claim 1, which comprises a Woodchuck Hepatitis Virus (WHV) Posttranscriptional Regulatory Element (WPRE).

6. The engineered vector of claim 1, wherein the TGF-β polypeptide is a TGF-β1 or TGF-β3 polypeptide.

7. The engineered vector of claim 1, wherein the retina-specific promoter is a red opsin promoter.

8. A pharmaceutical composition for the treatment of an ocular disease, the composition comprising:
(a) the engineered vector of claim 1; and
(b) a pharmaceutically acceptable carrier.

9. The pharmaceutical composition of claim 8, wherein the pharmaceutical composition is formulated for delivery to the eye.

10-12. (canceled)

13. A method of treating an ocular disease in a subject, the method comprising:
administering to the subject the pharmaceutical composition of claim 8.

14. (canceled)

15. The method of claim 13, wherein the ocular disease is selected from the group consisting of: retinitis pigmentosa; glaucoma; age-related macular degeneration; retinitis; sclerotic retinal maculodystrophy; diabetic retinopathy; proliferative retinopathy; toxic retinopathy; and retinopathy of prematurity.

16-18. (canceled)

19. A method of promoting cone survival in the retina of a subject, the method comprising:
intraocularly administering to the subject an effective amount of a composition comprising a vector comprising a nucleic acid construct comprising a retina-specific promoter operably linked to nucleic acid sequence encoding a transforming growth factor beta (TGF-β) polypeptide.

20. The method of claim 19, wherein the vector is an adenovirus vector, an AAV vector or a lentiviral vector.

21. The method of claim 19, wherein the TGF-β polypeptide is a TGF-β1 or TGF-β3 polypeptide.

22-24. (canceled)

25. The method of claim 19, wherein the subject has or is suspected of having a neurodegenerative ocular disease.

26-28. (canceled)

29. A method of promoting neuronal cell survival, the method comprising: delivering a TGF-β polypeptide to a microglial cell.

30. The method of claim 29, wherein the TGF-β polypeptide is a TGF-β1 or TGF-β3 polypeptide.

31. The method of claim 29, wherein the delivering comprises administering a vector encoding the TGF-β polypeptide to a neuronal cell associated with the microglial cell.

32. The method of claim 31, wherein the vector is selected from the group consisting of: an adeno-associated virus (AAV) vector; an adenovirus vector; and a lentiviral vector.

33. (canceled)

34. The method of claim 31, wherein the vector comprises a promoter active in a neuronal cell, operatively linked to nucleic acid sequence encoding the TGF-β polypeptide.

35-36. (canceled)

37. The method of claim 34, wherein the promoter is a red opsin promoter.

38-50. (canceled)
